(19) United States
(12) Patent Application Publication
(10) Pub. No.: US 2023/0280334 A1
(43) Pub. Date: Sep. 7, 2023

(54) CDC20 VARIANTS RESISTANT TO ANTI-MITOTIC DRUGS AND RELATED METHODS AND COMPOSITIONS

(71) Applicant: Whitehead Institute for Biomedical Research, Cambridge, MA (US)

(72) Inventors: Mary Jane Tsang Mui Ching, Melbourne (AU); Iain Cheeseman, Brookline, MA (US)

(21) Appl. No.: 17/878,774

(22) Filed: Aug. 1, 2022

Related U.S. Application Data
(60) Provisional application No. 63/228,001, filed on Jul. 30, 2021.

(51) Int. Cl. G01N 33/50; C12N 15/113

(52) U.S. Cl. CPC G01N 33/5011; C12N 15/113; G01N 2500/10

(57) ABSTRACT

Disclosed are methods of screening for agents to treat cancers resistant to anti-mitotic therapy, methods of detecting cancers resistant to anti-mitotic therapy, and methods of treating cancers resistant to anti-mitotic therapy.

### CROSS-REFERENCE TO RELATED APPLICATION(S)

This application claims the benefit of U.S. Provisional Application No.: 63/228,001, filed Jul. 30, 2021. The entire teachings of the above application are incorporated herein by reference.

### GOVERNMENT SUPPORT

This invention was made with government support under GM088313 and 5R35GM126930 awarded by the National Institutes of Health. The government has certain rights in the invention.

### STATEMENT REGARDING SEQUENCE LISTING

The Sequence Listing associated with this application is provided in .xml format in lieu of a paper copy and is hereby incorporated by reference into the specification. The name of the .xml file containing the Sequence Listing is WIBR-178-101.xml. The xml file is 73,780 bytes, was created on Mar. 1, 2023, and is being submitted electronically via Patent Center.

### BACKGROUND OF THE INVENTION

Drugs that block cell division are among the most effective therapeutics used during cancer chemotherapy. These chemotherapeutic agents, such as Taxol, elicit a prolonged mitotic arrest that ultimately leads to cell death. Unfortunately, cells can escape this arrest by “slipping” out of mitosis into interphase. This mitotic slippage behavior varies dramatically between different cancer cell lines and tumors, and limits the efficacy of current anti-mitotic drugs. To maximize the power of these cancer treatments, it is critical to understand the molecular mechanisms responsible for these differences in mitotic slippage behavior. The Spindle Assembly Checkpoint (SAC) is the key regulatory pathway that governs this prolonged mitotic arrest and many cancer cells display compromised checkpoint activity. However, mutations in checkpoint genes are not commonly found in human tumor cells, suggesting that SAC impairment in cancers instead results from alterations at the transcriptional or translational level. Despite extensive work and an increasing understanding of the spindle assembly checkpoint, the factors that promote mitotic slippage in normal and cancer cells remain mysterious.

### SUMMARY OF THE INVENTION

Some aspects of the present disclosure are directed to a method of screening for a candidate anti-cancer agent, comprising (a) providing a cell expressing a Cdc20 variant and resistant to an anti-mitotic drug; (b) contacting the cell with an anti-mitotic drug and a test agent; (c) determining if the test agent reduces mitotic slippage as compared to a control; and (d) identifying the test agent as a candidate anti-cancer agent if the test agent reduces mitotic slippage as compared to the control. In some embodiments, the cell is a cancer cell. In some embodiments, the Cdc20 variant comprises an N-terminal truncation.

Some aspects of the present disclosure are directed to a method of determining if a subject with cancer is a candidate for anti-mitotic drug therapy, comprising (a) measuring the expression level of one or more Cdc20 variants in the cancer, and (b) determining that the subject is a candidate for anti-mitotic drug therapy if the expression level is below a threshold. In some embodiments, the method further comprises administering the anti-mitotic drug therapy to the subject determined to be a candidate for anti-mitotic drug therapy.

Some aspects of the present disclosure are directed to a method of determining if a subject with cancer is a candidate for anti-mitotic drug therapy, comprising (a) measuring the expression level of one or more Cdc20 variants and the expression level of Cdc20 wild-type in the cancer, and (b) determining that the subject is a candidate for anti-mitotic drug therapy if the ratio of the expression level of the one or more Cdc20 variants to Cdc20 wild-type is below a threshold. In some embodiments, the method further comprises administering the anti-mitotic drug therapy to the subject determined to be a candidate for anti-mitotic drug therapy.

Some aspects of the present disclosure are directed to a method of treating a subject in need thereof with and anti-mitotic drug therapy, comprising (a) identifying a subject having an expression level of one or more Cdc20 variants resistant to anti-mitotic drug therapy below a threshold, and (b) administering the anti-mitotic therapy to the subject.

Some aspects of the present disclosure are directed to a method of treating a subject in need thereof with and anti-mitotic drug therapy, comprising (a) identifying a subject having a ratio of an expression level of one or more Cdc20 variants resistant to anti-mitotic drug therapy to an expression level of Cdc20 wild-type below a threshold, and (b) administering the anti-mitotic therapy to the subject.

Some aspects of the present disclosure are directed to a method of inhibiting a cancer cell expressing a Cdc20 variant and resistant to an anti-mitotic drug comprising contacting the cancer cell with an agent that reduces the expression or activity of the Cdc20 variant and, optionally, the anti-mitotic drug. In some embodiments, the agent inhibits the binding of the Cdc20 variant with APC/C. In some embodiments, the agent inhibits the expression of the Cdc20 variant. In some embodiments, the agent increases the expression or activity of Cdc20 wild-type or a Cdc20 variant not resistant to the anti-mitotic drug. In some embodiments, the agent is the Cdc20 wild-type or the Cdc20 variant not resistant to the anti-mitotic drug, or a nucleotide sequence coding for the same. In some embodiments, the agent comprises residues 1-42 of SEQ ID NO: 2, or a functional fragment thereof.

Some aspects of the present disclosure are directed to a method of inhibiting a cancer cell expressing a Cdc20 variant and resistant to an anti-mitotic drug comprising contacting the cancer cell with an endonuclease and modifying the genome of the cancer cell, wherein the modification reduces or eliminates the expression of a Cdc20 variant resistant to an anti-mitotic drug or wherein the modification increases the expression of wild-type Cdc20.

In some embodiments, the endonuclease is a Cas9 nuclease and wherein the cancer cell is further contacted with one or more gRNA.

In some embodiments, the modification eliminates one or more Cdc20 alternate translation start sites (i.e., alternate start codons). In some embodiments, the alternate translation start site is located at positions 127-129 of SEQ ID NO: 1. In some embodiments, the alternate translation start site is located at positions 262-264 of SEQ ID NO: 1.

In some embodiments, the modification is at the translation start site (e.g., start codon) for wild-type Cdc20 and increases expression of wild-type Cdc20 (e.g., positions 127-129 or positions 262-264 of SEQ ID NO: 1). In some embodiments, the modification is changing the translation start site to the consensus Kozak sequence.

In some embodiments, the modified cell is contacted with the anti-mitotic drug.

Some aspects of the present disclosure are directed to a composition comprising a Cas9 nuclease or a nucleotide sequence encoding a Cas9 nuclease and one or more gRNA or a nucleotide sequence encoding one or more gRNA targeting a Cdc20 alternate translation start site, wherein the composition is capable of eliminating the alternate translation start site. In some embodiments, the composition comprises one or more viruses capable of transducing a nucleotide sequence encoding a Cas9 nuclease and one or more gRNA. In some embodiments, the composition comprises a liposome, a lipid-based particle, a nanoparticle, a microparticle, a polymeric particle, or other delivery vehicle delivering a Cas9 nuclease or a nucleotide sequence encoding a Cas9 nuclease and one or more gRNA or a nucleotide sequence encoding one or more gRNA targeting a Cdc20 alternate translation start site. In some embodiments, the composition is capable of introducing an insertion, deletion, or substitution into the alternate translation start site.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies - A Laboratory Manual, Cold Spring Harbor Laboratory Press, Cold Spring Harbor, 1988; Freshney, R.I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman’s The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V.A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1F show that full-length Cdc20 protein is not essential in human cells due to the presence of alternative Cdc20 isoforms. (FIG. 1A) Illustration highlighting the opposing roles of Cdc20 as the key target of the SAC and as an essential APC/C cofactor required for mitotic progression. Regulating the roles of Cdc20 in SAC signaling and mitotic exit may alter SAC efficacy and the extent of mitotic slippage (indicated in red). (FIG. 1B) Top, diagram of the Cdc20 open reading frame indicating the targeting of sgRNAs and the presence of critical motifs. Bottom, representative images of DNA staining (Hoechst) to show the mitotic arrest behavior of HeLa cells following treatment with Cdc20 siRNAs or using an inducible CRISPR/Cas9 gene targeting strategy with sgRNAs recognizing different regions within CDC20 gene. Numbers indicate the average percent mitotic cells for two experimental replicates. n > 800 cells per condition per replicate. Scale bar, 40 µm. (FIG. 1C) Western blot showing HeLa, ΔM1, or M1-stop cells treated with control or Cdc20 siRNAs. Endogenous Cdc20 protein was detected using antibodies recognizing the C-terminus of human Cdc20 (aa 450-499). β-actin was used as loading control. (FIG. 1D) Growth curves of control HeLa compared to the ΔM1 and M1-stop mutant cell lines. (FIG. 1E) Graph showing mitotic duration for control HeLa compared to the ΔM1 and M1-stop mutant cell lines. Each point represents a single cell. The bar represents the mean mitotic duration ± standard deviation across two experimental replicates. Statistics from Student’s two-sample t-Test with two-tailed distribution (**** = p < 0.0001, NS = not significant) (FIG. 1F) Representative images of DNA staining (Hoechst) to indicate the mitotic arrest behavior of HeLa, ΔM1, and M1-stop cells treated with control or Cdc20 siRNAs. Numbers indicate the percent mitotic cells. n > 175 cells per condition. Scale bar, 20 µm.

FIGS. 2A-2F show Cdc20 isoforms are produced by alternative translation initiation at downstream in-frame start codons. (FIG. 2A) Protein alignment of the N-terminal region of human Cdc20 compared to other mammals and tetrapod species with conserved motifs indicated. Conserved amino acids are indicated with an asterisk. Methionine residues are highlighted in red. Conserved Met1, Met43, and Met88 are bolded. Arrow indicates the location of indel mutations in the CDC20_M1-fs-M43 cell line. (FIG. 2B) Cdc20 tryptic peptides with N-terminal acetylation indicative of translation initiation were identified following immunoprecipitation-mass spectrometry of mitotically-enriched samples using an endogenous C-terminal GFP tag. The identified peptide sequence, number of peptide-spectrum matches (#PSMs), and the cross-correlation (Xcorr) value from the SEQUEST search are indicated. (FIG. 2C) Cdc20 peptides as in (B), except using the endopeptidase LysC and isolated using a C-terminal GFP tag in a cell line lacking the full-length Cdc20 protein (CDC20_M1-fs-M43). (FIG. 2D) Western blot showing the presence of translation initiation at the Met1, Met43, and/or Met88 start codons. Wild-type and the indicated Cdc20 mutants were expressed ectopically in mitotically-enriched M1-stop cells depleted of endogenous Cdc20 protein using RNAi. The translation products were detected by Western blotting with antibodies recognizing the human Cdc20 C-terminus (aa 450-499) (C-term Ab), the human Cdc20 N-terminus (aa 1-175) whose epitope likely lies upstream of amino acid 88 (N-term Ab), or the acetylated M88-terminus (Ac-M88 Ab). β-actin was used as loading control. (FIG. 2E) Representative live-cell fluorescence microscopy images of untreated or nocodazole-treated HeLa cells expressing the indicated N-terminal mEGFP-Cdc20 fusions with 5 ng/ml doxycycline. Images show maximum intensity projections of deconvolved Z-stacks of selected mitotic cells. Images were scaled individually to highlight kinetochore localization. Scale bar, 5 µm. (FIG. 2F) Top, Schematic of FACS analysis of cells constitutively expressing the indicated constructs and the sgExon3 guide RNA to determine the fraction of mitotic cells. Endogenous Cdc20 protein was depleted with Cas9 induction and the percent of GFP-positive cells in mitosis was quantified. High levels of histone H3 phosphorylated at serine residue 10 (pS10) were used as a marker of mitosis. Bottom, numbers indicate the average percent of GFP-positive cells in mitosis for the indicated construct from two experimental replicates. n > 550 GFP+ cells per construct per replicate.

FIGS. 3A-3F show truncated Cdc20 isoforms are inefficient targets of the SAC and promote mitotic slippage. (FIG. 3A) Cumulative frequency distribution for the fraction of cells in mitosis at the indicated time after entry into mitosis (mitotic arrest duration) for HeLa, ΔM1, and M1-stop cells treated with 10 µM STLC. n > 30 cells for each cell line. (FIG. 3B) Representative images of DNA staining (Hoechst) to show the mitotic arrest behavior of HeLa or M1-stop cells treated for 8 h with a range of anti-mitotic drugs that activate the SAC. Indicated is the average percent mitotic cells of two experimental replicates. n > 600 cells per condition per replicate. Scale bar, 40 µm. (FIG. 3C) Cumulative frequency distribution showing the fraction of mitotic cells over time post-mitotic entry for HeLa, ΔM1, and M1-stop cells treated with 10 µM STLC alone or in combination with the APC/C-inhibitor proTAME (12 µM). n > 80 cells per cell line per condition across two experimental replicates. (FIG. 3D) Cumulative frequency distribution for the fraction of cells in mitosis in the presence of 10 µM STLC for HeLa, ΔM1, and M1-stop cells expressing endogenous Cdc20 protein or upon Cdc20 replacement with ectopic wild-type Cdc20 cDNA. n ≥ 80 cells per cell line per condition across two experimental replicates. (FIG. 3E) Representative immunofluorescence images of Mad2 localization to kinetochores immuno-stained with anti-centromere antibodies (ACA). Images are maximum intensity projections of deconvolved Z-stacks of selected mitotic cells from control HeLa or mutant ΔM1 or M1-stop cell lines treated with nocodazole. Images were scaled individually to highlight kinetochore localization. Scale bar, 5 µm. (FIG. 3F) Cumulative frequency distribution of the fraction of mitotic cells over time post-mitotic entry for HeLa, ΔM1, and M1-stop cells treated with 10 µM STLC alone or in combination with the Mps1-inhibitor AZ3146 (4 µM). n > 80 cells per cell line per condition across two experimental replicates.

FIGS. 4A-4F show the relative levels of Cdc20 translational isoforms influence mitotic arrest duration. (FIG. 4A) Mitotic arrest duration of individual HeLa cells treated with 10 µM STLC and 50 ng/µl doxycycline to induce expression of the indicated doxycycline-inducible CDC20 constructs. Cells entering mitosis in the first 325 min of time lapse experiments were included in analyses. Open red circles indicate cells that exit mitosis. Closed black circles indicate cells that remained arrested in mitosis till the end of the time lapse. Bars correspond to median. n > 150 cells per cell line across two experimental replicates. (FIG. 4B) Mitotic arrest duration in the presence of 10 µM STLC for HeLa, ΔM1, or M1-stop cells expressing the wild-type CDC20 cDNA. n > 70 cells per cell line across two experimental replicates. (FIG. 4C) Schematic illustrating the approach to isolate clonal cell lines from the polyclonal M1-stop mutant expressing the wild-type CDC20 construct. Multiple clones were analyzed to assess the correlation between the mitotic arrest behavior of a given clone and the expression level of the integrated doxycycline-inducible CDC20 construct (see text for details). (FIG. 4D) Western blot showing representative clones of M1-stop mutant with low, medium, or high expression of the doxycycline-inducible wild-type CDC20 construct without induction or induced with 20 ng/µl doxycycline. Cdc20 was detected using antibodies recognizing the C-terminus of human Cdc20 (aa 450-499). β-actin was used as loading control. (FIG. 4E) Mitotic arrest duration in the presence of 10 µM STLC for representative clones of M1-stop mutant with low, medium, or high expression of the doxycycline-inducible wild-type CDC20 construct treated with 20 ng/ul doxycycline. n > 90 cells per cell line across two experimental replicates. (FIG. 4F) Mitotic arrest duration in the presence of 10 µM STLC and 20 ng/ul doxycycline for representative clones of M1-stop mutant with low, medium, or high expression of the doxycycline-inducible wild-type CDC20 construct. Cells were treated with Cdc20 siRNAs to deplete endogenous truncated Cdc20 isoforms. Low Cdc20 expression from the inducible CDC20 construct fails to support mitotic progression even before addition of STLC. n > 90 cells per cell line across two experimental replicates.

FIGS. 5A-5E show translation initiation at alternative out-of-frame start codons modulates Cdc20 isoform expression levels. (FIG. 5A) Analysis of human CDC20 nucleic acid sequence reveals two alternative out-of-frame start codons between Met1 and Met43. The amino acid sequence of the predicted alternative open reading frame (altORF) is indicated in cyan, with the methionines bolded. Mutations to alter the translation-initiation context of Met1 to either the consensus Kozak sequence (consKozak) or an antiKozak sequence (antiKozak) are underlined and highlighted in magenta. Targeted silent mutations to disrupt the out-of-frame start codons (altATGmutx2) or introduce an additional out-of-frame start codon before the Met43 start site (addATG) are similarly indicated. (FIG. 5B) Western blot showing CDC20 constructs with mutations to alter the translation-initiation context of Met1 to either the consensus Kozak sequence (consKozak) or an antiKozak sequence (antiKozak). Constructs were expressed in mitotically-enriched M1-stop cells depleted of endogenous Cdc20 protein. The translation products were detected using antibodies recognizing the human Cdc20 C-terminus (aa 450-499). β-actin was used as loading control. (FIG. 5C) Protein alignment of human Cdc20 altORF to other mammalian species. Conserved amino acids are indicated with an asterisk. Colors indicate related residues (hydrophobic, positively charged, negatively charged). The conserved stop site is present downstream of the Met88 start codon in each case. (FIG. 5D) Similar Western blot as in (B) except showing CDC20 constructs with targeted silent mutations to disrupt the out-of-frame start codons (altATGmutx2) or introduce an additional out-of-frame start codon before the Met43 start site (addATG). (FIG. 5E) Mitotic arrest duration in the presence of 10 µM STLC for individual HeLa cells in which the endogenous Cdc20 protein is replaced with either wild-type siRNA-resistant CDC20 cDNA or the altATGmutx2 mutant construct. Cells entering mitosis in the first 450 min of time lapse experiments were included in analyses. Open red circles indicate cells that exit mitosis. Closed black circles indicate cells that remained arrested in mitosis till the end of the time lapse. Bars correspond to median. Indicated are the median mitotic duration times across two experimental replicates and statistics from Mann-Whitney Test (**** = p < 0.0001).

FIGS. 6A-6D show downstream in-frame Cdc20 translation initiation influences the mitotic slippage behavior of cancer cell lines. (FIG. 6A) Mitotic arrest duration of individual HeLa, U2OS, or A549 cells treated with 10 µM STLC alone or with a combination of STLC and siRNA treatment (either control siRNAs or Cdc20 siRNAs). Cells entering mitosis in the first 600 min (HeLa) or 700 min (U2OS/A549) of time lapse experiments were included in analyses. Open red circles indicate cells that exit mitosis. Closed black circles indicate cells that remained arrested in mitosis till the end of the time lapse. Blue circles indicate cells that die in mitosis. Bars correspond to median. Indicated are the median mitotic duration times across two experimental replicates. (FIG. 6B) Western blot of mitotically-enriched U2OS cells expressing endogenous Cdc20 protein or upon Cdc20 replacement with either wild-type CDC20 cDNA or a CDC20 M43L M88L mutant construct. Cells were enriched in mitosis with 10 µM STLC for 18 hrs. Cdc20 protein was detected using antibodies recognizing the human Cdc20 C-terminus (aa 450-499). β-actin was used as loading control. (FIG. 6C) Western blot similar to (B) for A549 cells except that ectopic constructs were induced with 50 ng/ml doxycycline. (FIG. 6D) Top, schematic illustrating the Cdc20 replacement strategy with siRNA-resistant 5′ UTR-CDC20 cDNA constructs from a doxycycline-inducible promoter combined with depletion of endogenous Cdc20 protein by siRNA treatment. Cells were treated with 10 ng/ml (HeLa/U2OS) or 50 ng/ml (A549) doxycycline. Bottom, mitotic arrest duration in the presence of 10 µM STLC for individual HeLa, U2OS, or A549 cells with Cdc20 replacement with either wild-type CDC20 cDNA or a CDC20 M43L M88L mutant construct. Cells entering mitosis in the first 450 min (HeLa) or 600 min (U2OS/A549) of time lapse experiments were included in analyses. Open red circles indicate cells that exit mitosis. Closed black circles indicate cells that remained arrested in mitosis till the end of the time lapse. Bars correspond to median. Indicated are the median mitotic duration times across two experimental replicates and statistics from Mann-Whitney Test (**** = p < 0.0001).

FIGS. 7A-7F show relative levels of SAC-resistant Cdc20 translational isoforms promote mitotic slippage and increased cell viability even in the presence of mitotic perturbations. (FIG. 7A) Survey of tumors and cancer cell lines using public databases reveals multiple distinct genetic mutations within CDC20 that are predicted to selectively deplete the full-length Cdc20 protein or impact expression of the M43 isoform. Indicated are the genetic change, the sample ID or cell line containing the mutation, and the cancer type. (FIG. 7B-FIG. 7C) Sensitivity of HeLa or M1-stop cells to increasing concentrations of STLC (FIG. 7B) or Taxol (FIG. 7C). Cell viability was determined by MTT assay in triplicate following 72h drug treatment. Error bars indicate SEM of three (STLC) or four (Taxol) experimental replicates. Statistics from Student’s two-sample t-Test with two-tailed distribution comparing HeLa and M1-stop cell viabilities per drug concentration (* = p < 0.05, ** = p < 0.01). (FIG. 7D) An active spindle assembly checkpoint relies on the sequestration of Cdc20 protein within the MCC and the formation of APC/C-MCC-bound complexes to inhibit APC/C function and maintain the mitotic arrest. (FIG. 7E) When the Cdc20 (43-499) isoform is the only Cdc20 isoform present in cells, MCC formation is likely disrupted, thus resulting in a SAC defect and premature mitotic exit. (FIG. 7F) Cells express multiple Cdc20 isoforms, including the full-length Cdc20 protein, which is efficiently incorporated in the MCC when the SAC is active. APC/C complexes containing the Cdc20 (43-499) isoform could still be subject to SAC-mediated inhibition when bound to the MCC. However, the efficiency of APC/C-MCC association and APC/C inhibition may vary depending on which Cdc20 isoforms are present within the APC/C and/or APC/C-MCC complexes, thus allowing cells to escape from SAC inhibition and exit mitotic arrest. Therefore, competition between Cdc20 isoforms may determine SAC effectiveness and mitotic arrest duration.

FIGS. 8A-8E show human cells express alternative isoforms of Cdc20. (FIG. 8A) Western blot showing hTERT RPE-1 cells treated with control or Cdc20 siRNAs probed using using antibodies recognizing the C-terminus of human Cdc20 (aa 450-499). GAPDH was used as loading control. (FIG. 8B) Western blot showing mitotic HeLa cells collected by shake-off after overnight treatment with 330 nM nocodazole. Lysates alone, with buffer only, or with buffer and lambda phosphatase treatment were probed using antibodies recognizing the C-terminus of human Cdc20 (aa 450-499). GAPDH was used as loading control. (FIG. 8C) Schematic illustrating cell synchronization approach using a single thymidine arrest in S phase and subsequent release into the cell cycle. Addition of 330 nM nocodazole leads to a prolonged mitotic arrest. Lysates of HeLa cells harvested at the indicated time points after single thymidine release were separated by SDS-PAGE and endogenous Cdc20 protein was detected using antibodies recognizing the C-terminus of human Cdc20 (aa 450-499). β-actin was used as loading control. (FIG. 8D) Sequence information for the homozygous ΔM1 mutant cell line lacking the canonical M1 ATG start codon. The DNA sequence of the genomic locus was determined by next-generation sequencing. (FIG. 8E) Sequence information for the M1-stop mutant cell line showing insertions of 53 nt and 105 nt respectively after the L14 residue. Underlined are premature stop codons that are in-frame with the M1 ATG start codon for both CDC20 alleles. The DNA sequence of the genomic locus was determine by next-generation sequencing.

FIGS. 9A-9D show that the absence of full-length Cdc20 impairs SAC function and results in increased mitotic slippage in the presence of anti-mitotic drugs. (FIG. 9A) Cumulative frequency distribution for the fraction of cells in mitosis at the indicated time after entry into mitosis (mitotic arrest duration) for HeLa, ΔM1, and M1-stop cells treated with 10 µM STLC and 100 nM of either control siRNAs or Cdh1 siRNAs. (FIG. 9B) Mitotic duration of individual HeLa cells expressing doxycycline-inducible Cas9 and sgRNAs recognizing different regions within the CDC20 gene. Unperturbed mitotic progression or mitotic arrest behavior were monitored upon treatment with DMSO or 10 µM STLC, respectively. Cells entering mitosis in the first 350-400 min of time lapse experiments were included in the analyses. Open red circles indicate cells that exit mitosis. Closed black circles indicate cells that remained arrested in mitosis till the end of the time lapse. Bars correspond to the median. n ≥ 65 cells per cell line per condition across two experimental replicates. (FIG. 9C) Representative immunofluorescence images of Bub1 localization to kinetochores marked with anti-centromere antibodies (ACA). Shown are maximum intensity projections of deconvolved Z-stacks of selected mitotic cells from control HeLa or mutant ΔM1 or M1-stop cell lines treated with nocodazole. Images were scaled individually to highlight kinetochore localization. Scale bar, 5 µm. (FIG. 9D) Cumulative frequency distribution showing the fraction of mitotic cells over time post-mitotic entry for HeLa, ΔM1, and M1-stop cells treated with 10 µM STLC and either control siRNAs or Mad2 siRNAs.

FIG. 10 shows that the Cdc20 (43-499) isoform promotes mitotic slippage when over-expressed in control HeLa cells. Western blot showing HeLa cells treated with 50 ng/ml doxycycline to express the indicated doxycycline-inducible CDC20 constructs. Cdc20 protein was detected using antibodies recognizing the C-terminus of human Cdc20 (aa 450-499). β-actin was used as loading control.

FIGS. 11A-11C show translation initiation at alternative out-of-frame start codons in HeLa cells. (FIG. 11A) Schematic illustrating the strategy to assess whether translation initiation occurs at the alternative out-of-frame start codons. Cdc20 protein indicated in black; altORF peptide indicated in cyan. See text for details. (FIG. 11B) Analysis of wild-type human CDC20 nucleic acid sequence reveals two alternative out-of-frame start codons between Met1 and Met43. The amino acid sequence of the predicted alternative open reading frame (altORF) is indicated below the nucleic acid sequence, with the methionines bolded. Sequence information is shown for representative clones with indel mutations where at least one CDC20 allele results in a frame shift that connects the altORF peptide with amino acid sequences encoding downstream regions of Cdc20. The DNA sequence of genomic locus was determined by next-generation sequencing. Insertions are highlighted in red. When present, the amino acid sequence of the resulting altATG-Cdc20 peptide produced is shown. (FIG. 11C) Western blot showing mitotically-enriched control HeLa, M1-stop mutant, and representative clones with indel mutations where at least one CDC20 allele resulted in a frame shift that connects the altORF peptide with amino acid sequences encoding downstream regions of Cdc20. Endogenous Cdc20 protein was detected using antibodies recognizing the C-terminus of human Cdc20 (aa 450-499). β-actin was used as loading control.

FIGS. 12A-12B show increased cell viability of M1-stop mutant treated with anti-mitotic drugs. (FIG. 12A-FIG. 12B) Sensitivity of HeLa or M1-stop cells to increasing concentrations of Nocodazole (FIG. 12A) or the CENP-E inhibitor GSK923295 (FIG. 12B). Cell viability was determined by MTT assay in triplicate following 72 h drug treatment. Error bars indicate SEM of three (Nocodazole) or four (GSK923295) experimental replicates. Statistics from Student’s two-sample t-Test with two-tailed distribution comparing HeLa and M1-stop cell viabilities per drug concentration (* = p < 0.05, ** = p < 0.01).

FIGS. 13A-13C illustrates a strategy using pooled CRISPR/Cas9-based screening to identify genes that become essential in the absence of the SAC. (FIG. 13A) Control cells or SAC-defective cells are transduced with a genome-wide single-guide RNA (sgRNA) lentiviral library. Cells are propagated to allow gene inactivation via Cas9-mediated genomic cleavage. Relative abundances of sgRNAs between the initial and final cell populations are measured by deep sequencing. (FIG. 13B) The CRISPR gene score (CS) will be defined as the average log2 fold-change in the abundance of all sgRNAs targeting a given gene after 14 population doublings. Genes that are essential within a given cell line will result in a negative average CS. (FIG. 13C) Comparison of the CRISPR scores of all genes in control or SAC-defective cell lines will allow identification of genes that become essential in SAC-defective cells (one such example is indicated as gene X on the graph). [Figures adapted from Wang, T., et al., “Identification and characterization of essential genes in the human genome.” Science, 2015. 350(6264): p. 1096-101; and Wang, T., et al., “Gene Essentiality Profiling Reveals Gene Networks and Synthetic Lethal Interactions with Oncogenic Ras.” Cell, 2017. 168(5): p. 890-903 e15.]

FIGS. 14A-14C show the HEC-6 cell line displays altered Cdc20 isoform levels (FIG. 14A) DNA Sanger sequencing of CDC20 gene in HEC-6 cell line reveals the reported Q3stop mutation in at least one CDC20 allele. (FIG. 14B) HEC-6 cells express reduced levels of full-length Cdc20 protein and thus higher relative levels of the alternative Cdc20 isoforms, compared to control HeLa cells. (FIG. 14C) HEC-6 cells display reduced mitotic arrest duration compared to control HeLa cells when treated with 10 µM STLC.

### DETAILED DESCRIPTION OF THE INVENTION

### Some Definitions

The term “expression” refers to the cellular processes involved in producing RNA and proteins and as appropriate, secreting proteins, including where applicable, but not limited to, transcription, translation, folding, modification and processing. Expression products include RNA transcribed from a gene and polypeptides obtained by translation of mRNA transcribed from a gene.

The terms “subject” and “individual” are used interchangeably herein, and refer to an animal, for example, a human from whom cells can be obtained and/or to whom treatment, including prophylactic treatment is provided. For treatment of conditions or disease states which are specific for a specific animal such as a human subject, the term subject refers to that specific animal. The terms “non-human animals” and “non-human mammals” as used herein interchangeably, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### Methods of Screening for Anti-Cancer Agents

Some aspects of the present disclosure are directed to a method of screening for a candidate anti-cancer agent, comprising (a) providing a cell expressing a Cdc20 variant and resistant to an anti-mitotic drug; (b) contacting the cell with an anti-mitotic drug and a test agent; (c) determining if the test agent reduces mitotic slippage as compared to a control; and (d) identifying the test agent as a candidate anti-cancer agent if the test agent reduces mitotic slippage as compared to the control. In some embodiments, the cell is a cancer cell. In some embodiments, the Cdc20 variant comprises an N-terminal truncation.

As used herein, a “Cdc20 variant” is a protein with at least one difference from wild-type or full length Cdc20 (e.g., human wild-type Cdc20 of SEQ ID NO: 2). In some embodiments, the Cdc20 variant has an N-terminal truncation of at least the first 10, 15, 20, 25, 30, 31, 32, 33, 34, 35, 36, 37, 38, 39, 40, 41, 42, or 43 amino acids (e.g., of SEQ ID NO: 2 or a polypeptide with at least 90%, 91%, 92%, 93%, 94%, 95%, 96%, 97%, 98%, 99%, 99.5%, 99.9%, 99.95% identity to SEQ ID NO: 2). In some embodiments, the Cdc20 variant has one or more insertions, substitutions, and/or deletions located in the N-terminal region (e.g., of at least the first 10, 15, 20, 25, 30, 31, 32, 33, 34, 35, 36, 37, 38, 39, 40, 41, 42, or 43 amino acids (e.g., of SEQ ID NO: 2 or a polypeptide with at least 90%, 91%, 92%, 93%, 94%, 95%, 96%, 97%, 98%, 99%, 99.5%, 99.9%, 99.95% identity to SEQ ID NO: 2)). In some embodiments, the Cdc20 variant has an insertion, deletion, or modification in positions 27-34 of SEQ ID NO: 2 or a polypeptide with at least 90%, 91%, 92%, 93%, 94%, 95%, 96%, 97%, 98%, 99%, 99.5%, 99.9%, 99.95% identity to SEQ ID NO: 2, or the equivalent Box1 or BM1 motif residues. In some embodiments, the Cdc20 variant does not comprise positions 27-34 of SEQ ID NO: 2 (or a fragment thereof), or the equivalent Box1 or BM1 motif residues. In some embodiments, cells expressing only the Cdc20 variant are viable and can proliferate. In some embodiments, cells expressing only the Cdc20 variant have increased resistant to one or more anti-mitotic drugs (e.g., at least 1.2-fold, 1.5-fold, 2-fold, 3-fold, 4-fold, 5-fold, 6-fold, 10-fold, or more resistance to the one or more anti-mitotic drugs than an appropriate control). In some embodiments, cells expressing only the Cdc20 variant have a shortened period of mitotic arrest (e.g., an arrest period that is only about 10%, 20%, 30%, 40%, 50%, 60%, 70%, 80%, or 90% as long as a control cell contacted with the anti-mitotic drug). In some embodiments, the Cdc20 variant comprises residues 77-83 of SEQ ID NO: 2, or the equivalent C-box motif residues.

In some embodiments, the Cdc20 variant has a lower binding affinity for Mad2 (mitotic arrest deficient 2) than wild-type Cdc20 (e.g., human wild-type Cdc20 of SEQ ID NO: 2 or a polypeptide with at least 90%, 91%, 92%, 93%, 94%, 95%, 96%, 97%, 98%, 99%, 99.5%, 99.9%, 99.95% identity to SEQ ID NO: 2). In some embodiments, the Cdc20 variant has at least about 1.2-fold less, 1.5-fold less, 2-fold less, 3-fold less, 4-fold less, 5-fold less, 6-fold less, 10-fold less, or 20-fold less binding affinity that wild-type Cdc20 (e.g., human wild-type Cdc20 of SEQ ID NO: 2). In some embodiments, the Cdc20 variant is a Cdc20 variant disclosed herein. As used herein, “wild-type” Cdc20 is full length Cdc20.

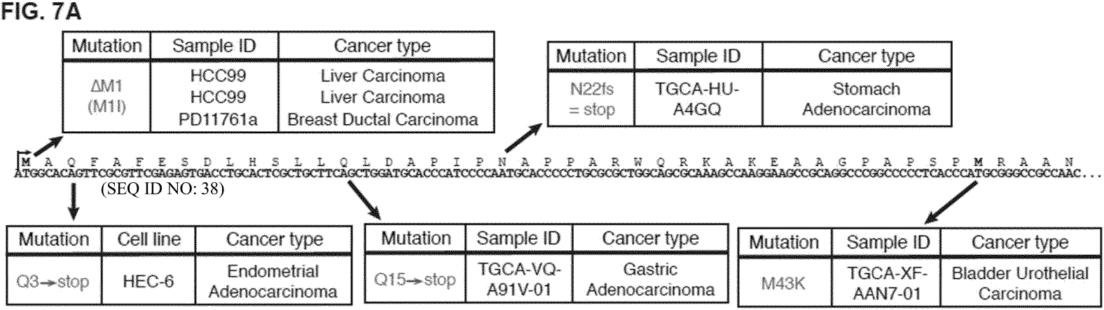

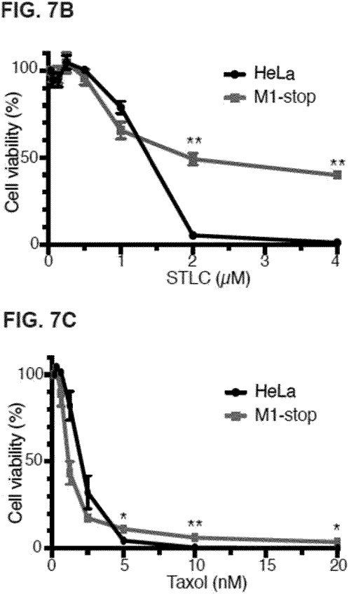

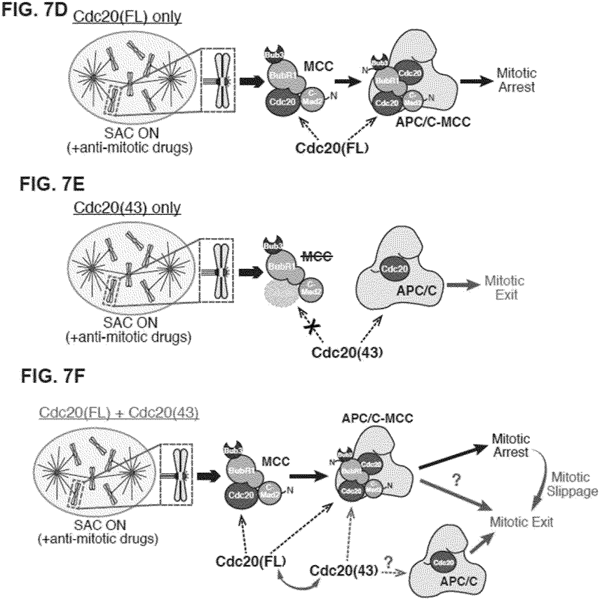

In some embodiments, the Cdc20 variant is a variant provided in FIG. 7A.

The anti-mitotic drug (i.e., anti-mitotic agent) is any compound that inhibits, prevents, or otherwise disrupts mitosis, is not limited, and may be any suitable anti-mitotic drug (e.g., an anti-cancer anti-mitotic drug). In some embodiments, the anti-mitotic drugs include, but are not limited to, taxanes, such as paclitaxel and docetaxel; maytansinoids, including maytansinol and maytansinol analogues modified in the aromatic ring or at other positions of the maytansinol molecule, such as various maytansinol esters, and DM1 and DM4; dolastatin 10, dolastatin 15, and auristatins, such as monomethyl auristatin E (MMAE) and monomethyl auristatin F (MMAF); vinca alkoloids, such as vinblastine and vincristine; and analogs and deriviatives thereof.

The taxanes are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

Maytansinoids are tubulin-binding agents that are potent anti-mitotics, causing cells to arrest in the G2/M phase of the cell cycle and ultimately leading to cell death. Maytansinoids are derivatives of the maytansine, a compound first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and maytansinol analogues have been reported. See U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, and Kawai et al (1984) Chem. Pharm. Bull. 3441-3451).

The auristatins are analogs of dolastatin 10 (a pentapeptide natural product), including monomethyl auristatin E (MMAE) and monomethyl auristatin F (MMAF). Molecules in this family inhibit tubulin polymerization. In general, the activities are 100-1,000 times more potent than doxorubicin. (Pettit, G. R., The dolastatins. Progress in the Chemistry of Organic Natural Products 70, 1-79, 1997).

The anti-mitotic agent is optionally conjugated to an antibody. Examples of anti-mitotic-antibody conjugates include, but are not limited to, trastuzumab-DM1 (Genentech/ImmunoGen, described in U.S. Pat. No. 7,097,840, incorporated by reference in its entirety herein), Trastuzumab-auristatin (Genentech/Seattle Genetics), Cantuzumab mertansine (huC242-DM1, SB-408075) (ImmunoGen), BB-10901 (huN901-DM1) (ImmunoGen), MLN2704(DM1) (Millennium Pharmaceuticals), Bivatuzumab mertansine (DM1) (Boehringer Ingelheim), huMy9-6-DM4 (AVE9633) (Sanofi-aventisc), huC242-DM4 (ImmunoGen), SGN-35 (Monomethyl auristatin) (Seattle Genetics), SGN-75 (Monomethyl auristatin) (Seattle Genetics), CR011-vcMIVIAE (Curagen/Seattle Genetics), and Trastuzumab-MCC-DM1 (T-DM1) (CAS Reg. No. 139504-50-0). In some embodiments, the anti-mitotic drug is a taxane or maytansinoid.

The test agent is not limited and may be any agent disclosed herein.

“Agent” is used herein to refer to any substance, compound (e.g., molecule), supramolecular complex, material, or combination or mixture thereof. In some aspects, an agent can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, peptide mimetics, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (-)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. Unless otherwise specified, the term “analog” as used herein refers to a structural analog. A structural analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey GF, et al., Chemical modification of siRNA. Curr. Protoc. Nucleic Acid Chem. 2009; 39:16.3.1-16.3.22; Crooke, ST (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long.

“Nucleic acid construct” refers to a nucleic acid that is generated by man and is not identical to nucleic acids that occur in nature, i.e., it differs in sequence from naturally occurring nucleic acid molecules and/or comprises a modification that distinguishes it from nucleic acids found in nature. A nucleic acid construct may comprise two or more nucleic acids that are identical to nucleic acids found in nature, or portions thereof, but are not found as part of a single nucleic acid in nature.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/ or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

The synthetic RNA can encode any suitable agent described herein. Synthetic RNAs, including modified RNAs are taught in WO 2017075406, which is herein incorporated by reference. In some embodiments, the agent is, or is encoded by, a synthetic RNA (e.g., modified mRNAs) conjugated to non-nucleic acid molecules. In some embodiments, the synthetic RNAs are conjugated to (or otherwise physically associated with) a moiety that promotes cellular uptake, nuclear entry, and/or nuclear retention (e.g., peptide transport moieties or the nucleic acids). In some embodiments, the synthetic RNA is conjugated to a peptide transporter moiety, for example a cell-penetrating peptide transport moiety, which is effective to enhance transport of the oligomer into cells.

In some embodiments, the oligonucleotide (e.g., synthetic RNA) is complementary to a region comprising a Cdc20 alternate translation start site and preferentially reduces translation of a Cdc20 isoform as compared to translation of the WT Cdc20 protein.

In some embodiments, the agent is a targetable nuclease (also referred to as a site specific nuclease or endonuclease) and, if appropriate, a guide molecule (e.g., one or more gRNA). The term “targetable nuclease” or “endonuclease” refers to a nuclease that can be programmed to produce site-specific DNA breaks, e.g., double-stranded breaks (DSBs), at a selected site in DNA. Such a site may be referred to as a “target site”. The target site can be selected by appropriate design of the targetable nuclease or by providing a guide molecule (e.g., a guide RNA) directs the nuclease to the target site. Examples of targetable nucleases include zinc finger nucleases (ZFNs), transcription activator- like effector nucleases (TALENs), and RNA-guided nucleases (RGNs) such as the Cas proteins of the CRISPR/Cas Type II system, and engineered meganucleases.

In some embodiments, the site-specific nuclease is catalytically inactive and is fused to an effector domain. As used herein an “effector domain” is a molecule (e.g., protein) that modulates the expression and/or activation of a genomic sequence (e.g., gene) or modifies the sequence or methylation of the genomic sequence. The effector domain may have methylation activity (e.g., DNA methylation activity). In some aspects, the effector domain targets one or both alleles of a gene. The effector domain can be introduced as a nucleic acid sequence and/or as a protein. In some aspects, the effector domain can be a constitutive or an inducible effector domain. In some aspects, a Cas (e.g., dCas) nucleic acid sequence or variant thereof and an effector domain nucleic acid sequence are introduced into the cell as a chimeric sequence. In some aspects, the effector domain is fused to a molecule that associates with (e.g., binds to) Cas protein (e.g., the effector molecule is fused to an antibody or antigen binding fragment thereof that binds to Cas protein). In some aspects, a Cas (e.g., dCas) protein or variant thereof and an effector domain are fused or tethered creating a chimeric protein and are introduced into the cell as the chimeric protein. In some aspects, the Cas (e.g., dCas) protein and effector domain bind as a protein-protein interaction. In some aspects, the Cas (e.g., dCas) protein and effector domain are covalently linked. In some aspects, the effector domain associates non-covalently with the Cas (e.g., dCas) protein. In some aspects, a Cas (e.g., dCas) nucleic acid sequence and an effector domain nucleic acid sequence are introduced as separate sequences and/or proteins. In some aspects, the Cas (e.g., dCas) protein and effector domain are not fused or tethered.

In some aspects, the effector domain is a DNA modifier. Specific examples of DNA modifiers include 5hmc conversion from 5mC such as Tetl (TetlCD); DNA demethylation by Tetl, ACID A, MBD4, Apobec1, Apobec2, Apobec3, Tdg, Gadd45a, Gadd45b, ROS1; DNA methylation by Dnmtl, DNMT3A, Dnmt3b, CpG Methyltransferase M.SssI, and/or M.EcoHK31I. In specific aspects, an effector domain is DNMT3A. In some aspects, the effector domain is the C-terminal domain of DNMT3A (i.e., DNMT3A-C). In some aspects, the DNMT3A-C effector domain is complexed with the C-terminal portion of DNMT3L (DNMT3L-C). In some aspects, a chimeric protein comprising DNMT3A-C and DNMT3L-C (sometimes referred to herein as DNMT3A-3L) is used for the effector domain. In some aspects, DNMT3A-3L is a single chain fusion protein as provided in Siddique, et al. (2013) incorporated herein by reference in its entirety. In some embodiments, the effector domain is DNMT3A-3L without the 5′ NLS. In some embodiments, dCas9 is fused to DNMT3A-3L or DNMT3A-3L without the 5′ NLS.

DNA methylation is established by two de novo DNA methyltransferases (DNMT3A/B), and is maintained by DNMT1 (Smith and Meissner, (2013). DNA methylation: roles in mammalian development. Nature reviews Genetics 14, 204-220). Gene activation during development is associated with demethylation of promoter and enhancer sequences. In addition, demethylation can be achieved through oxidation of the methyl group by TET (ten-eleven translocation) dioxygenases to form 5-hydroxymethylcytosine (5-hmC), and then restoration into unmodified cytosines by either DNA replication-dependent dilution or DNA glycosylase-initiated base excision repair (BER), a process termed as active demethylation and proposed to operate during specific developmental stages such as preimplantation embryos or in post-mitotic neurons.

The cell for screening is not limited and may be any suitable cell. In some embodiments, the cell is a cancer cell or a cancer cell line cell. In some embodiments, the cancer is selected from acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g.,2 adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi′ s sarcoma, multiple idiopathic hemorrhagic sarcoma); endometrial cancer (e.g., uterine cancer, uterine sarcoma); esophageal cancer (e.g., adenocarcinoma of the esophagus, Barrett′ s adenocarinoma); Ewing′ s sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström′ s macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms’ tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis ); neuroendocrine cancer (e.g., gastroenteropancreatic neuroendoctrine tumor (GEP-NET), carcinoid tumor); osteosarcoma (e.g.,bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget′ s disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma ( e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget’s disease of the vulva).

In some embodiments, the cancer is liver carcinoma, breast ductal carcinoma, stomach adenocarcinoma, endometrial adenocarcinoma, gastric adenocarcinoma, or bladder urothelial carcinoma.

In some embodiments, the cell is resistant to an anti-mitotic drug (e.g., has a higher rate of mitotic slippage in the presence of the anti-mitotic drug than a suitable non-resistant cell or other control). In some embodiments, the cell is a cancer cell that has developed resistance to a anti-mitotic drug after treatment with the drug.

As used herein, “mitotic slippage” refers to cells exiting mitosis without proper chromosome segregation. In some embodiments, a test agent is identified as a candidate anti-cancer agent if the test agent reduces mitotic slippage by at least about 10%, 20%, 30%, 40%, 50%, 60%, 70%, 80%, 90%, or more as compared to a suitable control (e.g., a cell contacted with the anti-mitotic drug and not the agent). In some embodiments, a test agent is identified as a candidate anti-cancer agent if the test agent increases the mitotic arrest duration by at least about 10%, 20%, 30%, 40%, 50%, 60%, 70%, 80%, 90%, or more as compared to a suitable control (e.g., a cell contacted with the anti-mitotic drug and not the agent).

In some embodiments, mitotic slippage is detected by increased survival and/or proliferation in the presence of the anti-mitotic drug. In certain embodiments of any method described herein, the survival or proliferation of cells, e.g., test cells and/or control cells, is determined by an assay selected from: a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, and a nuclear fragmentation assay. Exemplary assays include BrdU, EdU, or H3-Thymidine incorporation assays; DNA content assays using a nucleic acid dye, such as Hoechst Dye, DAPI, actinomycin D, 7-aminoactinomycin D or propidium iodide; cellular metabolism assays such as AlamarBlue, MTT, XTT, and CellTitre Glo; nuclear fragmentation assays; cytoplasmic histone associated DNA fragmentation assay; PARP cleavage assay; TUNEL staining; and Annexin staining. In some embodiments, gene expression analysis (e.g., microarray, cDNA array, quantitative RT-PCR, RNAse protection assay, RNA-Seq) may be used to measure the expression of genes whose products mediate or are correlated with cell cycle, cell survival (or cell death, e.g., apoptosis), and/or cell proliferation, as an indication of the effect of an agent on cell viability or proliferation. Alternately or additionally, expression of proteins encoded by such genes may be measured. In other embodiments, the activity of a gene, such as those disclosed herein, can be assayed in a compound screen. In some embodiments, cells are modified to comprise an expression vector that includes a regulatory region of a gene whose products mediate or are correlated with cell cycle, cell survival (or cell death), and/or cell proliferation operably linked to a sequence that encodes a reporter gene product (e.g., a luciferase enzyme), wherein expression of the reporter gene is correlated with transcriptional activity of the gene. In such embodiments, assessment of reporter gene expression (e.g., luciferase activity) provides an indirect method for assessing cell survival or proliferation. Those of ordinary skill in the art are aware of genes whose products mediate or are correlated with cell cycle, cell survival (or cell death), and/or cell proliferation.

In various embodiments the number of test agents is at least 10; 100; 1000; 10,000; 100,000; 250,000; 500,000 or more. In some embodiments test agents are tested in individual vessels, e.g., individual wells of a multiwell plate (sometimes referred to as microwell or microtiter plate or dish). In some embodiments a multiwell plate of use in performing an assay or culturing or testing cells or agents has 6, 12, 24, 96, 384, or 1536 wells. Cells (test cells and/or control cells) can be contacted with one or more test agents for varying periods of time and/or at different concentrations. In certain embodiments cells are contacted with test agent(s) for between 1 hour and 20 days, e.g., for between 12 and 48 hours, between 48 hours and 5 days, e.g., about 3 days, between 2 and 5 days, between 5 days and 10 days, between 10 days and 20 days, or any intervening range or particular value. Cells can be contacted with a test agent during all or part of a culture period. Cells can be contacted transiently or continuously. Test agents can be added to culture media at the time of replenishing the media and/or between media changes. If desired, test agent can be removed prior to assessing growth and/or survival. In some embodiments a test agent is tested at 1, 2, 3, 5, 8, 10 or more concentrations. Concentrations of test agent may range, for example, between about 1 nM and about 100 µM. For example, concentrations 1 nM, 10 nM, 50 nM, 100 nM, 500 nM, 1 µM, 5 µM, 10 µM, 50 µM, 100 µM (or any subset of the foregoing) may be used.

In some embodiments of any aspect or embodiment in the present disclosure relating to cells, a population of cells, cell sample, or similar terms, the number of cells is between 10 and 10¹³ cells. In some embodiments the number of cells may be at least about 10³, 10⁴, 10⁵, 10⁶, 10⁷, 10⁸, 10⁹, 10¹⁰, 10¹¹, 10¹² cells, or more. In some embodiments, the number of cells is between 10⁵ and 10¹² cells, e.g., at least 10⁶, 10⁷, 10⁸, 10⁹, 10¹⁰, 10¹¹, up to about 10¹² or about 10¹³. In some embodiments a screen is performed using multiple populations of cells and/or is repeated multiple times. In some embodiments, the number of cells is between 10⁵ and 10¹² cells, e.g., at least 10⁶, 10⁷, 10⁸, 10⁹, 10¹⁰, 10¹¹, up to about 10¹². In some embodiments smaller numbers of cells are of use, e.g., between 1 - 10⁴ cells. In some embodiments a population of cells is contained in an individual vessel, e.g., a culture vessel such as a culture plate, flask, or well. In some embodiments a population of cells is contained in multiple vessels. In some embodiments two or more cell populations are pooled to form a larger population.

In some embodiments, each of one or more test cells is contacted with a different concentration of, and/or for a different duration with, a test agent than at least one other test cell; and/or each of the one or more control cells is contacted with a different concentration of, and/or for a different duration with, the test agent than at least one other control cell.

In some embodiments, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg RP. Design and implementation of high-throughput screening assays. Methods Mol Biol., 565:1-32, 2009 and/or An WF & Tolliday NJ., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hÿser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminate such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a non-target molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. An analog that has one or more improved properties may be identified and used in a composition or method described herein. In some embodiments a molecular target of a hit compound is identified or known. In some embodiments, additional compounds that act on the same molecular target may be identified empirically (e.g., through screening a compound library) or designed.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics.

Once a candidate agent is identified, additional agents, e.g., analogs, may be generated based on it. An additional agent, may, for example, have increased cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

### Methods of Treatment With Anti-Mitotic Drugs

Some aspects of the present disclosure are directed to a method of determining if a subject with cancer is a candidate for anti-mitotic drug therapy, comprising (a) measuring the expression level of one or more Cdc20 variants in the cancer, and (b) determining that the subject is a candidate for anti-mitotic drug therapy if the expression level is below a threshold. In some embodiments, the method further comprises administering the anti-mitotic drug therapy to the subject determined to be a candidate for anti-mitotic drug therapy.

Some aspects of the present disclosure are directed to a method of determining if a subject with cancer is a candidate for anti-mitotic drug therapy, comprising (a) measuring the expression level of one or more Cdc20 variants and the expression level of Cdc20 wild-type in the cancer, and (b) determining that the subject is a candidate for anti-mitotic drug therapy if the ratio of the expression level of the one or more Cdc20 variants to Cdc20 wild-type is below a threshold. In some embodiments, the method further comprises administering the anti-mitotic drug therapy to the subject determined to be a candidate for anti-mitotic drug therapy.

Some aspects of the present disclosure are directed to a method of treating a subject in need thereof with and anti-mitotic drug therapy, comprising (a) identifying a subject having an expression level of one or more Cdc20 variants resistant to anti-mitotic drug therapy below a threshold, and (b) administering the anti-mitotic drug therapy to the subject.

Some aspects of the present disclosure are directed to a method of treating a subject in need thereof with and anti-mitotic drug therapy, comprising (a) identifying a subject having a ratio of an expression level of one or more Cdc20 variants resistant to anti-mitotic drug therapy to an expression level of Cdc20 wild-type below a threshold, and (b) administering the anti-mitotic therapy to the subject.

The subject is not limited and may be any subject disclosed herein. In some embodiments, the subject is a human with cancer.

The anti-mitotic drug is not limited and may be any anti-mitotic drug described herein. In some embodiments, a combination of anti-mitotic drugs are administered to the subject.

The Cdc20 variant is not limited and may be any Cdc20 variant that provides resistance to an anti-mitotic drug. In some embodiments, the Cdc20 variant has an insertion, deletion, or substitution in a region of the Cdc20 protein corresponding to residues in positions 27-34 of SEQ ID NO: 2, or the equivalent Box1 or BM1 motif residues. In some embodiments, the Cdc20 variant comprises an N-terminal deletion as described herein.

The threshold expression level for Cdc20 variant below which the subject is a candidate for anti-mitotic therapy is any suitable threshold. In some embodiments, the threshold is empirically determined.

The threshold ratio of an expression level of one or more Cdc20 variants resistant to anti-mitotic drug therapy to an expression level of Cdc20 wild-type below which the subject is a candidate for anti-mitotic therapy is any suitable ratio. In some embodiments, the ratio is empirically determined. In some embodiments, the threshold ratio of an expression level of one or more Cdc20 variants resistant to anti-mitotic drug therapy to an expression level of Cdc20 wild-type below which the subject is a candidate for anti-mitotic therapy is about 1:1, 1:1.1, 1:1.2, 1:1.3, 1:1.4, 1:1.5, 1:1.6, 1:1.7, 1:8.1, 1:1.9, 1:2, 1:2.1, 1:2.2, 1:2.3, 1:2.4, 1:2.5, 1:2.6, 1:2.7, 1:2.8, 1:2.9, 1:3, 1:3.5, 1:4, 1:4.5, 1:5, 1:5.5, 1:6, 1:6.5, 1:7, 1:7.5, 1:8, 1:9, 1:10, 1:15, 1:20, 1:30, 1:40, 1:50, 1:75, 1:100, 1:200, 1:500, or more.

Methods of detecting the expression levels of Cdc20 variants and Cdc20 wild-types are not limited and may be any suitable method. In some embodiments, the expression levels are determined by immunohistochemistry. In many embodiments, an immunological method or other affinity-based method is used. In general, immunological detection methods involve detecting specific antibody-antigen interactions in a sample such as a tissue section or cell sample. The sample is contacted with an antibody that binds to the target antigen of interest. The antibody is then detected using any of a variety of techniques. In some embodiments, the antibody that binds to the antigen (primary antibody) or a secondary antibody that binds to the primary antibody has been tagged or conjugated with a detectable label. In some embodiments a label-free detection method is used. A detectable label may be, for example, a fluorescent dye (e.g., a fluorescent small molecule) or quencher, colloidal metal, quantum dot, hapten, radioactive atom or isotope, or enzyme (e.g., peroxidase). It will be appreciated that a detectable label may be directly detectable or indirectly detectable. For example, a fluorescent dye would be directly detectable, whereas an enzyme may be indirectly detectable, e.g., the enzyme reacts with a substrate to generate a directly detectable signal. Numerous detectable labels and strategies that may be used for detection, e.g., immunological detection, are known in the art. Exemplary immunological detection methods include, e.g., immunohistochemistry (IHC); enzyme-linked immunosorbent assay (ELISA), bead-based assays such as the Luminex® assay platform (Invitrogen), flow cytometry, protein microarrays, surface plasmon resonance assays (e.g., using BiaCore technology), microcantilevers, immunoprecipitation, immunoblot (Western blot), etc. IHC generally refers to immunological detection of an antigen of interest (e.g., a cellular constituent) in a tissue sample such as a tissue section. As used herein, IHC is considered to encompass immunocytochemistry (ICC), which term generally refers to the immunological detection of a cellular constituent in isolated cells that essentially lack extracellular matrix components and tissue microarchitecture that would typically be present in a tissue sample. Traditional ELISA assays typically involve use of primary or secondary antibodies that are linked to an enzyme, which acts on a substrate to produce a detectable signal (e.g., production of a colored product) to indicate the presence of antigen or other analyte. IHC generally refers to the immunological detection of a tissue or cellular constituent in a tissue or cell sample comprising substantially intact (optionally permeabilized) cells. As used herein, the term “ELISA” also encompasses use of non-enzymatic reporters such as fluorogenic, electrochemiluminescent, or real-time PCR reporters that generate quantifiable signals. It will be appreciated that the term “ELISA” encompasses a number of variations such as “indirect”, “sandwich”, “competitive”, and “reverse” ELISA.

Methods of administering the anti-mitotic drug therapy are not limited and may be any suitable method. In some embodiments, the anti-mitotic drug is administered via a route selected from the group consisting of intravenously, subcutaneously, intra-arterially, intrathecally, and intra-muscularly.

### Methods of Treating Anti-Mitotic Drug Resistant Cancer

Some aspects of the present disclosure are directed to a method of inhibiting a cancer cell expressing a Cdc20 variant and resistant to an anti-mitotic drug comprising contacting the cancer cell with an agent that reduces the expression or activity of the Cdc20 variant resistant to an anti-mitotic drug or wherein the modification increases the expression or activity of wild-type Cdc20. In some aspects of the present disclosure, the method is used to treat anti-mitotic drug resistant cancer. In some embodiments, the agent is administered to a subject before or simultaneously with the anti-mitotic drug.

As used herein, the “activity” of the Cdc20 variant is the promotion of mitotic slippage.

The cancer cell is not limited and may be any cell described herein. In some embodiments, the cancer is liver carcinoma, breast ductal carcinoma, stomach adenocarcinoma, endometrial adenocarcinoma, gastric adenocarcinoma, or bladder urothelial carcinoma. As used herein, “resistant to an anti-mitotic drug” means that the cancer is partially (e.g., at least 2-fold more resistant than an appropriate control cell) or completely refractory to therapy with the anti-mitotic drug. The Cdc20 variant is not limited and may be any Cdc20 variant (e.g., as disclosed herein) that confers resistance to anti-mitotic therapy. In some embodiments, the Cdc20 variant has an insertion, deletion, or substitution in a region of the Cdc20 protein corresponding to residues in positions 27-34 of SEQ ID NO: 2, or the equivalent Box1 or BM1 motif residues. In some embodiments, the Cdc20 variant comprises an N-terminal deletion as described herein.

The agent is not limited and may be any agent described herein. In some embodiments, the agent inhibits the binding of the Cdc20 variant with APC/C. In some embodiments, the agent inhibits the expression of the Cdc20 variant. In some embodiments, the agent increases the expression or activity of Cdc20 wild-type or a Cdc20 variant not resistant to the anti-mitotic drug.

In some embodiments, the agent increases the mitotic arrest duration by at least about 10%, 20%, 30%, 40%, 50%, 60%, 70%, 80%, 90%, or more as compared to a suitable control. In some embodiments, agent reduces mitotic slippage by at least about 10%, 20%, 30%, 40%, 50%, 60%, 70%, 80%, 90%, or more as compared to a suitable control.

In some embodiments, the agent is the Cdc20 wild-type or the Cdc20 variant not resistant to the anti-mitotic drug, or a nucleotide sequence coding for the same. In some embodiments, the agent comprises residues 1-42 of SEQ ID NO: 2, or a functional fragment thereof.

In some embodiments, the cancer cell is inhibited in vivo in a subject. The subject is not limited and may be any subject described herein. In some embodiments, the subject is a human with cancer. In some embodiments, the agent is administered to the subject. In some embodiments, the subject is also administered the anti-mitotic drug. Methods of administration are not limited and may be any method disclosed herein.

Some aspects of the present disclosure are directed to a method of inhibiting a cancer cell expressing a Cdc20 variant and resistant to an anti-mitotic drug comprising contacting the cancer cell with an endonuclease and modifying the genome of the cancer cell, wherein the modification reduces or eliminates the expression of a Cdc20 variant resistant to an anti-mitotic drug or wherein the modification increases the expression of wild-type Cdc20.

The endonuclease (i.e., targetable nuclease) is not limited and may be any suitable endonuclease. In some embodiments, the endonuclease is a zinc finger nuclease (ZFN), transcription activator- like effector nuclease (TALEN), or a RNA-guided nuclease (RGN) such as the Cas proteins of the CRISPR/Cas Type II system, and an engineered meganuclease. In some embodiments, the endonuclease is a Cas9 nuclease and the cancer cell is further contacted with one or more gRNA.

In some embodiments, the modification eliminates one or more Cdc20 alternate translation start sites (e.g., by contact with a Cas protein and one or more gRNA targeting the alternate translation start sites). In some embodiments, the alternate translation start site is located at positions 127-129 of SEQ ID NO: 1. In some embodiments, the alternate translation start site is located at positions 262-264 of SEQ ID NO: 1.

In some embodiments, the modification increases translation from the wild-type translation start site. In some embodiments, the modification increases translation of full length wild-type protein. In some embodiments, the modification introduces a substitution in the wild-type translation start site (e.g., positions 9-11 of SEQ ID NO: 1) or modifies a promoter binding site. In some embodiments, the modification provides a consensus Kozak sequence.

In some embodiments, the modification removes a mutation decreasing expression of WT Cdc20. In some embodiments, the mutation is a stop codon mutation located between the WT translation start site and one or more of the alternate translation start sites. In some embodiments, the mutation converts a glutamine codon to a stop codon. In some embodiments, the mutation converts CAG (Q, glutamine) to TAG (stop codon) and results in a dipeptide (Met-Ala), rather than full-length Cdc20 protein (e.g., of SEQ ID NO: 2 or a polypeptide with at least 90%, 91%, 92%, 93%, 94%, 95%, 96%, 97%, 98%, 99%, 99.5%, 99.9%, 99.95% identity to SEQ ID NO: 2).

In some embodiments, the cancer cell is inhibited in vivo in a subject. The subject is not limited and may be any subject described herein. In some embodiments, the subject is a human with cancer. In some embodiments, the agent is administered to the subject. In some embodiments, the subject is administered the anti-mitotic drug after the cell is modified. Methods of administration are not limited and may be any method disclosed herein.

Generally, treatment of a subject can include a single treatment or, in many cases, can include a series of treatments. A pharmaceutical composition can be administered at various intervals and over different periods of time as required, e.g., multiple times per day, daily, every other day, once or more a week for between about 1 to 10 weeks, between 2 to 8 weeks, between about 3 to 7 weeks, about 4, 5, or 6 weeks, etc. It will be appreciated that multiple cycles of administration may be performed. Numerous variations are possible. The skilled artisan will appreciate that certain factors can influence the dosage and timing required to effectively treat a subject, including but not limited to the severity of the disease or disorder, previous treatments, the general health and/or age of the subject, and other diseases present.

### Compositions

Some aspects of the present disclosure are directed to a composition (e.g., pharmaceutical composition) comprising an agent disclosed herein. In some embodiments, the agent reduces the expression or activity (e.g., promoting mitotic slippage) of the Cdc20 variant.

Some aspects of the present disclosure are directed to a composition comprising a Cas protein or a nucleotide sequence encoding a Cas protein and one or more gRNA or a nucleotide sequence encoding one or more gRNA targeting a Cdc20 alternate translation start site, wherein the composition is capable of eliminating the alternate translation start site. In some embodiments, the composition comprises one or more viruses capable of transducing a nucleotide sequence encoding a Cas protein and one or more gRNA. In some embodiments, the composition comprises a liposome, a lipid-based particle, a nanoparticle, a microparticle, a polymeric particle, or other delivery vehicle delivering a Cas protein or a nucleotide sequence encoding a Cas protein and one or more gRNA or a nucleotide sequence encoding one or more gRNA targeting a Cdc20 alternate translation start site.

The Cas protein is not limited. The Clustered Regularly Interspaced Short Palindromic Repeats (CRISPR) Type II system is a bacterial adaptive immune system that has been modified for use as an RNA-guided endonuclease technology for genome engineering. The bacterial system comprises two endogenous bacterial RNAs called crRNA and tracrRNA and a CRISPR-associated (Cas) nuclease, e.g., Cas9. The tracrRNA has partial complementarity to the crRNA and forms a complex with it. The Cas protein is guided to the target sequence by the crRNA/tracrRNA complex, which forms an RNA/DNA hybrid between the crRNA sequence and the complementary sequence in the target. For use in genome modification, the crRNA and tracrRNA components are often combined into a single chimeric guide RNA (sgRNA or gRNA) in which the targeting specificity of the crRNA and the properties of the tracrRNA are combined into a single transcript that localizes the Cas protein to the target sequence so that the Cas protein can cleave the DNA. The gRNA often comprises an approximately 20 nucleotide guide sequence complementary or homologous to the desired target sequence followed by about 80 nt of hybrid crRNA/tracrRNA. One of ordinary skill in the art appreciates that the guide RNA need not be perfectly complementary or homologous to the target sequence. For example, in some embodiments it may have one or two mismatches. The genomic sequence which the gRNA hybridizes is typically flanked on one side by a Protospacer Adjacent Motif (PAM) sequence although one of ordinary skill in the art appreciates that certain Cas proteins may have a relaxed requirement for a PAM sequence. The PAM sequence is present in the genomic DNA but not in the gRNA sequence. The Cas protein will be directed to any DNA sequence with the correct target sequence and PAM sequence. The PAM sequence varies depending on the species of bacteria from which the Cas protein was derived. Specific examples of Cas proteins include Cas1, Cas2, Cas3, Cas4, Cas5, Cas6, Cas7, Cas8, Cas9 and Cas10. In some embodiments, the Cas protein comprises a Cas9 protein. For example, Cas9 from Streptococcus pyogenes (Sp), Neisseria meningitides, Staphylococcus aureus, Streptococcus thermophiles, or Treponema denticola may be used. The PAM sequences for these Cas9 proteins are NGG, NNNNGATT, NNAGAA, NAAAAC, respectively. In some embodiments, the Cas9 is from Staphylococcus aureus (saCas9). In some embodiments, the Cas9 is a small Cas9 ortholog from Staphylococcus auricularis (SauriCas9), which recognizes a simple NNGG PAM, displays high activity for genome editing, and is compact enough to be packaged into an AAV for genome editing. In some embodiments, the Cas protein is Campylobacter jejuni (CjCas9), Neisseria meningitidis Cas9 (NmeCas9), Cas12b (see, Strecker et al., Nat Commun. 2019 Jan 22;10(1):212), or CasX (see, Nature. 2019 Feb 4. pii: 10.1038/s41586-019-0908-x. doi: 10.1038/s41586-019-0908-x).

A number of engineered variants of the Cas proteins have been developed and may be used in certain embodiments. For example, engineered variants of Cas9 are known in the art. Furthermore, it will be understood that a biologically active fragment or variant can be used. Other variations include the use of hybrid site specific nucleases. For example, in CRISPR RNA-guided FokI nucleases (RFNs) the FokI nuclease domain is fused to the amino-terminal end of a catalytically inactive Cas9 protein (dCas9) protein.

RFNs act as dimers and utilize two guide RNAs (Tsai, QS, et al., Nat Biotechnol. 2014; 32(6): 569-576). Site-specific nucleases that produce a single-stranded DNA break are also of use for genome editing. Such nucleases, sometimes termed “nickases” can be generated by introducing a mutation (e.g., an alanine substitution) at key catalytic residues in one of the two nuclease domains of a site specific nuclease that comprises two nuclease domains (such as ZFNs, TALENs, and Cas proteins). Examples of such mutations include D10A, N863A, and H840A in SpCas9 or at homologous positions in other Cas9 proteins. A nick can stimulate HDR at low efficiency in some cell types. Two nickases, targeted to a pair of sequences that are near each other and on opposite strands can create a single-stranded break on each strand (“double nicking” ), effectively generating a DSB, which can optionally be repaired by HDR using a donor DNA template (Ran, F. A. et al. Cell 154, 1380-1389 (2013). In some embodiments, the Cas protein is a SpCas9 variant. In some embodiments, the SpCas9 variant is a R661A/Q695A/Q926A triple variant or a N497A/R661A/Q695A/ Q926A quadruple variant. See Kleinstiver et al., “High-fidelity CRISPR- Cas9 nucleases with no detectable genome-wide off-target effects,” Nature, Vol. 529, pp. 490-495 (and supplementary materials) (2016); incorporated herein by reference in its entirety. In some embodiments, the Cas protein is C2c1, a class 2 type V-B CRISPR-Cas protein. See Yang et al., “PAM-Dependent Target DNA Recognition and Cleavage by C2c1 CRISPR-Cas Endonuclease,” Cell, Vol. 167, pp. 1814- 1828 (2016); incorporated herein by reference in its entirety. In some embodiments, the Cas protein is one described in US 20160319260 “Engineered CRISPR-Cas9 nucleases with Altered PAM Specificity,” incorporated herein by reference.

In some aspects, the composition comprises a modified or synthetic mRNA encoding a Cas protein. In some embodiments, the modified or synthetic mRNA comprises one or more modifications that stabilize the mRNA or provide other improvements over naturally occurring mRNA (e.g., increased cellular uptake). Examples of modified or synthetic mRNA are described in Warren et al. (Cell Stem Cell 7(5):618-30, 2010, Mandal PK, Rossi DJ. Nat Protoc. 2013 8(3):568-82, US Pat. Pub. No. 20120046346 and/or PCT/US2011/032679 (WO/2011/130624). mRNA is also discussed in R.E. Rhoads (Ed.), “Synthetic mRNA: Production, Introduction Into Cells, and Physiological Consequences,” Series: Methods in Molecular Biology, Vol. 1428. Additional examples are found in numerous PCT and US applications and issued patents to Moderna Therapeutics, e.g., PCT/US2011/046861; PCT/US2011/054636, PCT/US2011/054617, USSN 14/390,100 (and additional patents and patent applications mentioned in these.)

In some embodiments, the composition is capable of introducing an insertion, deletion, or substitution into the alternate translation start site.

Some aspects of the present disclosure are directed to a composition (e.g., pharmaceutical composition) comprising one or more viruses transducing nucleotide sequences encoding a Cas9 nuclease and one or more gRNA targeting a Cdc20 alternate translation start site (i.e., as an agent), wherein the composition is capable of eliminating or reducing translation from the alternate translation start site. In some embodiments, the composition is capable of introducing an insertion, deletion, or substitution into the alternate translation start site.

Some aspects of the present disclosure are directed to a composition (e.g., pharmaceutical composition) comprising one or more viruses transducing nucleotide sequences encoding a Cas9 nuclease and one or more gRNA targeting a region enhancing translation of wild-type Cdc20 (i.e., as an agent). In some embodiments, the composition is capable of introducing an insertion, deletion, or substitution into the wild-type Cdc20 translation start site.

In addition to the active agent(s), the compositions typically comprise a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

A pharmaceutically-acceptable carrier employed in conjunction with the compounds described herein is used at a concentration or amount sufficient to provide a practical size to dosage relationship. The pharmaceutically-acceptable carriers, in total, may, for example, comprise from about 60% to about 99.99999% by weight of the pharmaceutical compositions, e.g., from about 80% to about 99.99%, e.g., from about 90% to about 99.95%, from about 95% to about 99.9%, or from about 98% to about 99%.

Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

Pharmaceutically acceptable compositions can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof in certain embodiments. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore, it will be appreciated that agents may be modified, e.g., with targeting moieties, moieties that increase their uptake, biological half-life (e.g., pegylation), etc.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, agents may be administered directly to a tissue, e.g., a tissue in which the cancer cells are found or one in which a cancer is likely to arise. Direct tissue administration may be achieved by direct injection. The agents may be administered once, or alternatively they may be administered in a plurality of administrations. If administered multiple times, the agents may be administered via different routes. For example, the first (or the first few) administrations may be made directly into the affected tissue while later administrations may be systemic.

For oral administration, compositions can be formulated readily by combining the active agent(s) with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer’s dextrose, dextrose and sodium chloride, lactated Ringer’s, or fixed oils. Intravenous vehicles include fluid and nutrient replenishers, electrolyte replenishers (such as those based on Ringer’s dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In certain embodiments, the vehicle is a biocompatible microparticle or implant that is suitable for implantation into the mammalian recipient. Exemplary bioerodible implants that are useful in accordance with this method are described in PCT International Application Publication No. WO 95/24929, entitled “Polymeric Gene Delivery System”, which reports on a biodegradable polymeric matrix for containing a biological macromolecule. The polymeric matrix may be used to achieve sustained release of the agent in a subject. In some embodiments, an agent described herein may be encapsulated or dispersed within a biocompatible, preferably biodegradable polymeric matrix. The polymeric matrix may be in the form of a microparticle such as a microsphere (wherein the agent is dispersed throughout a solid polymeric matrix) or a microcapsule (wherein the agent is stored in the core of a polymeric shell). Other forms of polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents may be delivered using the bio-erodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H.S. Sawhney, C.P. Pathak and J.A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the peptide, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. 5,075,109. Delivery systems also include non-polymer systems that are: lipids including sterols such as cholesterol, cholesterol esters and fatty acids or neutral fats such as mono- di- and triglycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. Liposomes, for example, which may comprise phospholipids or other lipids, are nontoxic, physiologically acceptable carriers that may be used in some embodiments. Liposomes can be prepared according to methods known to those skilled in the art. In some embodiments, for example, liposomes may be prepared as described in U.S. Pat. No. 4,522,811. Liposomes, including targeted liposomes, pegylated liposomes, and polymerized liposomes, are known in the art (see, e.g., Hansen C B, et al., Biochim Biophys Acta. 1239(2):133-44, 1995; Torchilin V P, et al., Biochim Biophys Acta, 1511(2):397-411, 2001; Ishida T, et al., FEBS Lett. 460(1):129-33, 1999). In some embodiments, a lipid-containing particle may be prepared as described in any of the following PCT application publications, or references therein: WO/2011/127255; WO/2010/080724; WO/2010/021865; WO/2010/014895; WO2010147655.

In some embodiments, it may be advantageous to formulate oral or parenteral compositions in dosage unit form for ease of administration and uniformity of dosage. Unit dosage form as used herein refers to physically discrete units suited as unitary dosages for the subject to be treated; each unit containing a predetermined quantity of active compound calculated to produce the desired therapeutic effect in association with the required pharmaceutical carrier.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

### Summary

Anti-mitotic drugs activate the Spindle Assembly Checkpoint (SAC) to induce a prolonged mitotic arrest. Human cell lines display widely varying durations of mitotic arrest, with important consequences for cell death and drug efficacy. However, the molecular features that govern mitotic arrest timing remain unclear. Here it is demonstrated that cells express multiple translational isoforms of the APC/C coactivator Cdc20. These alternative Cdc20 isoforms initiate at downstream start sites resulting in truncated Cdc20 proteins that are resistant to SAC-mediated inhibition and allow mitotic exit even in the presence of mitotic perturbations. Targeted changes that modify the relative levels of Cdc20 isoforms alter mitotic arrest duration and cellular viability upon treatment with anti-mitotic drugs. Translational regulatory mechanisms that impact the expression of the Cdc20 isoforms is also identified. This work reveals a mechanism to control mitotic arrest duration based on translational regulation of Cdc20 with important implications for the diagnosis and treatment of human cancers.

### Introduction

Anti-mitotic drugs that disrupt mitotic progression are widely used as cancer chemotherapy agents. For example, microtubule toxins such as paclitaxel (taxol), other taxanes, and vinca alkaloids, are first-line chemotherapeutics for the treatment of breast cancer, ovarian cancer, and other types of cancer. Anti-mitotic drugs disrupt the mitotic spindle in dividing cells, activating a regulatory pathway known as the Spindle Assembly Checkpoint (SAC) to induce prolonged mitotic arrest and ultimately cell death. The SAC is a surveillance mechanism that controls mitotic progression by delaying anaphase onset if even a single kinetochore fails to form proper microtubule attachments. The mechanisms by which anti-mitotic drugs target and disrupt mitotic processes are well-established. However, despite similar requirements for the execution of mitosis, human cell lines vary widely in their responses to such chemotherapeutics and their abilities to escape from the prolonged mitotic arrest induced by treatment with these compounds. As mitotic arrest behavior has important implications for the efficacy of anti-mitotic drugs, it is critical to define the molecular players and features that govern mitotic arrest duration.

The presence of improper kinetochore-microtubule attachments triggers Spindle Assembly Checkpoint signaling. The molecular players that comprise the SAC are conserved across eukaryotes and include Mad1, Mad2, BubR1/Mad3, Bub1, Bub3, and Mps1. The recruitment of SAC components to unattached kinetochores culminates in the formation of the checkpoint effector, the mitotic checkpoint complex (MCC). The MCC consists of three SAC proteins (Mad2, BubR1/Mad3, and Bub3) in complex with the essential cell cycle protein Cdc20. Cdc20 is also a critical co-activator of the anaphase-promoting complex, also known as the cyclosome (APC/C), an E3 ubiquitin ligase that directs the ubiquitination and degradation of mitotic substrates to promote chromosome segregation and mitotic exit. The MCC negatively regulates Cdc20 to inhibit APC/C function and arrest cells in mitosis. The association of Cdc20 with Mad2 at unattached kinetochores is the rate-limiting step in MCC assembly and proper SAC signaling.

The opposing roles of Cdc20 as the target of the SAC and as an essential APC/C cofactor required for mitotic progression position this essential cell cycle protein at the intersection of multiple signaling events that regulate cell division in eukaryotes. However, the molecular mechanisms that regulate the roles of Cdc20 in SAC signaling and mitotic exit remain unclear, particularly in situations where the checkpoint remains activated during an extended mitotic arrest. Although activation of the SAC should inhibit APC/C function indefinitely, a prolonged mitotic arrest results in the gradual and ongoing degradation of APC/C substrates, such as cyclin B1, ultimately allowing cells to escape this arrest and exit mitosis into a tetraploid G1 state. This phenomenon, known as “mitotic slippage”, allows cells to escape cell death induced by prolonged mitotic arrest. Prior work has found that mitotic slippage limits the efficacy of anti-mitotic drugs to kill cancer cells. However, mutations in checkpoint genes are rarely found in human cancer cells, suggesting that varying SAC behavior and mitotic timing may instead result from differences at the transcriptional, translational, or post-translational levels. Although the pathways that promote mitotic slippage remain incompletely understood, this phenomenon requires Cdc20-mediated activation of the APC/C. Thus, differences in Cdc20 expression and/or regulation could alter the efficacy of the spindle assembly checkpoint and the extent of mitotic slippage in cancer cells treated with anti-mitotic drugs.

Here, it is shown that human cells express alternative translational isoforms of Cdc20. The newly-identified N-terminally truncated Cdc20 isoforms are the products of translation initiation at downstream in-frame start codons. It is demonstrated that a truncated Cdc20 (43-499) isoform is functional in APC/C activation for mitotic progression, but is resistant to inhibition by the SAC, allowing this isoform to promote mitotic slippage even when cells are treated with anti-mitotic drugs. It is proposed that changes in the relative levels of at least two Cdc20 isoforms, full-length Cdc20 and Cdc20 (43-499), modulate SAC function and mitotic arrest duration. Overall, these findings reveal insights into the regulation of mitotic exit after a prolonged mitotic arrest.

### Results

### Full-length Cdc20 Protein Is Not Essential in Human Cells Due to the Presence Of Alternative Cdc20 Isoforms

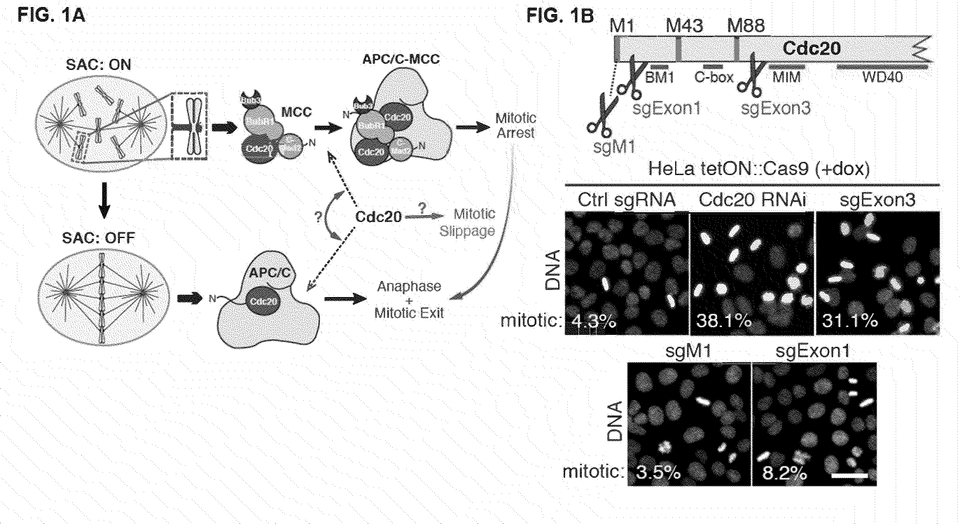

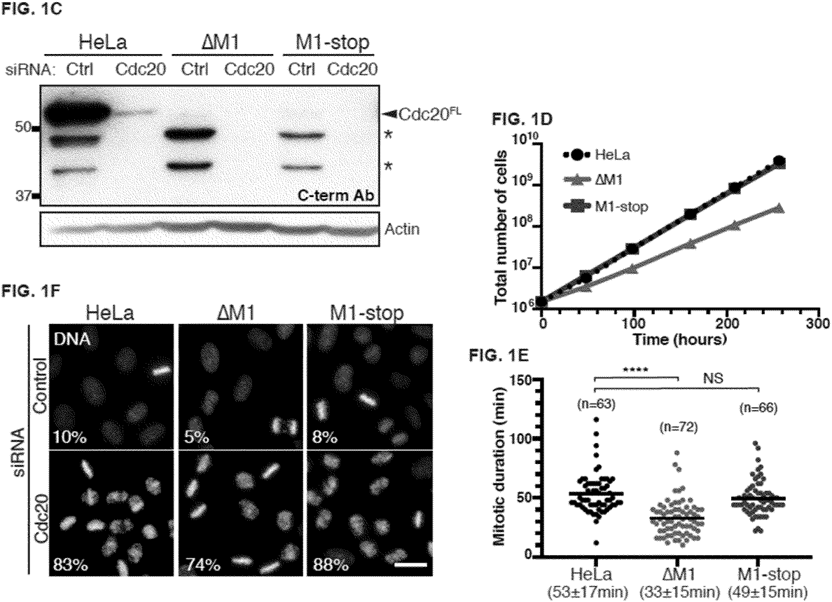

Cdc20 is an essential co-activator of the APC/C E3 Ubiquitin ligase that directs the degradation of mitotic substrates to drive chromosome segregation and mitotic exit. In the presence of improper kinetochore-microtubule attachments, cells activate the spindle assembly checkpoint (SAC), which targets Cdc20 and inhibits the APC/C to delay anaphase onset and mitotic exit (FIG. 1A). Once all kinetochores achieve proper microtubule attachment, the SAC is turned off and APC/C-Cdc20 promotes anaphase onset and mitotic exit. Cdc20 depletion results in a well-documented and potent mitotic arrest in metaphase due to the failure to activate the APC/C. Consistent with prior reports, a clear mitotic arrest in HeLa cells following treatment with Cdc20 siRNAs or using an inducible CRISPR/Cas9 gene-targeting strategy with an sgRNA recognizing a region within exon 3 (sgExon3) was observed (FIG. 1B). In contrast, Cas9-mediated cleavage with guides targeting the region near the start codon (sgM1) or exon 1 (sgExon1) of the CDC20 gene did not cause a potent mitotic arrest.

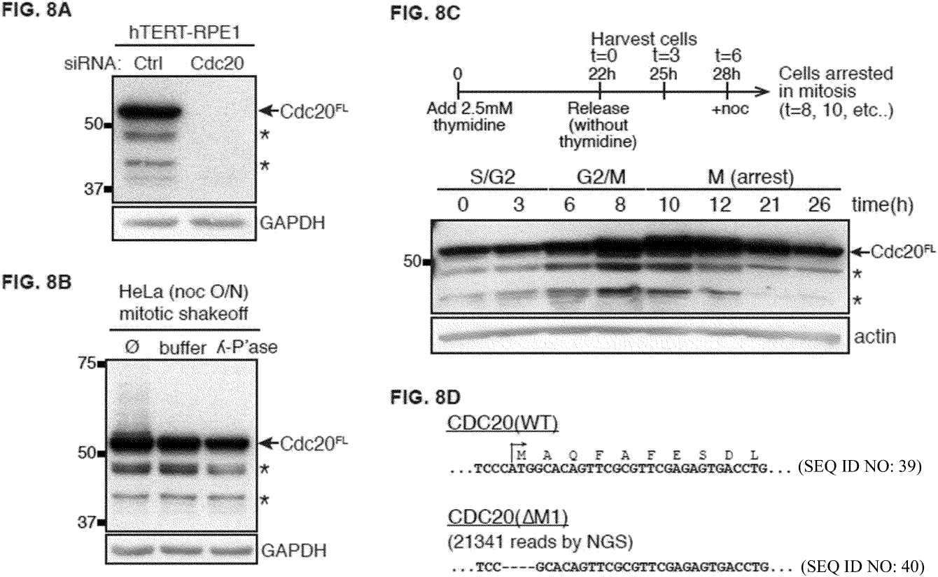

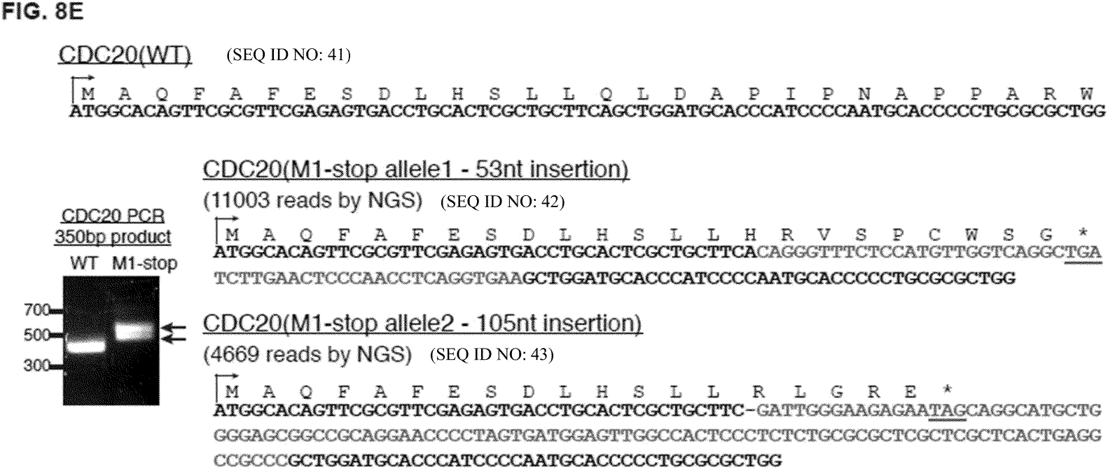

To define the basis for the differential effects of the Cdc20 gene disruptions, Cdc20 protein levels were analyzed by Western blotting using antibodies recognizing the C-terminus of human Cdc20 (aa 450-499). In addition to the presence of a protein matching the predicted molecular weight of full-length Cdc20 (55 kDa), the antibody detected two lower molecular-weight Cdc20 species. These protein bands were eliminated by Cdc20 siRNA treatment (FIG. 1C), indicating that they originate from the Cdc20 mRNA. Similar lower molecular-weight Cdc20 species were also detected in the non-transformed hTERT-immortalized retinal pigment epithelial cell line, hTERT RPE-1, suggesting that this phenomenon is not limited to transformed cancer cells (FIG. 8A). This altered migration in SDS-PAGE gels was not due to phosphorylation, as the smaller bands were not altered by lambda phosphatase treatment (FIG. 8B). These additional Cdc20 protein bands were present throughout the cell cycle, starting in S phase and persisting in cells undergoing a prolonged mitotic arrest induced by treatment with the microtubule-depolymerizing drug, nocodazole (FIG. 8C).

Although Cdc20 is essential for viability, Applicants were able to isolate stable clonal cell lines with the canonical full-length Cdc20 protein knocked out using CRISPR/Cas9 targeted with either the sgM1 or the sgExon1 guide (FIG. 1C). First, Applicants isolated a homozygous mutant cell line lacking the canonical M1 ATG start codon (ΔM1; see FIG. 8D for sequence information). Second, Applicants isolated a mutant (M1-stop) containing insertions of 53 nt and 105 nt respectively after the L14 residue (see FIG. 8E for sequence information) that result in premature stop codons in-frame with the M1 ATG start codon for both CDC20 alleles. For both the ΔM1 and M1-stop mutants, the lower molecular-weight Cdc20 protein bands detected by Western blotting were now the major Cdc20 species present and these were eliminated by Cdc20 siRNA treatment (FIG. 1C). This indicates that the lower77 molecular-weight forms present in the ΔM1 and M1-stop cells are not a result of degradation or cleavage of the full-length protein, but instead reflect N-terminally truncated alternative Cdc20 protein isoforms.

Despite the absence of the full-length Cdc20 protein, both the ΔM1 and M1-stop mutant cell lines were viable. The M1-stop mutant displayed a similar growth behavior (FIG. 1D) and mitotic duration to control cells (FIG. 1E; 49 min compared to 53 min for control HeLa cells). In contrast, the ΔM1 mutant displayed a modest growth defect (FIG. 1D) and progressed through mitosis significantly faster than control cells (33 min) (FIG. 1E), which may account for this growth defect. Importantly, the viability of both mutants lacking full-length Cdc20 was not a result of compensatory second-site suppressor mutations, as treatment with Cdc20 siRNAs resulted in a potent metaphase arrest similar to that observed in control HeLa cells (FIG. 1F). Thus, the ΔM1 and M1-stop mutant cell lines are still dependent on Cdc20 for mitotic progression. Together, these results demonstrate that human cells express multiple Cdc20 isoforms such that the canonical full-length Cdc20 protein is not strictly essential for mitotic progression or viability.

### Cdc20 Isoforms Are Produced by Alternative Translation Initiation at Downstream In-Frame Start Codons

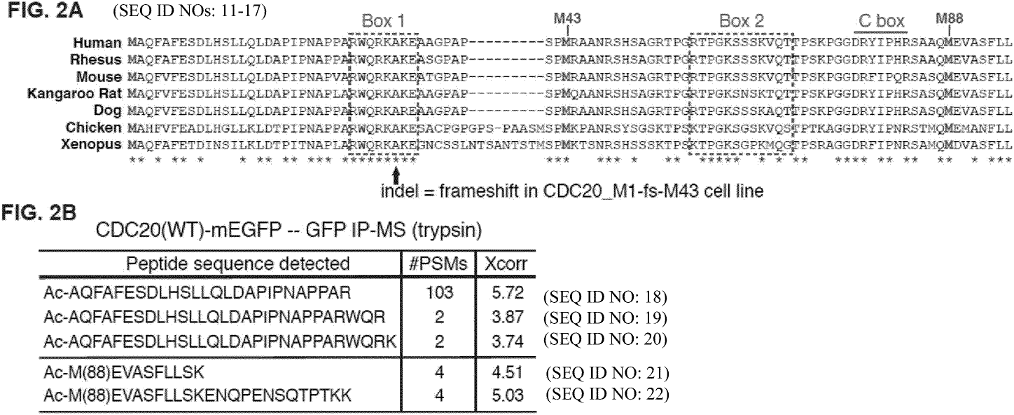

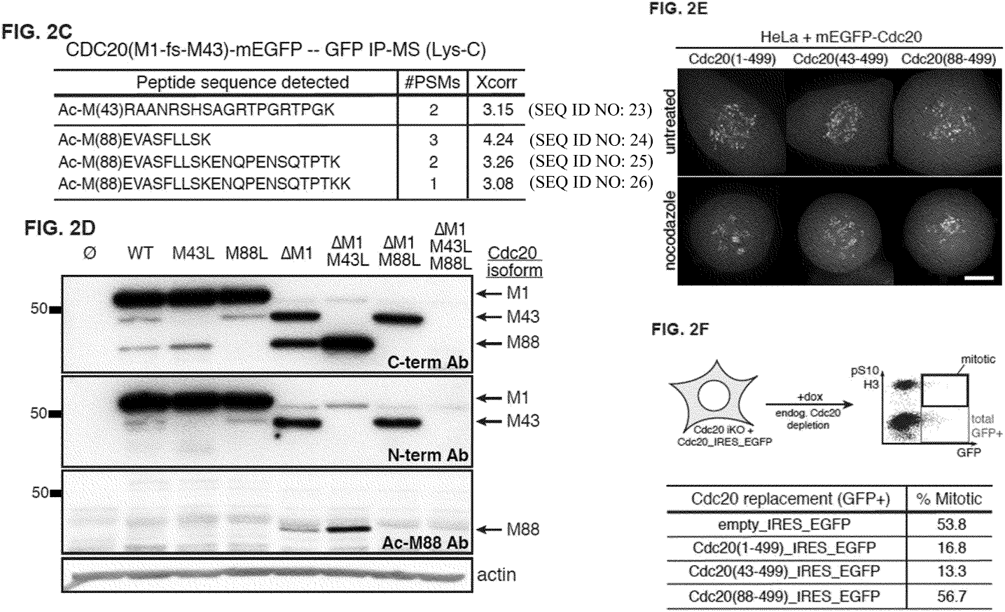

The nature of these alternative Cdc20 isoforms was next sought to be determined. By analyzing the Cdc20 protein sequence, two potential downstream translation start sites at positions 43 and 88 were identified that would produce protein products with predicted molecular weights (50 kDa and 45 kDa) that correspond to those of the species detected by Western blotting (FIG. 2A). These methionine 43 and 88 residues are conserved across mammals and diverse tetrapod species (FIG. 2A). As the Cdc20 isoforms detected by Western blotting share the same C-terminus based on the ability of the C-terminal antibody to recognize these products, the endogenous CDC20 gene locus in HeLa cells were tagged with a C-terminal GFP-tag. Cdc20-GFP protein was isolated by immunoprecipitation (IP) from mitotically enriched samples and determined the peptide sequences of all isolated Cdc20 isoforms by mass spectrometry (MS). In addition to N-terminally acetylated peptides corresponding to translation initiation at the annotated M1 start site, acetylated peptides indicative of translation initiation at M88 were also recovered (FIG. 2B). Likely due to the presence of neighboring arginine residues, tryptic peptides surrounding the M43 region were not recovered. Therefore, Applicants modified there IP-MS approach to use the endopeptidase LysC, which only cleaves after lysine residues. For these experiments, a cell line lacking the full-length Cdc20 protein (CDC20_M1-fs-M43; see below) to maximize identification of alternate isoforms was additionally used (FIG. 2A). In mitotically enriched samples, peptides with N-terminal acetylation corresponding to translation initiation at both the M43 and M88 start sites were identified (FIG. 2C). Thus, this mass spectrometry analyses suggest that the Cdc20 mRNA is subject to alternative translation initiation in human cells with both the M43 and M88 residues acting as alternative start codons (alternate translation start sites).

To assess alternative translation initiation at these downstream translation start sites, Cdc20 start-codon mutants were next tested. For these experiments, a replacement strategy combining untagged siRNA-resistant CDC20 cDNA constructs under the control of a doxycycline-inducible promoter with the depletion of endogenous Cdc20 protein by siRNA treatment were used. Following mitotic enrichment with nocodazole treatment, Western blotting was performed to identify the Cdc20 isoforms produced from an ectopic CDC20 cDNA (FIG. 2D). To avoid bias based on the specific antibody used to detect the protein isoforms, Applicants probed with 3 distinct Cdc20 antibodies: (1) the previously used polyclonal antibody recognizing the human Cdc20 C-terminus (aa 450-499), (2) a monoclonal antibody raised against the N-terminus of human Cdc20 (aa 1-175) whose epitope likely lies upstream of amino acid 88, and (3) a polyclonal antibody generated against the acetylated M88-terminus identified by mass spectrometry. Using this gene-replacement strategy, it was found that the wild-type CDC20 cDNA construct recapitulated the Cdc20 isoform pattern observed in control HeLa cells with the presence of 3 isoforms, but with reduced levels of the truncated isoforms compared to the full-length protein. The behavior of this wild-type Cdc20 cDNA replacement construct suggests that the Cdc20 isoforms are generated from a single transcript, rather than by alternative mRNA splicing. Importantly, mutating M43 or M88 to leucine in this cDNA construct selectively eliminated the corresponding protein products, indicating that these start codons are responsible for the production of the truncated Cdc20 isoforms. As expected, deletion of the M1 start codon abrogated expression of the full-length Cdc20 protein. However, the ΔM1 mutant also resulted in increased translation initiation at the downstream start codons and thus increased levels of both truncated isoforms, similar to the ΔM1 mutant cell line (FIG. 1C). Mutating M43 or M88 to leucine in the ΔM1 construct again eliminated the expression of the M43 or M88 isoform respectively, indicating that these truncated Cdc20 isoforms indeed originate from alternative translation initiation at the respective downstream start codon.

To test the functional properties of the alternative Cdc20 proteins, their cellular localization and ability to promote mitotic progression were next analyzed. Similar to the full-length Cdc20 protein, the M43 and M88 isoforms localized to kinetochores in both untreated or nocodazole-treated HeLa cells when expressed as N-terminal mEGFP-Cdc20 fusions (FIG. 2E). This indicates that the loss of N-terminal Cdc20 residues (up to 87 aa) does not abrogate its kinetochore recruitment and is consistent with prior findings that Cdc20 is recruited to kinetochores via motifs in the downstream regions of the protein. To test the ability of the M43 and M88 isoforms to support mitotic progression, FACS analysis was used to quantify cells in mitosis. In cells depleted of all isoforms of endogenous Cdc20 protein (using CRISPR/Cas9 with the sgExon3 guide RNA), a high percentage of mitotic cells indicative of a potent mitotic arrest was observed. This mitotic arrest phenotype was suppressed by expression of guide RNA-resistant versions of either full-length Cdc20 (1-499) or Cdc20 (43-499), but not Cdc20 (88-499) (FIG. 2F). Thus, although both the M43 and M88 isoforms localize to kinetochores, only the M43 isoform is able to complement the loss of endogenous Cdc20 protein in promoting mitotic progression to an extent comparable to that of full-length Cdc20. This inability of the M88 isoform to effectively promote mitotic progression is likely due to the absence of the critical “C-box” motif at residues 77-83, which is required for binding to the APC/C (FIG. 2A). Together, these results demonstrate that human cells express alternative Cdc20 translational isoforms and suggest that the M43 isoform can confer mitotic progression and viability in cells lacking full-length Cdc20.

### Truncated Cdc20 Isoforms Are Inefficient Targets of the SAC and Promote Mitotic Slippage

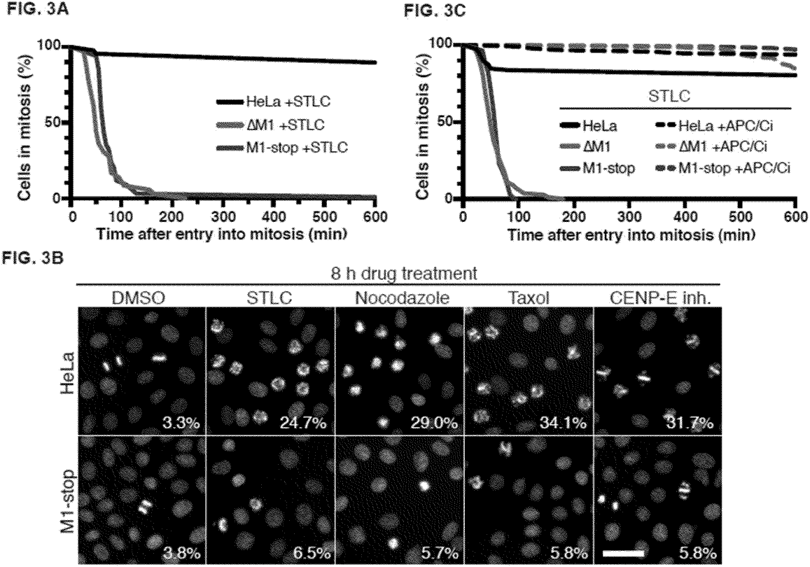

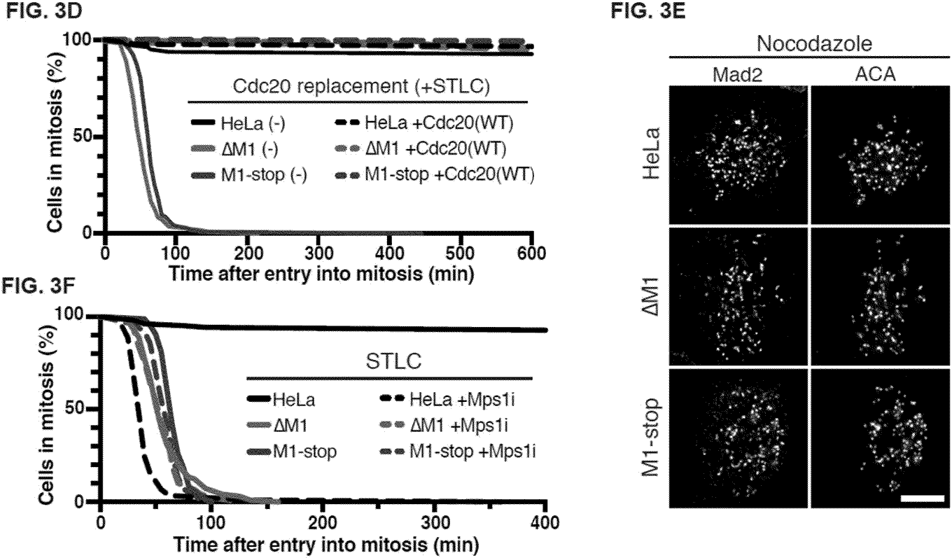

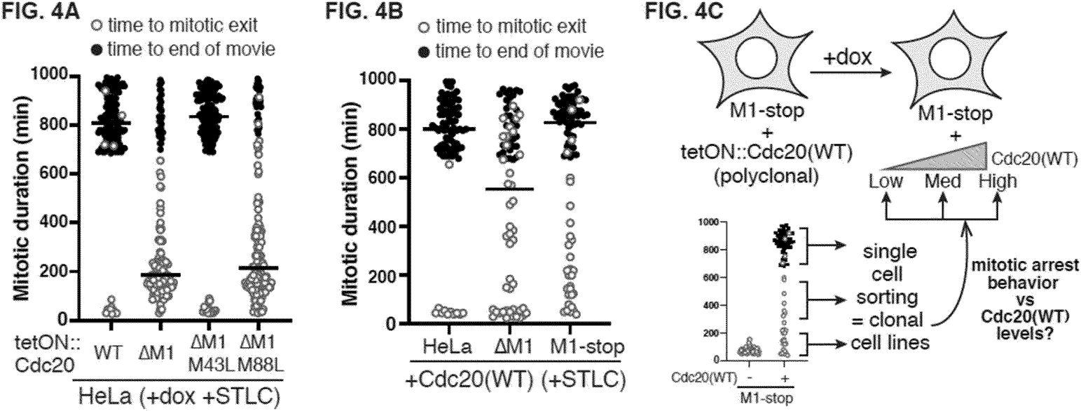

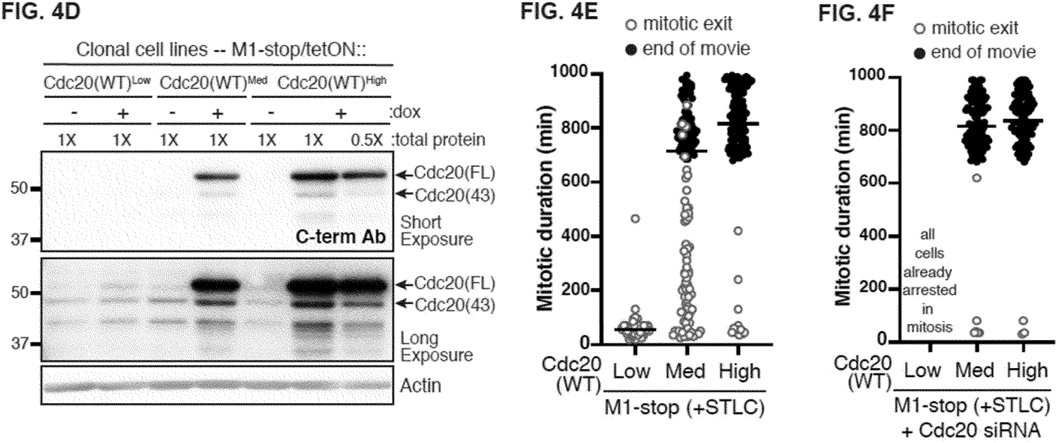

Although eliminating the canonical full-length Cdc20 protein in the ΔM1 and M1-stop mutants did not compromise cellular viability or result in a cell cycle arrest in unperturbed cells due to the presence of the alternative M43 isoform, Applicants next considered whether these mutants may alter the ability of cells to respond to spindle assembly checkpoint signaling. Both the M43 and M88 isoforms lack a conserved motif (Box1 or BM1; aa 27-34) that is required for robust Cdc20-Mad2 interactions and SAC signaling (FIG. 2A). Treatment with the Eg5/Kif11-inhibitor STLC prevents bipolar spindle formation, resulting in potent SAC activation and an extended mitotic arrest. In time-lapse experiments, after entering mitosis, STLC-treated control HeLa cells remained arrested in mitosis for the duration of the analysis (>10 h) (FIG. 3A). In contrast, both the ΔM1 and M1-stop mutant cell lines displayed potent SAC defects as they were able to exit mitosis within a few hours despite the presence of STLC (FIG. 3A). A similar failure to arrest in mitosis relative to control HeLa cells was also observed when M1-stop cells were treated with diverse anti-mitotic drugs that activate the SAC (FIG. 3B).

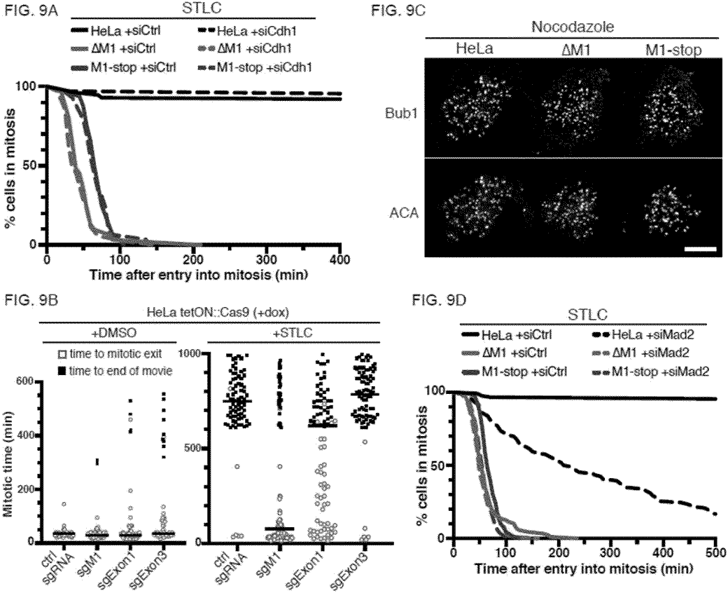

To assess whether the mitotic slippage behavior of the mutant cell lines is due to premature APC/C activation, Applicants treated cells with both STLC and the APC/C-inhibitor proTAME. APC/C inhibition suppressed the premature mitotic exit observed in ΔM1 and M1-stop cells, resulting in a prolonged mitotic arrest (FIG. 3C). In addition to Cdc20, Cdh1 acts as a co-activator of the APC/C in late mitosis. Applicants considered the possibility that, upon loss of full-length Cdc20, Cdh1 could substitute for Cdc20 to promote premature APC/C activation. However, Cdh1 depletion using siRNA treatment did not alter the premature mitotic exit of the ΔM1 and M1-stop mutant cell lines (FIG. 9A). Finally, Applicants found that Cdc20 replacement with ectopic wild-type Cdc20 cDNA restored the prolonged mitotic arrest behavior to both the ΔM1 and M1-stop cell lines (FIG. 3D). Therefore, the SAC defects in these mutant cell lines are due to the absence of full-length Cdc20 protein rather than other potential second-site mutations. In addition, similar SAC defects were observed upon acute depletion of full-length Cdc20 by Cas9 induction in cell lines expressing either the sgM1 or sgExon1 guide RNAs (FIG. 9B) indicating that these behaviors are not a result of long-term adaptation in the stable mutant cell lines. Together, these results demonstrate that the loss of full-length Cdc20 impairs SAC function and results in an APC/C-and Cdc20-dependent mitotic exit in the presence of anti-mitotic drugs.

The premature mitotic exit observed in the M1-stop and ΔM1 mutant cell lines could reflect a defect in the upstream SAC signaling pathway. To test whether SAC activation occurs in these mutants, the localization of the SAC proteins, Mad2 and Bub1, which are recruited to unattached kinetochores to trigger checkpoint signaling, were tested. Mad2 and Bub1 localized to kinetochores in ΔM1 and M1-stop mutant cell lines treated with the microtubule-depolymerizing drug nocodazole similar to control HeLa cells (FIGS. 3E, 9C). However, despite evidence for upstream SAC activation, both the ΔM1 and M1-stop mutant cell lines behaved functionally as if the SAC was defective (FIG. 3A). Indeed, the premature mitotic exit observed for the ΔM1 mutant in the presence of STLC was not exacerbated further by weakening the SAC by treatment with either the Mps1 inhibitor, AZ3146, which targets the most upstream component of the SAC signaling cascade (FIG. 3F, Table S1), or using siRNAs against Mad2, a key component of the SAC effector complex (FIG. 9D, Table S1). The M1-stop mutant also displayed a significantly reduced mitotic arrest duration compared to control HeLa cells. However, targeted SAC inhibition using AZ3146 or Mad2 siRNAs was able to further reduce the arrest of the M1-stop mutant, resulting in a mitotic duration similar to that of the ΔM1 mutant (Table S1). Thus, both the ΔM1 and M1-stop mutant cell lines are viable, but display a significant ability to bypass the mitotic arrest induced by anti-mitotic drugs. Together, our results suggest that, even under conditions when the SAC is active, the truncated Cdc20 isoforms in the ΔM1 and M1-stop cell lines are not effectively targeted and inhibited by the SAC, resulting in premature APC/C activation and mitotic slippage.

Table S1. The SAC is defective in ΔM1 and M1-stop mutant cell lines.

Mitotic arrest duration in the presence of 10 µM STLC for control HeLa compared to the ΔM1 and M1-stop mutant cell lines with the indicated drug or RNAi treatment (see FIGS. 3F, 9D). Indicated is the mean mitotic duration ± standard deviation across two experimental replicates. Statistics from Mann-Whitney Test (**** = p < 0.0001, NS = not significant).

Mitotic time (min)

| | HeLa | ΔM1 | M1-stop |
|---|---|---|---|
| STLC/DMSO | 780±203 (n=103) | 60±24 (n=97) | 67±12 (n=85) |
| STLC/MPS1i (4 µM) | 43±37 (n=89) | 56±19 (n=79) | 60±13 (n=98) |
| | <0.0001 (^^^^) | NS | <0.001 (⁺⁺⁺) |
| STLC/siCtrl | 805±168 (n=122) | 65±37 (n=88) | 70±18 (n=90) |
| STLC/siMad2 (24 hr) | 284±225 (n=106) | 57±25 (n=99) | 59±20 (n=97) |
| | <0.0001 (****) | NS | <0.0001 (****) |

### The Relative Levels of Cdc20 Translational Isoforms Influence Mitotic Arrest Duration

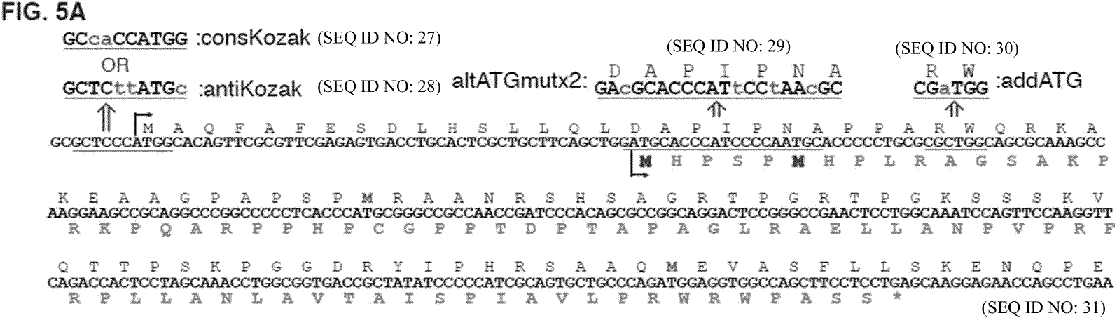

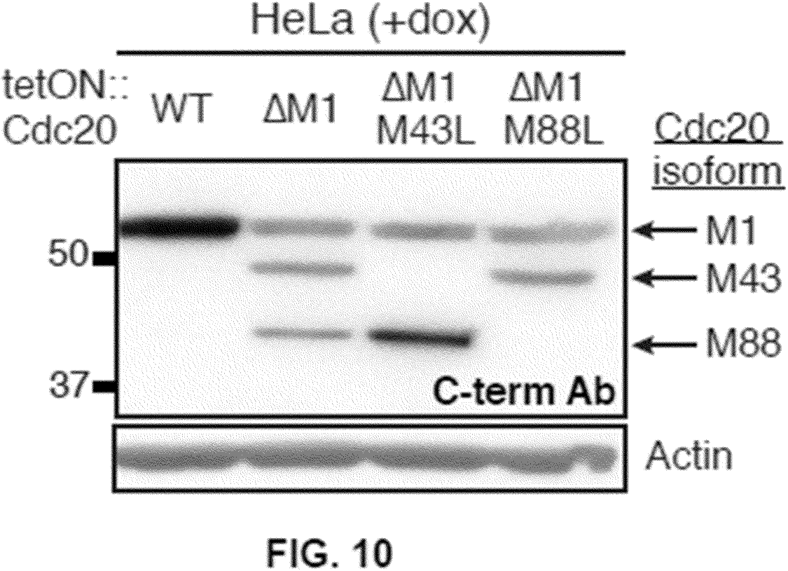

Our analysis of the ΔM1 and M1-stop mutant cell lines indicates that, in the absence of full-length Cdc20, the alternative Cdc20 isoforms are checkpoint-defective such that they prematurely activate the APC/C and promote mitotic slippage in the presence of anti-mitotic drugs. However, cells normally express a balance of multiple Cdc20 translational isoforms, including the full-length protein (FIG. 1C; FIG. 8A). Thus, Applicants next sought to determine whether altered levels of the truncated Cdc20 isoforms are sufficient to promote mitotic slippage in the presence of the full-length protein. For these experiments, Applicants used ectopic expression of a CDC20-ΔM1 cDNA construct, which results in increased levels of the shorter Cdc20 isoforms relative to full-length Cdc20 (FIG. 10). Ectopic expression of wild-type CDC20 cDNA did not substantially alter mitotic arrest duration in the presence of STLC (FIG. 4A). In contrast, CDC20-ΔM1 expression caused a dramatic increase in mitotic slippage even in the presence of endogenous full-length Cdc20 (FIG. 4A). Preventing increased expression of the M43 isoform in the context of the ΔM1 construct using an M43L mutation fully suppressed the induced mitotic slippage phenotype, whereas the M88L mutation had little effect. These results show that increased levels of the Cdc20 (43-499) isoform are sufficient to promote mitotic slippage even in HeLa cells expressing full-length Cdc20 protein.

As an alternative strategy to modulate the relative levels of the Cdc20 translational isoforms, the ΔM1 and M1-stop mutant cell lines were utilized, which lack full-length Cdc20 and display a short mitotic arrest duration upon STLC treatment. Ectopic expression of the wild-type CDC20 cDNA from an integrated construct under the control of a doxycycline-inducible promoter restored full-length Cdc20 protein in the mutant cells and suppressed the premature mitotic exit phenotype in the presence of STLC (FIG. 4B). However, these polyclonal cell lines displayed heterogenous mitotic arrest behaviors so Applicants decided to generate and analyze clonal cell lines of the M1-stop mutant expressing the doxycycline-inducible CDC20 construct (FIG. 4C). By analyzing multiple clones, Applicants identified a correlation between the mitotic arrest behavior of a given clone and the expression level of the integrated doxycycline-inducible CDC20 construct (FIGS. 4D-E). Clones with negligible (low) expression of full-length Cdc20 did not rescue the mitotic slippage phenotype and displayed a short mitotic arrest duration. In contrast, for clones with high expression from the wild-type CDC20 construct, nearly all cells remained arrested in mitosis for the duration of the analysis. Interestingly, clones expressing an intermediate (medium) level of full-length Cdc20 protein from the integrated construct displayed heterogeneous mitotic arrest behavior. At this intermediate level of full-length Cdc20 protein, stochastic differences in Cdc20 expression in individual cells in the population may lead to variations in Cdc20 isoform ratio that impact their mitotic arrest timing and behavior. To determine whether this stochastic behavior was due to the relative levels of Cdc20 isoforms, Applicants used RNAi to deplete the endogenous truncated isoforms present in the M1-stop mutant. Treatment with Cdc20 siRNAs completely suppressed the heterogeneous mitotic arrest behavior and abrogated the observed mitotic slippage (FIG. 4F), but did not affect cell cycle progression in the absence of STLC (data not shown).

Overall, these results are consistent with a model in which the relative levels of Cdc20 isoforms influence the mitotic arrest behavior of individual cells. Together, this suggests that changes in the levels of Cdc20 isoforms could modulate mitotic arrest duration resulting in mitotic slippage.

### Translation Initiation at Alternative Out-of-frame Start Codons Modulates Cdc20 Isoform Expression Levels

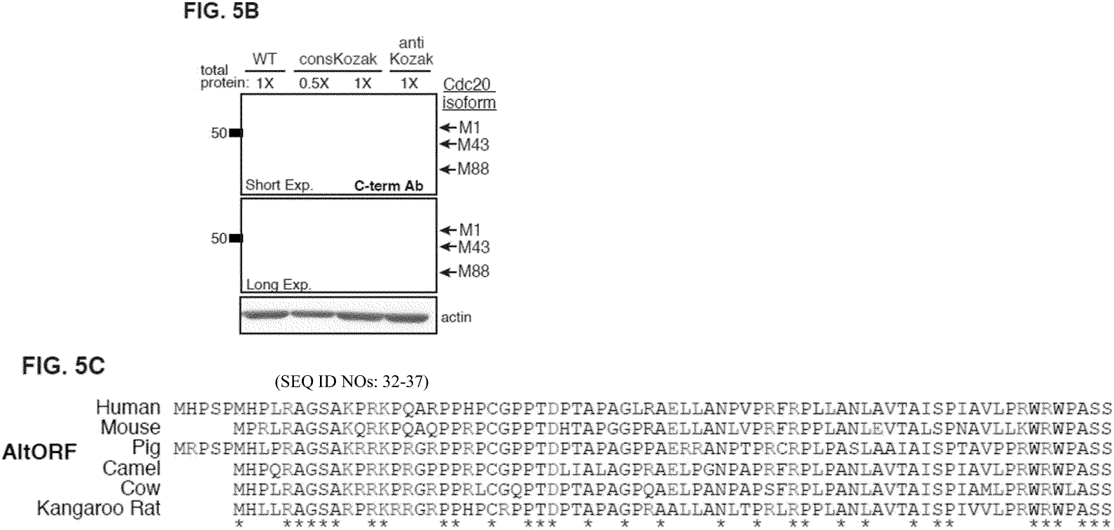

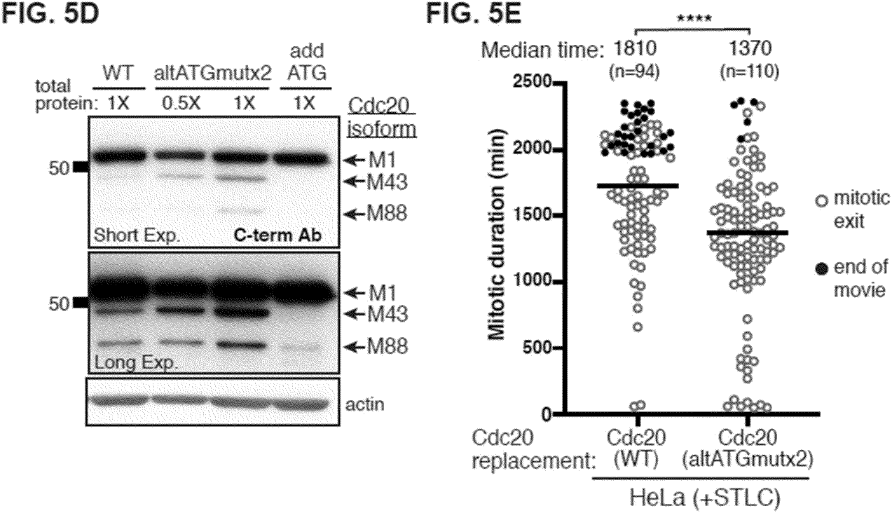

Given the observation that the relative levels of the different Cdc20 translational isoforms impact mitotic arrest duration, Applicants next investigated the molecular mechanisms that underlie the control of Cdc20 translational initiation and start site selection. Eukaryotic translation initiation is generally accomplished by a scanning mechanism in which the 40S ribosomal subunit is loaded at the 5′-cap and translocates along the mRNA until it initiates translation at the first AUG encountered. However, translation at downstream start codons can occur by either translational re-initiation or leaky scanning in which a fraction of 40S ribosomal subunits continue scanning beyond the first AUG to initiate at a downstream AUG. Consistent with leaky ribosome scanning, it was observed that deletion of the M1 start codon in either the ΔM1 mutant cell line (FIG. 1C) or the ΔM1 ectopic construct (FIG. 2D) resulted in increased accumulation of Cdc20 protein that initiated at the downstream M43 and M88 start codons. Leaky ribosomal scanning occurs when the translational context of the first AUG is suboptimal. Using a replacement strategy with siRNA-mediated depletion of the endogenous Cdc20 protein, it was assessed whether changes in the translation-initiation context of M1 affect the expression of the truncated M43 and M88 Cdc20 isoforms (FIG. 5A). Introducing the strong consensus Kozak sequence at the M1 start site would increase translation initiation at this start codon and thus reduce leaky scanning and downstream translation initiation. Indeed, for this “consKozak” construct, Applicants observed increased levels of the full-length Cdc20 protein compared to the wild-type construct and a concomitant decrease in translation of the M43 isoform, although the mutation only partially affected the M88 isoform (FIG. 5B). Conversely, further weakening the translational context at the M1 start site with an anti-Kozak sequence reduced expression of the full-length protein while allowing increased translation initiation at the M43 and M88 start sites. These results are therefore consistent with translation initiation at the M43 and M88 start sites relying on leaky ribosome scanning and suggest that the translational context surrounding the M1 start site determines expression of the M43 isoform.

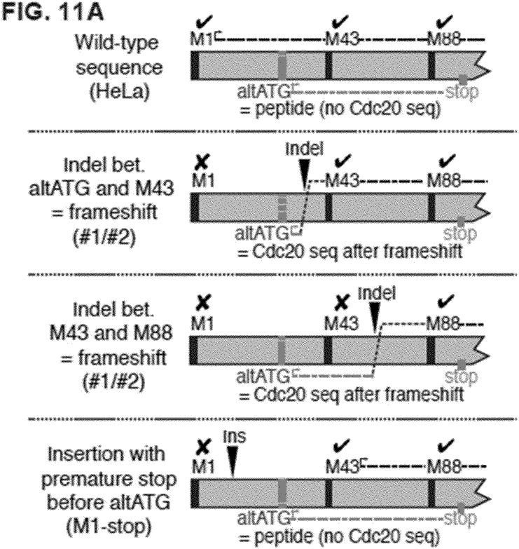

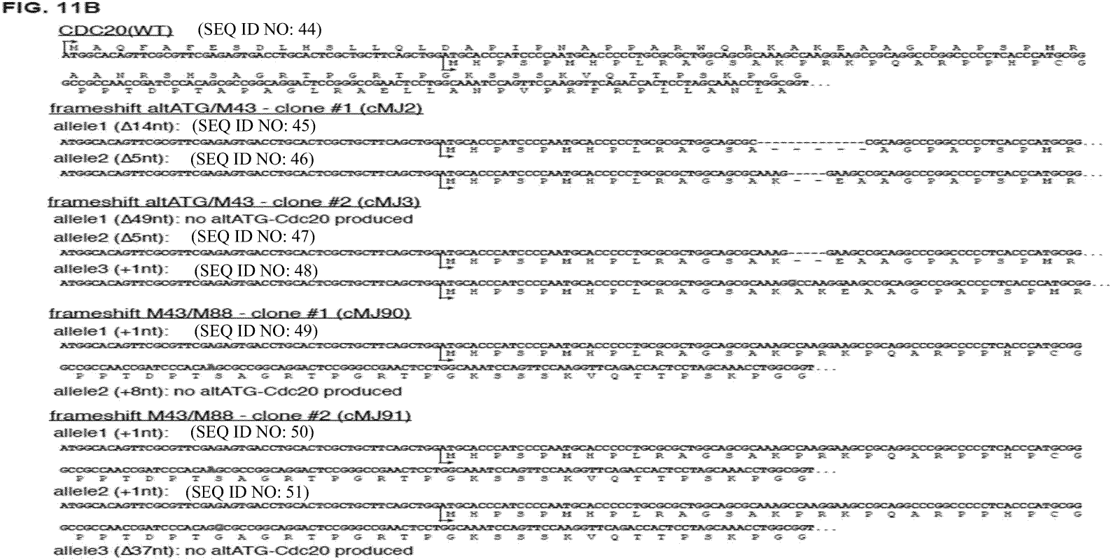

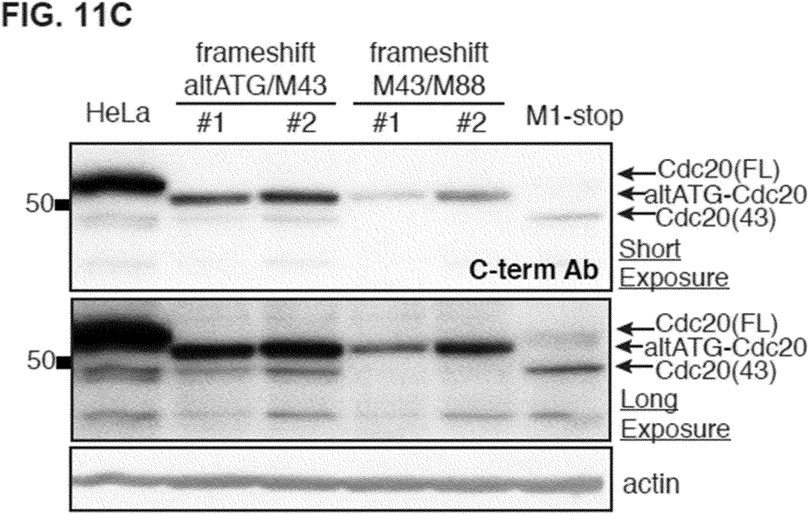

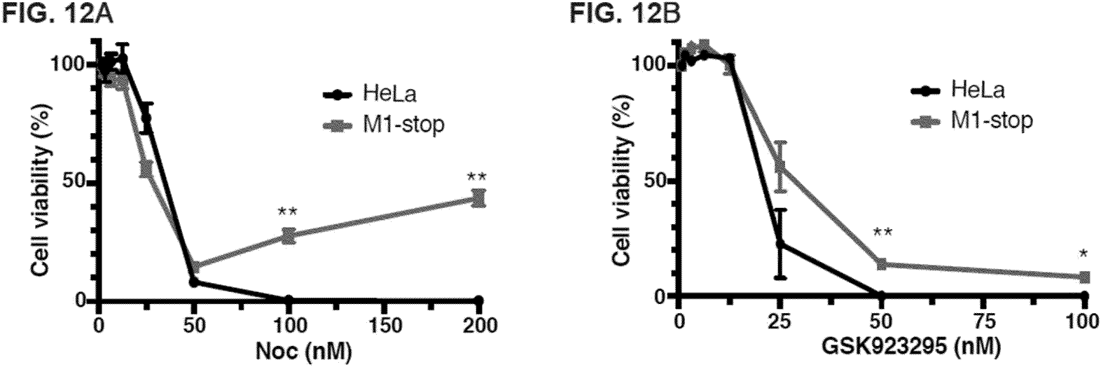

However, analysis of the CDC20 mRNA sequence revealed that M43 and M88 are not the only potential start codons downstream of the annotated M1 start site. In fact, two alternative out-of-frame start codons between M1 and M43 were identified (FIG. 5A), which are predicted to capture scanning ribosomes, thus preventing a subset of 40S ribosomal subunits from reaching M43 or M88. The presence of these out-of-frame start codons is conserved across mammals with a conserved stop site present downstream of the M88 codon in each case (FIG. 5C). Translation of this alternative open reading frame (altORF) would therefore be expected to prevent initiation at either the M43 or M88 start codon. Applicants first investigated whether translation initiation occurs at these alternative out-of-frame start codons resulting in an altORF peptide. To test this, clonal cell lines with indel mutations after the alternative start codons were generated at the endogenous locus in all CDC20 alleles, thereby disrupting translation of the full-length Cdc20 protein. Applicants focused on clones in which the mutation of at least one CDC20 allele resulted in a frame shift that would be predicted to connect the altORF peptide with amino acid sequences encoding downstream regions of Cdc20 (FIG. 11A, see FIG. 11B for sequence information). If the altORF is translated, this would produce a chimeric protein (altATG-Cdc20) that is shorter than full-length Cdc20, but is detectable with antibodies against the Cdc20 C-terminus. Western blot analysis of mitotically-enriched cells showed that all mutant clones tested contained a new Cdc20 band that migrated at a similar molecular weight independent of the specific indel mutation present within the CDC20 gene (FIG. 11C), suggesting that they initiated at the same alternative translation start site. In contrast, insertions upstream of the alternative out-of-frame start codons, such as those in the M1-stop mutant, only abrogated expression of the full-length Cdc20 protein, without generating a chimeric altATG-Cdc20 protein. These observations indicate that the alternative out-of-frame AUGs within CDC20 are functional for translation initiation.

The presence of the alternative start sites within CDC20 is predicted to negatively impact translation initiation at the downstream M43 and M88 sites. In this case, mutating the altORF start codons using silent mutations that do not otherwise disrupt the Cdc20 coding sequence should increase expression of the truncated Cdc20 isoforms (FIG. 5A). To test this, Applicants assessed ectopic Cdc20 isoform expression using a replacement strategy with siRNA-mediated depletion of the endogenous protein. Indeed, mutating the alternative start sites (altATGmutx2) resulted in increased levels of the M43 and M88 isoforms compared to the wild-type control without altering the levels of the full-length protein (FIG. 5D). Alternatively, introducing additional out-of-frame start codon(s) before the M43 start site should capture more scanning ribosomes and further reduce translation initiation at the downstream M43 and M88 sites. Applicants therefore introduced a single nucleotide substitution in our cDNA construct to create a new start codon at a position with a strong predicted translation-initiation context but without disrupting the Cdc20 coding sequence. For this “addATG” construct, it was observed that translation of the M43 isoform was completely blocked, although the mutation only partially affected the M88 isoform (FIG. 5D). These results are consistent with translation initiation at the M43 start site relying on leaky ribosome scanning. Alternative start sites downstream of M1 therefore capture scanning ribosomes to modulate the expression of the truncated Cdc20 isoforms.

To assess the contribution of these alternative out-of-frame start codons on the mitotic arrest behavior of HeLa cells, Applicants evaluated the consequences of disrupting the altORF. Applicants performed replacement assays by depleting endogenous Cdc20 with siRNAs to compare a wild-type siRNA-resistant CDC20 construct to the altATGmutx2 mutant. Cells expressing the wild-type CDC20 construct displayed modest mitotic slippage upon STLC treatment, but a high proportion of cells remained arrested in mitosis for >2000 min or until the end of our time lapse analysis (FIG. 5E). In contrast, replacement with the altATGmutx2 construct, which increases the relative levels of the truncated Cdc20 isoforms, resulted in an increase in mitotic slippage and a significant reduction in the mitotic arrest duration, from a median time of 1810 min for the wild-type CDC20 construct to 1370 min for the altATGmutx2 mutant. Therefore, translation initiation at the alternative out-of-frame start sites dampens expression of the truncated Cdc20 isoforms to extend mitotic arrest. Increasing the level of these truncated Cdc20 isoforms by disrupting altORF expression had little impact on the levels of the full-length protein (FIG. 5D), but decreased the mitotic arrest duration of HeLa cells (FIG. 5E), consistent with a model in which the relative levels of Cdc20 isoforms modulate mitotic arrest behavior.

Overall, this work suggests that expression of the M43 and M88 isoforms relies on leaky ribosome scanning downstream of the M1 start site. The presence of alternative translation sites before the M43 start codon provides a conserved mechanism to modulate translation initiation at the downstream in-frame start sites.

### Downstream In-frame Cdc20 Translation Initiation Influences the Mitotic Slippage Behavior of Cancer Cell Lines

To test the requirements for mitotic slippage across cancer cell lines, Applicants analyzed the mitotic arrest behavior in HeLa cells, the osteosarcoma U2OS cell line, and the lung adenocarcinoma A549 cell line. Although all three cell lines displayed mitotic slippage upon treatment with STLC, Applicants observed widely varying mitotic arrest durations between cell lines with median mitotic arrest times of 1645 min, 805 min, and 780 min for STLC-treated HeLa, U2OS, and A549 respectively, consistent with prior reports. In each case, Cdc20 depletion using siRNA treatment suppressed mitotic slippage and resulted in a sustained mitotic arrest and/or increased cell death, suggesting that all three cell lines undergo mitotic slippage due to premature Cdc20-mediated activation of the APC/C.

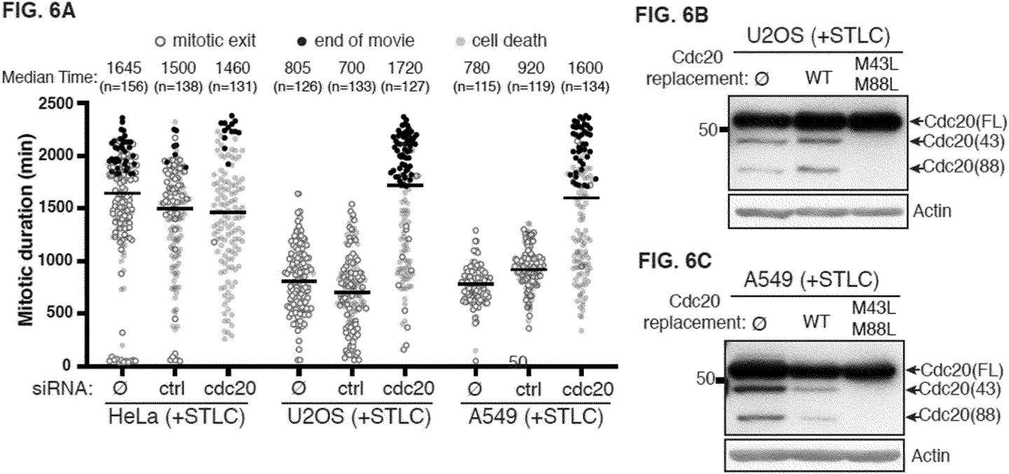

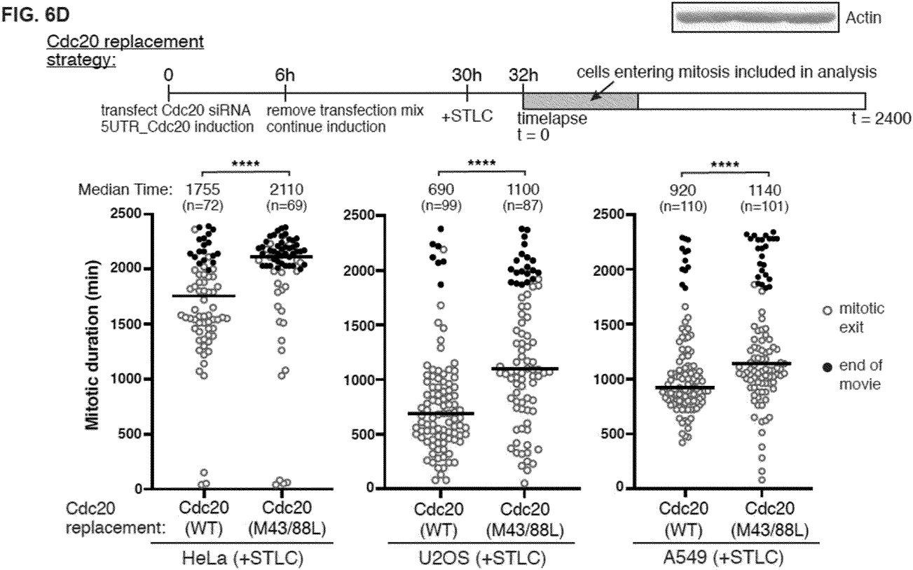

This work suggests a model in which the expression of truncated Cdc20 isoforms promotes mitotic slippage. Similar to work in HeLa cells, it was found that mitotically enriched U2OS and A549 cells also express the truncated Cdc20 isoforms (FIGS. 6B-C). To test the requirements for these truncated isoforms in promoting mitotic slippage, Applicants performed replacement assays in HeLa, U2OS, or A549 cells expressing siRNA-resistant 5′ UTR-CDC20 cDNA constructs from a doxycycline-inducible promoter combined with depletion of endogenous Cdc20 protein by siRNA treatment. Replacements with a wild-type CDC20 construct recapitulated the expected mitotic slippage behavior of each cell line, with median arrest times of 1755 min, 690 min, and 920 min for HeLa, U2OS, and A549 respectively (FIG. 6D). Applicants then generated replacements with a CDC20 construct in which the start codons of both M43 and M88 isoforms were mutated to leucine (Cdc20 M43L M88L) to prevent expression of the truncated Cdc20 isoforms. Strikingly, replacement of endogenous Cdc20 with Cdc20 M43L M88L resulted in a significant increase in the mitotic arrest duration for all three cell lines, with the median arrest times increasing to 2110 min, 1100 min, and 1140 min for HeLa, U2OS, and A549, respectively. These results are consistent with a model that truncated Cdc20 isoforms contribute to the mitotic slippage phenotype of these diverse cancer cell lines.

Although changes in the translational decoding of CDC20 mRNA could contribute to the varying degrees of mitotic slippage observed across different cancer cell lines, Applicants also considered whether genetic changes present within the CDC20 gene in tumor and cancer cell lines could impact the relative Cdc20 isoform levels. Through a survey of tumors and cancer cell lines using public databases, Applicants identified multiple distinct genetic mutations within CDC20 that are predicted to eliminate the full-length Cdc20 protein, thus increasing the relative levels of the M43 isoform (FIG. 7A). These mutations would be functionally equivalent to the mutations in the ΔM1 and M1-stop mutant cell lines described earlier and their impact on mitotic slippage behavior may have important consequences on the chemotherapeutic treatment of these cancers. In fact, Applicants observed that after 72 h treatment with a range of anti-mitotic drugs, the clonal M1-stop mutant cell line displayed increased cell viability compared to control HeLa cells at the highest drug concentrations (FIGS. 7B-C, 12A-B).

Together, these results indicate that the presence of truncated Cdc20 isoforms influence the mitotic slippage behavior of various cancer cell lines. Since the relative levels of the different Cdc20 translational isoforms can modulate mitotic arrest timing, Applicants propose that the differences in mitotic arrest behaviors observed across cell lines may also be influenced by the relative Cdc20 isoform levels present in those cells. Importantly, translational differences or genetic mutations that influence the relative Cdc20 isoform levels may affect cancer and tumor behavior and impact anti-mitotic drug sensitivity.

### Discussion

Anti-mitotic drugs block cell division and activate the spindle assembly checkpoint to elicit a prolonged mitotic arrest that ultimately leads to cell death. Unfortunately, some cancer cells can evade this arrest by “slipping” out of mitosis into interphase. Despite extensive work and an increasing understanding of the spindle assembly checkpoint, the factors that promote mitotic slippage in normal and cancerous cells have remained mysterious. Here Applicants investigated the role of Cdc20 in human cells and uncovered a critical link between the relative levels of Cdc20 translational isoforms and mitotic slippage. Together, this work supports a model in which the presence of alternative Cdc20 isoforms that are resistant to SAC-mediated inhibition are able to promote mitotic exit during a prolonged mitotic arrest even in the presence of mitotic perturbations.

An active spindle assembly checkpoint inhibits APC/C function to arrest cells in mitosis. This cellular response relies on the sequestration of Cdc20 protein within the MCC and the formation of inhibited APC/C-MCC-bound complexes to maintain a mitotic arrest (FIG. 7D). However, over the course of a prolonged mitotic arrest, mitotic APC/C substrates such as cyclin B1 are subject to gradual yet ongoing degradation. When cyclin B1 levels fall below the threshold required to maintain the mitotic state, cells undergo mitotic slippage and exit mitosis. Although this behavior requires Cdc20-mediated activation of the APC/C, it has been unclear how Cdc20 escapes SAC-mediated inhibition in the presence of continued SAC signaling. This work demonstrates that human cells express alternative Cdc20 translational isoforms such that the canonical full-length Cdc20 protein is not strictly essential for viability or mitotic progression. The alternative Cdc20 isoforms are N-terminally truncated proteins that originate from alternative translation initiation at downstream in-frame start codons. The M43 isoform can support mitotic progression in mutant cell lines lacking full-length Cdc20 protein and, more importantly, this Cdc20 isoform enables cells to escape from a prolonged mitotic arrest.

The Cdc20 N-terminal region contains a conserved motif (Box1 or BM1; aa 27-34) that is required for robust Cdc20-Mad2 interactions and subsequent incorporation of Cdc20 into the MCC. The M43 isoform lacks this critical motif and is therefore resistant to SAC-mediated inhibition. When the M43 isoform is the only Cdc20 isoform present in cells, MCC formation is likely disrupted, thus resulting in a SAC defect and premature mitotic exit (FIG. 7E). Although such mutant cells lines display accelerated mitotic progression compared to control cells, they are still viable. This indicates that a robust SAC response is not strictly required for cell viability under conditions where proper mitotic progression is not compromised. However, cells express multiple Cdc20 isoforms, including the full-length Cdc20 protein, which is efficiently incorporated in the MCC when the SAC is active (FIG. 7F). The combination of MCC formation and the presence of multiple Cdc20 molecules in the inhibited APC/C-MCC-bound complex means that APC/C complexes containing the M43 isoform could still be subject to SAC-mediated inhibition when bound to the MCC. The efficiency of APC/C-MCC interactions and APC/C inhibition may vary depending on which Cdc20 isoforms are present within the APC/C. In addition, the full-length and M43 isoforms may differ in their binding affinities with the APC/C. In those cases, competition between Cdc20 isoforms likely determines SAC effectiveness and mitotic arrest duration. Indeed, we observed that altering the relative levels of Cdc20 isoforms - in particular full-length Cdc20 and the truncated M43 isoform - influences the mitotic arrest behavior of individual cells. Future biochemical and structural studies analyzing the impact of the SAC-resistant M43 Cdc20 isoform and its interplay with the full-length protein will offer a more complete molecular picture of the function and regulation of Cdc20 during mitosis.

Analyses of cell fates following a prolonged mitotic arrest reveal extensive cell-to-cell variability both between cell lines and within the same cell line. Patient responses to anti-microtubule drugs also vary greatly, with some patients responding well to treatment, but others showing no improvement due to drug resistance. Mitotic slippage has important consequences for drug sensitivity, as cells that are more prone to mitotic slippage display increased cell viability upon treatment with anti-mitotic drugs (also see FIGS. 7B-C and 12A-B). Defining the molecular determinants that underlie these differences in cell fates and patient responses is critical to enable effective choices for cancer therapy in a patient-specific manner. The M43 isoform is resistant to SAC-mediated inhibition and appears to impart a basal level of APC/C activation that gradually degrades mitotic substrates to ultimately promote mitotic slippage. Indeed, it was observed that reducing the levels of the M43 isoform in different cancer cell lines increased mitotic arrest duration in the presence of anti-mitotic drugs. Conversely, changes that increase the levels of the M43 isoform relative to the full-length protein accelerated mitotic slippage and will likely negatively impact patient response. Thus, our findings provide potential avenues for the diagnosis and treatment of human cancers. The relative levels of Cdc20 isoforms may predict sensitivity to anti-mitotic drugs and could serve as a biomarker to designate appropriate patient populations for anti-mitotic therapies. Ultimately, an understanding of the regulatory pathways that control the differential translation of the Cdc20 isoforms may reveal ways to manipulate the relative levels of Cdc20 isoforms to make tumors more responsive to anti-mitotic drugs.

### Materials and Methods

### Cell Culture

HeLa, hTERT-RPE1, U2OS, and A549 cell lines were cultured in Dulbecco’s modified Eagle medium (DMEM) supplemented with 10% fetal bovine serum (FBS), 100 U/mL penicillin and streptomycin, and 2 mM L-glutamine at 37° C. with 5% CO₂. Doxycycline-inducible cell lines were cultured in medium containing FBS certified tetracycline free. Cas9 expression in inducible CRISPR/Cas9 cell lines was induced with 1 µg/ml doxycycline hyclate (##) at 24 hr interval for 2 days. All other doxycycline-inducible constructs were induced with 10 ng/ul doxycycline hyclate, unless indicated in figure legend. Other drugs used on human cells were Nocodazole (##, 330 nM), S-trityl-L-cysteine (STLC, ##, 10 µM), Taxol (##, 1 µM), GSK923295 (CENP-E inhibitor, ##, 100 nM), proTAME (APC/Ci, ##, 12 µM), AZ-3146 (Mps1i, ##, 4 µM) unless concentration indicated otherwise in figure legend. Cells were enriched in mitosis with treatment with 330 nM nocodazole for 16-17 hrs. Cell lines were tested monthly for mycoplasma contamination.

### Cell Line Generation

The cell lines used in this study are described in Table S2. The inducible CRISPR/Cas9 HeLa cell line was previously generated by transposition as described. A control sgRNA (Ctrl sgRNA, ##) or sgRNAs targeting different regions within the CDC20 gene (sgM1, ##; sgExon1, ##; sgExon3, ##) were cloned into the sgOpti plasmid (puro-resistant, Addgene ##) and introduced into the inducible CRISPR/Cas9 HeLa cell line by lentiviral transduction. Cells were selected with 0.5 µg/ml puromycin (##) for 5 days.

Stable clonal cell lines lacking the canonical full-length Cdc20 protein were obtained by transfecting HeLa cells with pX330-based plasmids [Cong 2013] expressing spCas9 and either the sgM1 (for ΔM1 mutant) or the sgExon1 guide RNA (for M1-stop mutant). pX330-based plasmids were transfected using Xtremegene-9 (##) together with a mCherry-expressing plasmid and mCherry-positive cells were fluorescence activated cell-sorted into 96 well plates with one cell per well. Clones were screened for successful gene editing and the CDC20 allele sequence information was determined by next-generation sequencing (Genewiz Amplicon-EZ).

pBABE derivatives containing empty IRES_EGFP or different Cdc20_IRES2_EGFP constructs were transfected with Effectene (Qiagen) along with VSVG packaging plasmid into 293-GP cells for generation of retrovirus as described [Morgenstern 1990]. Supernatant containing retrovirus was sterile-filtered, supplemented with 20 µg/mL polybrene (Millipore) and used to transduce inducible CRISPR/Cas9 HeLa expressing the sgExon3 guide RNA. After two days post-transduction, cells were selected with ## µg/ml hygromycin (##) for 10-14 days.

Doxycycline-inducible cell lines were generated by homology-directed insertion into the AAVS1 “safe-harbor” locus. Donor plasmid containing selection marker, the tetracycline-responsive promoter, the transgene, and reverse tetracycline-controlled transactivator flanked by AAVS1 homology arms [Qian 2014] was transfected into the indicated cell line using Effectene according to the manufacturer’s protocol with a pX330 based plasmid [Cong 2013] expressing both spCas9 and a guide RNA specific for the AAVS1 locus (pNM220, gRNA sequence - GGGGCCACTAGGGACAGGAT, SEQ ID NO: 10). After two days post-transduction, cells were selected with the indicated concentration of hygromycin (## µg/ml for HeLa and its derivatives, ## µg/ml for U2OS, and ## µg/ml for A549) for 10-14 days.

TABLE S2

Cell lines used

| | | | |
|---|---|---|---|
| Name | Description | Expression | Source |
| A549 | Parental | NA | Lab stock |
| HeLa | Parental | NA | Lab stock |
| hTERT-RPE1 | Parental | NA | Lab stock |
| U2OS | Parental | NA | Lab stock |
| cTT20 | HeLa tetON::spCas9 | Dox-inducible Cas9 | (McKinley and Cheeseman, 2017) |
| cKC443 | cTT20 / Ctrl sgRNA | Dox-inducible Cas9 | Lentiviral transduction of pKC328 |
| cMJ2 | cTT20_frameshift altATG/M43 -clone #1 | NA | Lentiviral transduction of pKMKO D4.1 + dox induction; clonal |
| cMJ3 | cTT20_frameshift altATG/M43 -clone #2 | NA | Lentiviral transduction of pKMKO D4.1 + dox induction; clonal |
| cMJ5 | cTT20 / sgExon3 | Dox-inducible Cas9 | Lentiviral transduction of pMJ7 |
| cMJ15 | cTT20_cdc20-mEGFP (heterozygous) | Dox-inducible Cas9 | Transient transfection of pMJ19 + pMJ27; clonal |
| cMJ35 | cMJ15_frameshift altATG/M43 | NA | Lentiviral transduction of pKMKO D4.1 + dox induction; clonal |
| cMJ46 | cMJ5 / Cdc20(1-499)_IRES2_EGFP | Dox-inducible Cas9/Constitutive retroviral | Retroviral transduction of pMJ39 |
| cMJ47 | cMJ5 / Cdc20(88-499)_IRES2_EGFP | Dox-inducible Cas9/Constitutive retroviral | Retroviral transduction of pMJ41 |
| cMJ90 | cTT20_frameshift M43/M88 -clone #1 | NA | Lentiviral transduction of pMJ32 + dox induction; clonal |
| cMJ91 | cTT20_frameshift M43/M88 -clone #2 | NA | Lentiviral transduction of pMJ32 + dox induction; clonal |
| cMJ94 | cMJ5 / Cdc20(43-499)_IRES2_EGFP | Dox-inducible Cas9/Constitutive retroviral | Retroviral transduction of pMJ40 |
| cMJ96 | cMJ5 / empty _IRES2_EGFP | Dox-inducible Cas9/Constitutive retroviral | Retroviral transduction of pMJ31 |
| cMJ159 | cTT20 / sgExon1 | Dox-inducible Cas9 | Lentiviral transduction of pMJ92 |
| cMJ200 | HeLa_M1-stop | NA | Transient transfection of pMJ94; clonal |
| cMJ222 | HeLa_ΔM1 | NA | Transient transfection of pMJ118; clonal |
| cMJ226 | cTT20 / sgM1 | Dox-inducible Cas9 | Retroviral transduction of pMJ141 |
| cMJ230 | cMJ200 / kozak _cdc20(WT)_3UTR | Dox-inducible AAVS1 | AAVS1 Safe-harbor insertion of pMJ143 |
| cMJ231 | cMJ200 / 5UTR_cdc20(WT)_3UTR | Dox-inducible AAVS1 | AAVS1 Safe-harbor insertion of pMJ144 |
| cMJ233 | cMJ200 / kozak_cdc20(M43L)_3UTR | Dox-inducible AAVS1 | AAVS1 Safe-harbor insertion of pMJ146 |
| cMJ234 | cMJ200 / kozak_cdc20(M88L)_3UTR | Dox-inducible AAVS1 | AAVS1 Safe-harbor insertion of pMJ147 |
| cMJ236 | cMJ200 / 5UTR_cdc20(altATGmutx2)_3U TR | Dox-inducible AAVS1 | AAVS1 Safe-harbor insertion of pMJ149 |
| cMJ241 | HeLa / kozak_cdc20(WT)_3UTR | Dox-inducible AAVS1 | AAVS1 Safe-harbor insertion of pMJ143 |
| cMJ242 | cMJ222 / kozak_cdc20(WT)_3UTR | Dox-inducible AAVS1 | AAVS1 Safe-harbor insertion of pMJ143 |
| cMJ253 | HeLa /5UTR_cdc20(WT)_3UTR | Dox-inducible AAVS1 | AAVS1 Safe-harbor insertion of pMJ144 |
| cMJ256 | HeLa / 5UTR_cdc20(altATGmutx2)_3U TR | Dox-inducible AAVS1 | AAVS1 Safe-harbor insertion of pMJ149 |
| cMJ261 | cMJ200 /kozak_cdc20(WT-low)_3UTR | Dox-inducible AAVS1 | Single-cell sort cMJ230; clonal |
| cMJ263 | cMJ200 / kozak_cdc20(WT-med)_3UTR | Dox-inducible AAVS1 | Single-cell sort cMJ230; clonal |
| cMJ264 | cMJ200 / kozak_cdc20(WT-high)_3UTR | Dox-inducible AAVS1 | Single-cell sort cMJ230; clonal |
| cMJ268 | HeLa / mEGFP-cdc20(1-499) | Dox-inducible AAVS1 | AAVS1 Safe-harbor insertion of pMJ160 |
| cMJ269 | HeLa / mEGFP-cdc20(43-499) | Dox-inducible AAVS1 | AAVS1 Safe-harbor insertion of pMJ161 |
| cMJ270 | HeLa / mEGFP-cdc20(88-499) | Dox-inducible AAVS1 | AAVS1 Safe-harbor insertion of pMJ162 |
| cMJ274 | HeLa / kozak_cdc20(ΔM1)_3UTR | Dox-inducible AAVS1 | AAVS1 Safe-harbor insertion of pMJ163 |
| cMJ275 | HeLa / kozak_cdc20(ΔM1 M43L)_3UTR | Dox-inducible AAVS1 | AAVS1 Safe-harbor insertion of pMJ164 |
| cMJ276 | HeLa / kozak_cdc20(ΔM1 M88L)_3UTR | Dox-inducible AAVS1 | AAVS1 Safe-harbor insertion of pMJ165 |
| cMJ278 | cMJ200 / kozak_cdc20(ΔM1)3UTR | Dox-inducible AAVS1 | AAVS1 Safe-harbor insertion of pMJ163 |
| cMJ279 | cMJ200 / kozak_cdc20(ΔM1 M43L)_3UTR | Dox-inducible AAVS1 | AAVS1 Safe-harbor insertion of pMJ164 |
| cMJ280 | cMJ200 / kozak_cdc20(ΔM1 M88L)_3UTR | Dox-inducible AAVS1 | AAVS1 Safe-harbor insertion of pMJ165 |
| cMJ281 | cMJ200 / kozak_cdc20(ΔM1 M43L M88L)_3UTR | Dox-inducible AAVS1 | AAVS1 Safe-harbor insertion of pMJ166 |
| cMJ285 | cMJ200 / 5UTR_cdc20(addATG)_3UTR | Dox-inducible AAVS1 | AAVS1 Safe-harbor insertion of pMJ175 |
| cMJ288 | HeLa / 5UTR_cdc20(M43L M88L)_3UTR | Dox-inducible AAVS1 | AAVS1 Safe-harbor insertion of pMJ176 |
| cMJ290 | U2OS / 5UTR_cdc20(WT)_3UTR | Dox-inducible AAVS1 | AAVS1 Safe-harbor insertion of pMJ144 |
| cMJ292 | U2OS / 5UTR_cdc20(M43L M88L)_3UTR | Dox-inducible AAVS1 | AAVS1 Safe-harbor insertion of pMJ176 |
| cMJ294 | A549 / 5UTR_cdc20(WT)_3UTR | Dox-inducible AAVS1 | AAVS1 Safe-harbor insertion of pMJ144 |
| cMJ296 | A549 / 5UTR_cdc20(M43L M88L)_3UTR | Dox-inducible AAVS1 | AAVS1 Safe-harbor insertion of pMJ176 |
| cMJ321 | cMJ200 / kozak_cdc20(optimal-Kozak)_3UTR | Dox-inducible AAVS1 | AAVS1 Safe-harbor insertion of pMJ186 |
| cMJ322 | cMJ200 / kozak_cdc20(anti-Kozak)_3UTR | Dox-inducible AAVS1 | AAVS1 Safe-harbor insertion of pMJ187 |

### RNAi Treatment and Gene Replacements

Custom siRNAs against Cdc20 (##), Cdh1 (##), and Mad2 (##) and a non-targeting control pool (D-001810-10) were obtained from Dharmacon. siRNAs were applied at a final concentration of 50 nM, unless indicated in the figure legend. 2.5 µl Lipofectamine RNAiMax (Invitrogen) was used per ml of final transfection medium. For gene replacements, transfection medium also contained the appropriate concentration of doxycycline hyclate to express the ectopic inducible construct. Transfection medium was changed after 6 hrs for time-lapse microscopy analyses or within 20 hrs for Western blot analyses.

### Immunofluorescence Microscopy

Cells for immunofluorescence were seeded on poly-L-lysine (Sigma-Aldrich) coated coverslips and treated with 330 nM nocodazole for 1.5 hr before pre-extraction and fixation. Cells were pre-extracted in PBS + 0.2% Triton X-100 for 1 min at 37° C. before fixation with PBS + 0.2% Triton X-100 + 4% formaldehyde at room temperature for 10 min. Coverslips were washed with PBS + 0.1% Triton X-100 and blocked in Abdil (20 mM Tris-HCl, 150 mM NaCl, 0.1% Triton X-100, 3% BSA, 0.1% NaN₃, pH 7.5) for 30 min. Immunostaining was performed by incubating coverslips with primary antibodies diluted in Abdil for 45 min at room temperature followed by 3 consecutive washes with PBS + 0.1% Triton X-100. Cy2-and Cy5-conjugated secondary antibodies (Jackson ImmunoResearch Laboratories) were diluted 1:500 in Abdil together with ## µg/ml Hoechst-33342 (Sigma-Aldrich) and incubated with coverslips for 45 min. After washing, coverslips were mounted using Prolong Gold (##) and allowed to cure overnight.

The following primary antibodies were used: anti-centromere antibodies, ACA (1:200, Antibodies Inc, #15-234), Mad2 (1:1000, Kops Lab), Bub1 (1:200, Abcam, ab54893). Images were acquired on a DeltaVision Core deconvolution microscope (Applied Precision) equipped with a CoolSnap HQ2 charge-coupled device camera and deconvolved where appropriate using the Softworx software. Z-sections were acquired at 0.2 µm steps using a Plan Apo 100X/1.4 NA objective and appropriate fluorescence filters. Image analysis was performed in Fiji (ImageJ, NIH).

### Western Blot

Cells were treated with 330 nM nocodazole for 16 hrs before harvesting for Western blot analysis. Cells were washed with PBS and then lysed on ice for 30 min in fresh urea lysis buffer (##). Cellular debris was removed by centrifugation. Protein concentrations in each sample were measured using Bradford reagent (Bio-Rad), and sample concentrations were normalized before addition of 2X Laemmli buffer. Lysates were heated at 95° C. for 5 min, separated by SDS-PAGE, and transferred to PVDF (##) membrane. Membranes were blocked for 1 hr in Blocking Buffer (2% milk in PBS + 0.05% Tween-20). Primary antibodies were diluted in 0.2% milk in PBS + 0.05% Tween-20 + 0.2% NaN₃ and applied to the membrane overnight at 4° C. HRP-conjugated secondary antibodies (##) were diluted 1:1000 in 0.2% milk in PBS + 0.05% Tween-20 and applied to the membrane for 1 hr at room temperature. After washing in PBS + 0.05% Tween-20, Clarity enhanced chemiluminescence substrate (Bio-Rad) was added to the membrane according to the manufacturer’s instructions. Membranes were imaged with a KwikQuant Imager (Kindle Biosciences).

### Lambda Phosphatase Treatment

HeLa cells were treated with 330 nM nocodazole overnight and mitotic cells were harvested by shake-off. Cells were washed with PBS and then lysed on ice for 45 min in HEPES/Triton X-100 lysis buffer (##). Cellular debris was removed by centrifugation and the resulting lysate was split into three parts. One sample was left untreated and the other two were supplemented with 1X Protein MetalloPhosphatase buffer (New England Biolabs) and 1 mM MnCl₂ only or together with Lambda Protein Phosphatase (New England Biolabs). After incubation at 30° C. for 30 min, reactions were stopped by addition of 2X Laemmli buffer. Samples were analyzed by SDS-PAGE and Western blot.

### Cell Synchronization Using Single Thymidine Arrest

HeLa cells were first arrested in S phase using 2.5 mM thymidine for 22 hrs and then washed and released into medium without thymidine. Cells were collected at various time points after the single thymidine release. At 6 hrs post-release, 330 nM nocodazole was added to arrest cells in mitosis. Harvested cells were analyzed by SDS-PAGE and Western blot.

### Time-Lapse Experiments for Mitotic Timing

Cells were first seeded in 12-well polymer-bottomed plates (Cellvis, ##) and treated as indicated in the figure legends. Cells were later moved to CO₂-independent media (##) before imaging at 37° C. Phase contrast images were acquired on a Nikon eclipse microscope equipped with a charge-coupled device (CCD) camera (Clara, Andor) or a CMOS camera (##) using a Plan Fluor 20X/0.5 NA objective at either 5 min or 10 min intervals. Time-lapse movies were analyzed using Fiji (ImageJ, NIH), ilastik (##) and CellProfiler (##). Image brightness and contrast was adjusted in Fiji, pixel-based classification was performed on individual images using ilastik and the resulting probability map images were processed by CellProfiler to identify and track mitotic cells. Each mitotic cell was then confirmed manually and the mitotic duration was determined as the time from cell rounding at mitotic entry to cell flattening after mitotic exit.

### Live-Cell Fluorescence Imaging

For live-cell fluorescence imaging, cells were seeded into 8-well glass-bottomed chambers (Ibidi) and moved into CO₂-independent media (##) before imaging at 37° C. DNA was stained with ## µg/ml Hoechst. Cells were imaged directly or after 1 hr incubation with 330 nM nocodazole. Images were acquired on a DeltaVision Core deconvolution microscope (Applied Precision) equipped with a CoolSnap HQ2 charge-coupled device camera and deconvolved using the Softworx software. Z-sections were acquired at 0.5 µm steps using a Plan Apo 100X/1.4 NA objective and appropriate fluorescence filters. Image analysis was performed in Fiji (ImageJ, NIH).

### Mitotic Index Determination From Microscopy

Cells were seeded in 12-well polymer-bottomed plates (Cellvis, ##) and treated as indicated in the figure legends before fixing in PBS + 4% formaldehyde for 10 min at room temperature. After washing with PBS, cells were incubated in Abdil containing ## µg/ml Hoechst for 30 min. Cells were washed with PBS and stored in Abdil only until ready to image. Images were aquired on a Nikon eclipse microscope equipped with a charge-coupled device (CCD) camera (Clara; Andor) using a Plan Fluor 20X/0.5 NA objective and appropriate fluorescence filters. Image analysis was performed in Fiji (ImageJ, NIH). The mitotic index was determined by scoring the number of mitotic cells with condensed DNA and dividing by the total number of cells.

### Mitotic Index Determination by Flow Cytometry

Cells were collected by incubation for 10 min in PBS + 5 mM EDTA, washed once in PBS, then fixed in PBS + 2% formaldehyde for 10 min at room temperature. Cells were blocked in Abdil for 30 min followed by immunostaining for phosphorylated S10 on histone 3 followed by Cy-5 conjugated secondary antibody. The proportion of GFP-positive single cells also staining positive for H3pS10 was determined on an LSRFortessa (BD Biosciences) flow cytometer and analyzed with FlowJo software (##).

### GFP Immunoprecipitation and Mass Spectrometry

IP-MS experiments were performed as described previously (Cheeseman and Desai 2005). Harvested cells were washed in PBS and resuspended 1:1 in 1X Lysis Buffer (50 mM HEPES, 1 mM EGTA, 1 mM MgCl2, 100 mM KCL, 10% glycerol, pH 7.4) then drop frozen in liquid nitrogen. Cells were thawed after addition of an equal volume of 1.5X lysis buffer supplemented with 0.075% Nonidet P-40, 1X Complete EDTA-free protease inhibitor cocktail (Roche), 1 mM PMSF, 20 mM beta-glycerophosphate, 1 mM sodium fluoride, and 0.4 mM sodium orthovanadate. Cells were lysed by sonication and cleared by centrifugation. The supernatant was mixed with Protein A beads coupled to rabbit anti-GFP antibody (Cheeseman lab) and rotated at 4° C. for 1 hr. Beads were washed five times in Wash Buffer (50 mM HEPES, 1 mM EGTA, 1 mM MgCl2, 300 mM KCl, 10% glycerol, 0.05% NP-40, 1 mM DTT, 10 µg/mL leupeptin/pepstatin/chymostatin, pH 7.4). After a final wash in Wash Buffer without detergent, bound protein was eluted with 100 mM glycine pH2.6. Eluted proteins were precipitated by addition of ⅕^{th} volume trichloroacetic acid at 4° C. overnight. Precipitated proteins were reduced with TCEP, alkylated with iodoacetamide, and digested with mass-spectrometry grade Lys-C and trypsin or Lys-C alone (Promega). Digested peptides were analyzed on an ### mass spectrometer (Thermo Fisher) coupled with a reverse phase gradient over C18 resin. Data were analyzed using SEQUEST.

### M88Ac Antibody Generation

The M88Ac antibody was generated against a synthesized acetylated-peptide with the following amino acid sequence: Ac-## (New England Peptide; Covance). Serum from immunized rabbit was depleted against a non-acetylated spanning peptide and affinity-purified against the acetylated peptide.

### MTT Viability Assay

HeLa or M1-stop cells were seeded at a density of 2,000 cells per well in 96-well plates and subsequently cultured for 72 hrs in triplicate with increasing concentrations of the indicated anti-mitotic drug. After 72 hrs incubation, the medium was removed and the cells were stained with 2.5 mg/ml MTT (3-(4,5-dimethylthiazol-2-yl)-2,5-diphenyltetrazolium bromide) in medium without serum for 3 hrs. Formazan crystals formed by the cells were then dissolved in 4 mM HCl and 0.1% NP-40 in isopropanol for 15 min. The absorbance was read at 570 nm on a Multiskan GO plate reader (Thermo Scientific), using 650 nm as reference wavelength.

### Example 2

Identifying novel therapeutic targets for the treatment of human cancers associated with impaired spindle assembly checkpoint activity

Making effective choices for anti-cancer therapies in a patient-specific manner is critical. Such therapies should ideally be “tumor-specific” to minimize toxicity in clinical applications. Many cancer cells have been reported to display compromised spindle assembly checkpoint activity. Such cancers may be less sensitive to anti-mitotic cancer therapies that rely on mitotic arrest due to prolonged checkpoint activation, thus limiting treatment options. To reveal which treatments would be most potent in those cancer cells with defective SAC activity, Applicants will apply the genetic tool known as synthetic lethality, which occurs when the simultaneous loss of two dispensable genes leads to a loss of viability. This will allow the identification of non-essential genes that only become essential in the absence of the spindle assembly checkpoint. Such genes may provide potential targets for the development of potent therapies against cancer cells with defective SAC activity. These synthetic lethal screens are particularly well suited for me given my rigorous training as a geneticist and my experience undertaking genetic screens.

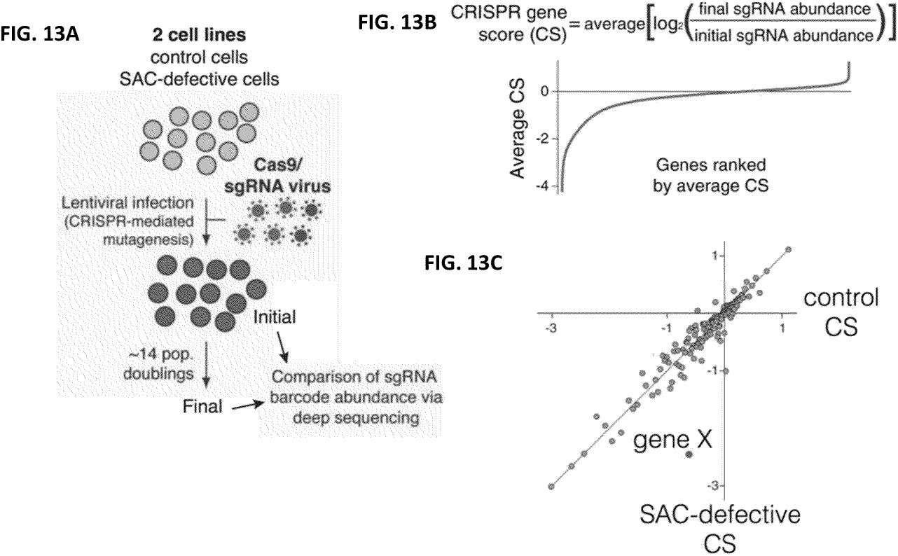

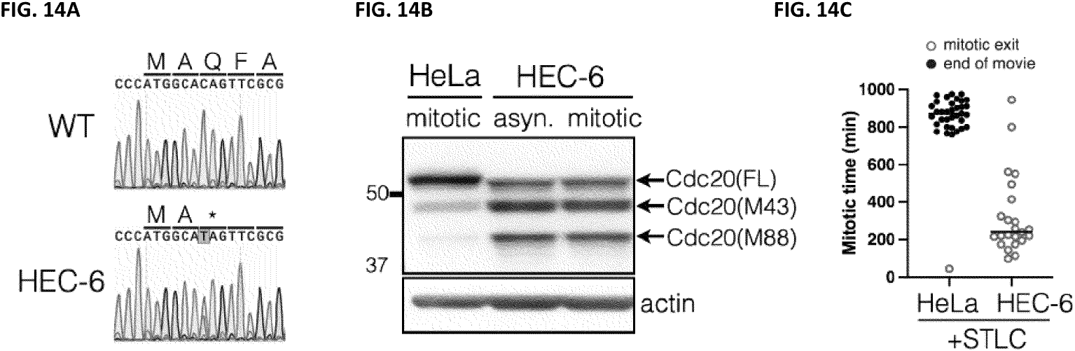

In these synthetic lethal screens, Applicants will apply functional genomic approaches to identify potential novel therapeutic targets for the treatment of human cancers associated with impaired SAC activity. To mimic such cancers, Applicants have constructed stable genetic knockouts and hypomorphic mutant human cell lines that are viable, but bypass the mitotic arrest due to spindle assembly checkpoint activation upon treatment with nocodazole. These SAC-defective cell lines include the stable cell line expressing only the short, checkpoint-insensitive Cdc20 isoforms. Applicants propose to identify genes that display synthetic lethal interactions with the spindle assembly checkpoint by performing pooled genome-wide loss-of-function CRISPR-Cas9 screens with these SAC-defective cell lines. Briefly, Applicants will infect control human cells or SAC-defective cells with a lentiviral pool harboring single-guide RNAs (sgRNAs) targeting 18,166 human genes with approximately 10 sgRNAs each [18]. Using deep sequencing of the sgRNA barcodes, Applicants will monitor the changes in sgRNA abundance between the initial and final cell populations following growth with Cas9 cleavage (FIGS. 13A-B). Genes targeted by sgRNAs that are selectively depleted in SAC-defective cells compared to the control cells represent factors that become important for cellular viability in the absence of the spindle assembly checkpoint (FIG. 13C). In addition to defining targets for cancer treatment, these factors may uncover new regulators and biological pathways involved in chromosome segregation. More importantly, the identified factors will only be essential in cells with impaired SAC function, such as cancer cells, and thus may provide novel targets for the design of specific anti-tumor strategies that preferentially kill tumor cells.

The functional genomic approach described above will allow the high-throughput identification of non-essential factors (both established and unknown) that become essential when the spindle assembly checkpoint is compromised. To define the candidates that are best suited for drug treatment, providing an “Achilles heel” for cancer cells, Applicants will analyze specific screen hits to define their molecular properties and mitotic behavior. Applicants will first assess the phenotypic consequences of depleting candidate hits by transfecting synthetic guide RNAs in control cells or SAC-defective cells expressing Cas9 and immunostaining for microtubules and DNA. Applicants will then focus on genes with depletion phenotypes suggesting a role in chromosome segregation: inactivation of these genes would result in similar defects in chromosome alignment and segregation as mutants of established kinetochore components. In addition, Applicants will benefit from recent work in the Cheeseman lab that defined the phenotypic “fingerprints” that result from compromising specific cell division processes, thus allowing Applicants to quickly implicate novel factors in a given process. Applicants will validate the candidate genes by constructing individual inducible or stable knockout cell lines and performing complementation assays with transgenes in which the sequence complementary to the sgRNA is mutated to prevent Cas9 cleavage. Additionally, Applicants will assess the localization of the corresponding protein by fluorescence microscopy to determine a potential role in cell division or chromosome segregation. Applicants will subject candidate novel cell division components to further in-depth analyses using cell biological and biochemical approaches. Validated hits will also provide novel targets for the design of specific anti-tumor strategies that preferentially kill tumor cells. Applicants anticipate that these studies will reveal new insights and approaches for the control and treatment of various types of human cancers, particularly those human cancers associated with impaired SAC activity such as some lung and ovarian cancers.

### Sequences

SEQ ID NO: 1- CDC20 (human WT) DNA>>ATGGCACAGTTCG
CGTTCGAGAGTGACCTGCACTCGCTGCTTCAGCTGGATGCACCCATCCCC
AATGCACCCCCTGCGCGCTGGCAGCGCAAAGCCAAGGAAGCCGCAGGCCC
GGCCCCCTCACCCATGCGGGCCGCCAACCGATCCCACAGCGCCGGCAGGA
CTCCGGGCCGAACTCCTGGCAAATCCAGTTCCAAGGTTCAGACCACTCCT
AGCAAACCTGGCGGTGACCGCTATATCCCCCATCGCAGTGCTGCCCAGAT
GGAGGTGGCCAGCTTCCTCCTGAGCAAGGAGAACCAGCCTGAAAACAGCC
AGACGCCCACCAAGAAGGAACATCAGAAAGCCTGGGCTTTGAACCTGAAC
GGTTTTGATGTAGAGGAAGCCAAGATCCTTCGGCTCAGTGGAAAACCACA
AAATGCGCCAGAGGGTTATCAGAACAGACTGAAAGTACTCTACAGCCAAA
AGGCCACTCCTGGCTCCAGCCGGAAGACCTGCCGTTACATTCCTTCCCTG
CCAGACCGTATCCTGGATGCGCCTGAAATCCGAAATGACTATTACCTGAA
CCTTGTGGATTGGAGTTCTGGGAATGTACTGGCCGTGGCACTGGACAACA
GTGTGTACCTGTGGAGTGCAAGCTCTGGTGACATCCTGCAGCTTTTGCAA
ATGGAGCAGCCTGGGGAATATATATCCTCTGTGGCCTGGATCAAAGAGGG
CAACTACTTGGCTGTGGGCACCAGCAGTGCTGAGGTGCAGCTATGGGATG
TGCAGCAGCAGAAACGGCTTCGAAATATGACCAGTCACTCTGCCCGAGTG
GGCTCCCTAAGCTGGAACAGCTATATCCTGTCCAGTGGTTCACGTTCTGG
CCACATCCACCACCATGATGTTCGGGTAGCAGAACACCATGTGGCCACAC
TGAGTGGCCACAGCCAGGAAGTGTGTGGGCTGCGCTGGGCCCCAGATGGA
CGACATTTGGCCAGTGGTGGTAATGATAACTTGGTCAATGTGTGGCCTAG
TGCTCCTGGAGAGGGTGGCTGGGTTCCTCTGCAGACATTCACCCAGCATC
AAGGGGCTGTCAAGGCCGTAGCATGGTGTCCCTGGCAGTCCAATGTCCTG
GCAACAGGAGGGGGCACCAGTGATCGACACATTCGCATCTGGAATGTGTG
CTCTGGGGCCTGTCTGAGTGCCGTGGATGCCCATTCCCAGGTGTGCTCCA
TCCTCTGGTCTCCCCATTACAAGGAGCTCATCTCAGGCCATGGCTTTGCA
CAGAACCAGCTAGTTATTTGGAAGTACCCAACCATGGCCAAGGTGGCTGA
ACTCAAAGGTCACACATCCCGGGTCCTGAGTCTGACCATGAGCCCAGATG
GGGCCACAGTGGCATCCGCAGCAGCAGATGAGACCCTGAGGCTATGGCGC
TGTTTTGAGTTGGACCCTGCGCGGCGGCGGGAGCGGGAGAAGGCCAGTGC
AGCCAAAAGCAGCCTCATCCACCAAGGCATCCGCTGA

SEQ ID NO: 2- CDC20 (human WT) Prot>>>MAQFAFESDLH
SLLQLDAPIPNAPPARWQRKAKEAAGPAPSPMRAANRSHSAGRTPGRTPG
KSSSKVQTTPSKPGGDRYIPHRSAAQMEVASFLLSKENQPENSQTPTKKE
HQKAWALNLNGFDVEEAKILRLSGKPQNAPEGYQNRLKVLYSQKATPGSS
RKTCRYIPSLPDRILDAPEIRNDYYLNLVDWSSGNVLAVALDNSVYLWSA
SSGDILQLLQMEQPGEYISSVAWIKEGNYLAVGTSSAEVQLWDVQQQKRL
RNMTSHSARVGSLSWNSYILSSGSRSGHIHHHDVRVAEHHVATLSGHSQE
VCGLRWAPDGRHLASGGNDNLVNVWPSAPGEGGWVPLQTFTQHQGAVKAV
AWCPWQSNVLATGGGTSDRHIRIWNVCSGACLSAVDAHSQVCSILWSPHY
KELISGHGFAQNQLVIWKYPTMAKVAELKGHTSRVLSLTMSPDGATVASA
AADETLRLWRCFELDPARRREREKASAAKSSLIHQGIR∗

SEQ ID NO: 3- CDC20 truncation 43-499 DNA>>ATGCGG
GCCGCCAACCGATCCCACAGCGCCGGCAGGACTCCGGGCCGAACTCCTGG
CAAATCCAGTTCCAAGGTTCAGACCACTCCTAGCAAACCTGGCGGTGACC
GCTATATCCCCCATCGCAGTGCTGCCCAGATGGAGGTGGCCAGCTTCCTC
CTGAGCAAGGAGAACCAGCCTGAAAACAGCCAGACGCCCACCAAGAAGGA
ACATCAGAAAGCCTGGGCTTTGAACCTGAACGGTTTTGATGTAGAGGAAG
CCAAGATCCTTCGGCTCAGTGGAAAACCACAAAATGCGCCAGAGGGTTAT
CAGAACAGACTGAAAGTACTCTACAGCCAAAAGGCCACTCCTGGCTCCAG
CCGGAAGACCTGCCGTTACATTCCTTCCCTGCCAGACCGTATCCTGGATG
CGCCTGAAATCCGAAATGACTATTACCTGAACCTTGTGGATTGGAGTTCT
GGGAATGTACTGGCCGTGGCACTGGACAACAGTGTGTACCTGTGGAGTGC
AAGCTCTGGTGACATCCTGCAGCTTTTGCAAATGGAGCAGCCTGGGGAAT
ATATATCCTCTGTGGCCTGGATCAAAGAGGGCAACTACTTGGCTGTGGGC
ACCAGCAGTGCTGAGGTGCAGCTATGGGATGTGCAGCAGCAGAAACGGCT
TCGAAATATGACCAGTCACTCTGCCCGAGTGGGCTCCCTAAGCTGGAACA
GCTATATCCTGTCCAGTGGTTCACGTTCTGGCCACATCCACCACCATGAT
GTTCGGGTAGCAGAACACCATGTGGCCACACTGAGTGGCCACAGCCAGGA
AGTGTGTGGGCTGCGCTGGGCCCCAGATGGACGACATTTGGCCAGTGGTG
GTAATGATAACTTGGTCAATGTGTGGCCTAGTGCTCCTGGAGAGGGTGGC
TGGGTTCCTCTGCAGACATTCACCCAGCATCAAGGGGCTGTCAAGGCCGT
AGCATGGTGTCCCTGGCAGTCCAATGTCCTGGCAACAGGAGGGGGCACCA
GTGATCGACACATTCGCATCTGGAATGTGTGCTCTGGGGCCTGTCTGAGT
GCCGTGGATGCCCATTCCCAGGTGTGCTCCATCCTCTGGTCTCCCCATTA
CAAGGAGCTCATCTCAGGCCATGGCTTTGCACAGAACCAGCTAGTTATTT
GGAAGTACCCAACCATGGCCAAGGTGGCTGAACTCAAAGGTCACACATCC
CGGGTCCTGAGTCTGACCATGAGCCCAGATGGGGCCACAGTGGCATCCGC
AGCAGCAGATGAGACCCTGAGGCTATGGCGCTGTTTTGAGTTGGACCCTG
CGCGGCGGCGGGAGCGGGAGAAGGCCAGTGCAGCCAAAAGCAGCCTCATC
CACCAAGGCATCCGCTGA

SEQ ID NO: 4- CDC20 truncation 43-499 Prot>>>MRAA
NRSHSAGRTPGRTPGKSSSKVQTTPSKPGGDRYIPHRSAAQMEVASFLLS
KENQPENSQTPTKKEHQKAWALNLNGFDVEEAKILRLSGKPQNAPEGYQN
RLKVLYSQKATPGSSRKTCRYIPSLPDRILDAPEIRNDYYLNLVDWSSGN
VLAVALDNSVYLWSASSGDILQLLQMEQPGEYISSVAWIKEGNYLAVGTS
SAEVQLWDVQQQKRLRNMTSHSARVGSLSWNSYILSSGSRSGHIHHHDVR
VAEHHVATLSGHSQEVCGLRWAPDGRHLASGGNDNLVNVWPSAPGEGGWV
PLQTFTQHQGAVKAVAWCPWQSNVLATGGGTSDRHIRIWNVCSGACLSAV
DAHSQVCSILWSPHYKELISGHGFAQNQLVIWKYPTMAKVAELKGHTSRV
LSLTMSPDGATVASAAADETLRLWRCFELDPARRREREKASAAKSSLIHQ
GIR∗

SEQ ID NO: 5- CDC20 truncation 88-499 DNA>>ATGGAG
GTGGCCAGCTTCCTCCTGAGCAAGGAGAACCAGCCTGAAAACAGCCAGAC
GCCCACCAAGAAGGAACATCAGAAAGCCTGGGCTTTGAACCTGAACGGTT
TTGATGTAGAGGAAGCCAAGATCCTTCGGCTCAGTGGAAAACCACAAAAT
GCGCCAGAGGGTTATCAGAACAGACTGAAAGTACTCTACAGCCAAAAGGC
CACTCCTGGCTCCAGCCGGAAGACCTGCCGTTACATTCCTTCCCTGCCAG
ACCGTATCCTGGATGCGCCTGAAATCCGAAATGACTATTACCTGAACCTT
GTGGATTGGAGTTCTGGGAATGTACTGGCCGTGGCACTGGACAACAGTGT
GTACCTGTGGAGTGCAAGCTCTGGTGACATCCTGCAGCTTTTGCAAATGG
AGCAGCCTGGGGAATATATATCCTCTGTGGCCTGGATCAAAGAGGGCAAC
TACTTGGCTGTGGGCACCAGCAGTGCTGAGGTGCAGCTATGGGATGTGCA
GCAGCAGAAACGGCTTCGAAATATGACCAGTCACTCTGCCCGAGTGGGCT
CCCTAAGCTGGAACAGCTATATCCTGTCCAGTGGTTCACGTTCTGGCCAC
ATCCACCACCATGATGTTCGGGTAGCAGAACACCATGTGGCCACACTGAG
TGGCCACAGCCAGGAAGTGTGTGGGCTGCGCTGGGCCCCAGATGGACGAC
ATTTGGCCAGTGGTGGTAATGATAACTTGGTCAATGTGTGGCCTAGTGCT
CCTGGAGAGGGTGGCTGGGTTCCTCTGCAGACATTCACCCAGCATCAAGG
GGCTGTCAAGGCCGTAGCATGGTGTCCCTGGCAGTCCAATGTCCTGGCAA
CAGGAGGGGGCACCAGTGATCGACACATTCGCATCTGGAATGTGTGCTCT
GGGGCCTGTCTGAGTGCCGTGGATGCCCATTCCCAGGTGTGCTCCATCCT
CTGGTCTCCCCATTACAAGGAGCTCATCTCAGGCCATGGCTTTGCACAGA
ACCAGCTAGTTATTTGGAAGTACCCAACCATGGCCAAGGTGGCTGAACTC
AAAGGTCACACATCCCGGGTCCTGAGTCTGACCATGAGCCCAGATGGGGC
CACAGTGGCATCCGCAGCAGCAGATGAGACCCTGAGGCTATGGCGCTGTT
TTGAGTTGGACCCTGCGCGGCGGCGGGAGCGGGAGAAGGCCAGTGCAGCC
AAAAGCAGCCTCATCCACCAAGGCATCCGCTGA

SEQ ID NO: 6- CDC20 truncation 88-499 Prot>>>MEVA
SFLLSKENQPENSQTPTKKEHQKAWALNLNGFDVEEAKILRLSGKPQNAP
EGYQNRLKVLYSQKATPGSSRKTCRYIPSLPDRILDAPEIRNDYYLNLVD
WSSGNVLAVALDNSVYLWSASSGDILQLLQMEQPGEYISSVAWIKEGNYL
AVGTSSAEVQLWDVQQQKRLRNMTSHSARVGSLSWNSYILSSGSRSGHIH
HHDVRVAEHHVATLSGHSQEVCGLRWAPDGRHLASGGNDNLVNVWPSAPG
EGGWVPLQTFTQHQGAVKAVAWCPWQSNVLATGGGTSDRHIRIWNVCSGA
CLSAVDAHSQVCSILWSPHYKELISGHGFAQNQLVIWKYPTMAKVAELKG
HTSRVLSLTMSPDGATVASAAADETLRLWRCFELDPARRREREKASAAKS
SLIHQGIR*

SEQ ID NO: 7- DeltaM1 DNA (homozygous)>>GCACAGTTC
GCGTTCGAGAGTGACCTGCACTCGCTGCTTCAGCTGGATGCACCCATCCC
CAATGCACCCCCTGCGCGCTGGCAGCGCAAAGCCAAGGAAGCCGCAGGCC
CGGCCCCCTCACCCATGCGGGCCGCCAACCGATCCCACAGCGCCGGCAGG
ACTCCGGGCCGAACTCCTGGCAAATCCAGTTCCAAGGTTCAGACCACTCC
TAGCAAACCTGGCGGTGACCGCTATATCCCCCATCGCAGTGCTGCCCAGA
TGGAGGTGGCCAGCTTCCTCCTGAGCAAGGAGAACCAGCCTGAAAACAGC
CAGACGCCCACCAAGAAGGAACATCAGAAAGCCTGGGCTTTGAACCTGAA
CGGTTTTGATGTAGAGGAAGCCAAGATCCTTCGGCTCAGTGGAAAACCAC
AAAATGCGCCAGAGGGTTATCAGAACAGACTGAAAGTACTCTACAGCCAA
AAGGCCACTCCTGGCTCCAGCCGGAAGACCTGCCGTTACATTCCTTCCCT
GCCAGACCGTATCCTGGATGCGCCTGAAATCCGAAATGACTATTACCTGA
ACCTTGTGGATTGGAGTTCTGGGAATGTACTGGCCGTGGCACTGGACAAC
AGTGTGTACCTGTGGAGTGCAAGCTCTGGTGACATCCTGCAGCTTTTGCA
AATGGAGCAGCCTGGGGAATATATATCCTCTGTGGCCTGGATCAAAGAGG
GCAACTACTTGGCTGTGGGCACCAGCAGTGCTGAGGTGCAGCTATGGGAT
GTGCAGCAGCAGAAACGGCTTCGAAATATGACCAGTCACTCTGCCCGAGT
GGGCTCCCTAAGCTGGAACAGCTATATCCTGTCCAGTGGTTCACGTTCTG
GCCACATCCACCACCATGATGTTCGGGTAGCAGAACACCATGTGGCCACA
CTGAGTGGCCACAGCCAGGAAGTGTGTGGGCTGCGCTGGGCCCCAGATGG
ACGACATTTGGCCAGTGGTGGTAATGATAACTTGGTCAATGTGTGGCCTA
GTGCTCCTGGAGAGGGTGGCTGGGTTCCTCTGCAGACATTCACCCAGCAT
CAAGGGGCTGTCAAGGCCGTAGCATGGTGTCCCTGGCAGTCCAATGTCCT
GGCAACAGGAGGGGGCACCAGTGATCGACACATTCGCATCTGGAATGTGT
GCTCTGGGGCCTGTCTGAGTGCCGTGGATGCCCATTCCCAGGTGTGCTCC
ATCCTCTGGTCTCCCCATTACAAGGAGCTCATCTCAGGCCATGGCTTTGC
ACAGAACCAGCTAGTTATTTGGAAGTACCCAACCATGGCCAAGGTGGCTG
AACTCAAAGGTCACACATCCCGGGTCCTGAGTCTGACCATGAGCCCAGAT
GGGGCCACAGTGGCATCCGCAGCAGCAGATGAGACCCTGAGGCTATGGCG
CTGTTTTGAGTTGGACCCTGCGCGGCGGCGGGAGCGGGAGAAGGCCAGTG
CAGCCAAAAGCAGCCTCATCCACCAAGGCATCCGCTGA

SEQ ID NO: 8- M1-stop mutant DNA (allele1)>>ATGGC
ACAGTTCGCGTTCGAGAGTGACCTGCACTCGCTGCTTCACAGGGTTTCTC
CATGTTGGTCAGGCTGATCTTGAACTCCCAACCTCAGGTGAAGCTGGATG
CACCCATCCCCAATGCACCCCCTGCGCGCTGGCAGCGCAAAGCCAAGGAA
GCCGCAGGCCCGGCCCCCTCACCCATGCGGGCCGCCAACCGATCCCACAG
CGCCGGCAGGACTCCGGGCCGAACTCCTGGCAAATCCAGTTCCAAGGTTC
AGACCACTCCTAGCAAACCTGGCGGTGACCGCTATATCCCCCATCGCAGT
GCTGCCCAGATGGAGGTGGCCAGCTTCCTCCTGAGCAAGGAGAACCAGCC
TGAAAACAGCCAGACGCCCACCAAGAAGGAACATCAGAAAGCCTGGGCTT
TGAACCTGAACGGTTTTGATGTAGAGGAAGCCAAGATCCTTCGGCTCAGT
GGAAAACCACAAAATGCGCCAGAGGGTTATCAGAACAGACTGAAAGTACT
CTACAGCCAAAAGGCCACTCCTGGCTCCAGCCGGAAGACCTGCCGTTACA
TTCCTTCCCTGCCAGACCGTATCCTGGATGCGCCTGAAATCCGAAATGAC
TATTACCTGAACCTTGTGGATTGGAGTTCTGGGAATGTACTGGCCGTGGC
ACTGGACAACAGTGTGTACCTGTGGAGTGCAAGCTCTGGTGACATCCTGC
AGCTTTTGCAAATGGAGCAGCCTGGGGAATATATATCCTCTGTGGCCTGG
ATCAAAGAGGGCAACTACTTGGCTGTGGGCACCAGCAGTGCTGAGGTGCA
GCTATGGGATGTGCAGCAGCAGAAACGGCTTCGAAATATGACCAGTCACT
CTGCCCGAGTGGGCTCCCTAAGCTGGAACAGCTATATCCTGTCCAGTGGT
TCACGTTCTGGCCACATCCACCACCATGATGTTCGGGTAGCAGAACACCA
TGTGGCCACACTGAGTGGCCACAGCCAGGAAGTGTGTGGGCTGCGCTGGG
CCCCAGATGGACGACATTTGGCCAGTGGTGGTAATGATAACTTGGTCAAT
GTGTGGCCTAGTGCTCCTGGAGAGGGTGGCTGGGTTCCTCTGCAGACATT
CACCCAGCATCAAGGGGCTGTCAAGGCCGTAGCATGGTGTCCCTGGCAGT
CCAATGTCCTGGCAACAGGAGGGGGCACCAGTGATCGACACATTCGCATC
TGGAATGTGTGCTCTGGGGCCTGTCTGAGTGCCGTGGATGCCCATTCCCA
GGTGTGCTCCATCCTCTGGTCTCCCCATTACAAGGAGCTCATCTCAGGCC
ATGGCTTTGCACAGAACCAGCTAGTTATTTGGAAGTACCCAACCATGGCC
AAGGTGGCTGAACTCAAAGGTCACACATCCCGGGTCCTGAGTCTGACCAT
GAGCCCAGATGGGGCCACAGTGGCATCCGCAGCAGCAGATGAGACCCTGA
GGCTATGGCGCTGTTTTGAGTTGGACCCTGCGCGGCGGCGGGAGCGGGAG
AAGGCCAGTGCAGCCAAAAGCAGCCTCATCCACCAAGGCATCCGCTGA

SEQ ID NO: 9- M1-stop mutant DNA (allele2)>>ATGGC
ACAGTTCGCGTTCGAGAGTGACCTGCACTCGCTGCTTCGATTGGGAAGAG
AATAGCAGGCATGCTGGGGAGCGGCCGCAGGAACCCCTAGTGATGGAGTT
GGCCACTCCCTCTCTGCGCGCTCGCTCGCTCACTGAGGCCGCCCGCTGGA
TGCACCCATCCCCAATGCACCCCCTGCGCGCTGGCAGCGCAAAGCCAAGG
AAGCCGCAGGCCCGGCCCCCTCACCCATGCGGGCCGCCAACCGATCCCAC
AGCGCCGGCAGGACTCCGGGCCGAACTCCTGGCAAATCCAGTTCCAAGGT
TCAGACCACTCCTAGCAAACCTGGCGGTGACCGCTATATCCCCCATCGCA
GTGCTGCCCAGATGGAGGTGGCCAGCTTCCTCCTGAGCAAGGAGAACCAG
CCTGAAAACAGCCAGACGCCCACCAAGAAGGAACATCAGAAAGCCTGGGC
TTTGAACCTGAACGGTTTTGATGTAGAGGAAGCCAAGATCCTTCGGCTCA
GTGGAAAACCACAAAATGCGCCAGAGGGTTATCAGAACAGACTGAAAGTA
CTCTACAGCCAAAAGGCCACTCCTGGCTCCAGCCGGAAGACCTGCCGTTA
CATTCCTTCCCTGCCAGACCGTATCCTGGATGCGCCTGAAATCCGAAATG
ACTATTACCTGAACCTTGTGGATTGGAGTTCTGGGAATGTACTGGCCGTG
GCACTGGACAACAGTGTGTACCTGTGGAGTGCAAGCTCTGGTGACATCCT
GCAGCTTTTGCAAATGGAGCAGCCTGGGGAATATATATCCTCTGTGGCCT
GGATCAAAGAGGGCAACTACTTGGCTGTGGGCACCAGCAGTGCTGAGGTG
CAGCTATGGGATGTGCAGCAGCAGAAACGGCTTCGAAATATGACCAGTCA
CTCTGCCCGAGTGGGCTCCCTAAGCTGGAACAGCTATATCCTGTCCAGTG
GTTCACGTTCTGGCCACATCCACCACCATGATGTTCGGGTAGCAGAACAC
CATGTGGCCACACTGAGTGGCCACAGCCAGGAAGTGTGTGGGCTGCGCTG
GGCCCCAGATGGACGACATTTGGCCAGTGGTGGTAATGATAACTTGGTCA
ATGTGTGGCCTAGTGCTCCTGGAGAGGGTGGCTGGGTTCCTCTGCAGACA
TTCACCCAGCATCAAGGGGCTGTCAAGGCCGTAGCATGGTGTCCCTGGCA
GTCCAATGTCCTGGCAACAGGAGGGGGCACCAGTGATCGACACATTCGCA
TCTGGAATGTGTGCTCTGGGGCCTGTCTGAGTGCCGTGGATGCCCATTCC
CAGGTGTGCTCCATCCTCTGGTCTCCCCATTACAAGGAGCTCATCTCAGG
CCATGGCTTTGCACAGAACCAGCTAGTTATTTGGAAGTACCCAACCATGG
CCAAGGTGGCTGAACTCAAAGGTCACACATCCCGGGTCCTGAGTCTGACC
ATGAGCCCAGATGGGGCCACAGTGGCATCCGCAGCAGCAGATGAGACCCT
GAGGCTATGGCGCTGTTTTGAGTTGGACCCTGCGCGGCGGCGGGAGCGGG
AGAAGGCCAGTGCAGCCAAAAGCAGCCTCATCCACCAAGGCATCCGCTGA

What is claimed is:

1. A method of screening for a candidate anti-cancer agent, comprising
a. providing a cell expressing a Cdc20 variant and resistant to an anti-mitotic drug;
b. contacting the cell with an anti-mitotic drug and a test agent;
c. determining if the test agent reduces mitotic slippage as compared to a control; and
d. identifying the test agent as a candidate anti-cancer agent if the test agent reduces mitotic slippage as compared to the control.

2. The method of claim 1, wherein the cell is a cancer cell.

3. The method of claim 1, wherein the Cdc20 variant comprises an N-terminal truncation.

4-9. (canceled)

10. A method of inhibiting a cancer cell expressing a Cdc20 variant and resistant to an anti-mitotic drug comprising contacting the cancer cell with an agent that reduces the expression or activity of the Cdc20 variant and the anti-mitotic drug.

11. The method of claim 10, wherein the agent inhibits the binding of the Cdc20 variant with APC/C.

12. The method of claim 10, wherein the agent inhibits the expression of the Cdc20 variant.

13. The method of claim 10, wherein the agent increases the expression or activity of Cdc20 wild-type or a Cdc20 variant not resistant to the anti-mitotic drug.

14. The method of claim 13, wherein the agent is the Cdc20 wild-type or the Cdc20 variant not resistant to the anti-mitotic drug, or a nucleotide sequence coding for the same.

15. The method of claim 10, wherein the agent comprises residues 1-42 of SEQ ID NO: 2, or a functional fragment thereof.

16. A method of inhibiting a cancer cell expressing a Cdc20 variant and resistant to an anti-mitotic drug comprising contacting the cancer cell with an endonuclease and modifying the genome of the cancer cell, wherein the modification reduces or eliminates the expression of a Cdc20 variant resistant to an anti-mitotic drug or wherein the modification increases the expression of wild-type Cdc20.

17. The method of claim 16, wherein the endonuclease is a Cas9 nuclease and wherein the cancer cell is further contacted with one or more gRNA.

18. The method of claim 16, wherein the modification eliminates one or more Cdc20 alternate translation start sites.

19. The method of claim 18, wherein the alternate translation start site is located at positions 127-129 or positions 262-264 of SEQ ID NO: 1.

20. The method of claim 16, wherein the modification increases translation from the wild-type translation start site or increases translation of full length wild-type protein.

21. The method of claim 20, wherein the modification introduces a substitution in the wild-type translation start sites or modifies a promoter binding site.

22. The method of claim 16, wherein the modified cell is contacted with the anti-mitotic drug.

23-26. (canceled)


SEQUENCE LISTING (68 sequences)

SEQ ID NO: 1 - DNA, 1500 bp, Homo sapiens
  source 1..1500: mol_type = genomic DNA
atggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctga

SEQ ID NO: 2 - protein, 499 aa, Homo sapiens
MAQFAFESDLHSLLQLDAPIPNAPPARWQRKAKEAAGPAPSPMRAANRSHSAGRTPGRTPGKSSSKVQTTPSKPGGDRYIPHRSAAQMEVASFLLSKENQPENSQTPTKKEHQKAWALNLNGFDVEEAKILRLSGKPQNAPEGYQNRLKVLYSQKATPGSSRKTCRYIPSLPDRILDAPEIRNDYYLNLVDWSSGNVLAVALDNSVYLWSASSGDILQLLQMEQPGEYISSVAWIKEGNYLAVGTSSAEVQLWDVQQQKRLRNMTSHSARVGSLSWNSYILSSGSRSGHIHHHDVRVAEHHVATLSGHSQEVCGLRWAPDGRHLASGGNDNLVNVWPSAPGEGGWVPLQTFTQHQGAVKAVAWCPWQSNVLATGGGTSDRHIRIWNVCSGACLSAVDAHSQVCSILWSPHYKELISGHGFAQNQLVIWKYPTMAKVAELKGHTSRVLSLTMSPDGATVASAAADETLRLWRCFELDPARRREREKASAAKSSLIHQGIR

SEQ ID NO: 3 - DNA, 1374 bp, synthetic construct
  source 1..1374: mol_type = other DNA
atgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctga

SEQ ID NO: 4 - protein, 457 aa, synthetic construct [sequence omitted]

SEQ ID NO: 5 - DNA, 1239 bp, synthetic construct
  source 1..1239: mol_type = other DNA
atggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctga

SEQ ID NO: 6 - protein, 412 aa, synthetic construct [sequence omitted]

SEQ ID NO: 7 - DNA, 1497 bp, synthetic construct
  source 1..1497: mol_type = other DNA
gcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctga

SEQ ID NO: 8 - DNA, 1553 bp, synthetic construct
  source 1..1553: mol_type = other DNA
atggcacagttcgcgttcgagagtgacctgcactcgctgcttcacagggtttctccatgttggtcaggctgatcttgaactcccaacctcaggtgaagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctga

SEQ ID NO: 9 - DNA, 1605 bp, synthetic construct
  source 1..1605: mol_type = other DNA
atggcacagttcgcgttcgagagtgacctgcactcgctgcttcgattgggaagagaatagcaggcatgctggggagcggccgcaggaacccctagtgatggagttggccactccctctctgcgcgctcgctcgctcactgaggccgcccgctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctga

SEQ ID NO: 10 - RNA, 20 nt, synthetic construct
  source 1..20: mol_type = other RNA
ggggccactagggacaggat

SEQ ID NO: 11 - protein, 95 aa, Homo sapiens
MAQFAFESDLHSLLQLDAPIPNAPPARWQRKAKEAAGPAPSPMRAANRSHSAGRTPGRTPGKSSSKVQTTPSKPGGDRYIPHRSAAQMEVASFLL

SEQ ID NO: 12 - protein, 95 aa, unidentified
  source 1..95: note = Rhesus
MAQFAFESDLHSLLQLDAPIPNAPPARWQRKAKEASGPAPSPMRAANRSHSAGRTPGRTPGKSSSKVQTTPSKPGGDRYIPHRSAAQMEVASFLL

SEQ ID NO: 13 - protein, 95 aa, Mus musculus
MAQFVFESDLHSLLQLDAPIPNAPVARWQRKAKEATGPAPSPMRAANRSHSAGRTPGRTPGKSSSKVQTTPSKPGGDRFIPQRSASQMEVASFLL

SEQ ID NO: 14 - protein, 95 aa, unidentified
  source 1..95: note = Kangroo Rat
MAQFVFESDLHSLLQLDAPIPNAPLARWQRKAKEAAGPAPSPMQAANRSHSAGRTPGRTPGKSNSKTQTTPSKPGGDRYIPHRSASQMEVASFLL

SEQ ID NO: 15 - protein, 95 aa, Canis lupus
MAQFVFESDLHSLLQLDAPIPNAPPARWQRKAREAAGPAPSPMRAANRSHSAGRTPGRTPGKSSSKAQTTPSKPGGDRYIPHRSASQMEVASFLL

SEQ ID NO: 16 - protein, 103 aa, Gallus gallus
MAHFVFEADLHGLLKLDTPIPNAPPARWQRKAKESACPGPGPSPAASMSPMKPANRSYSGSKTPSKTPGKSGSKVQSTPTKAGGDRYIPNRSTMQMEMANFLL

SEQ ID NO: 17 - protein, 104 aa, Xenopus sp.
MAQFAFETDINSILKLDTPITNAPLARWQRKAKEGNCSSLNTSANTSTMSPMKTSNRSHSSSKTPSKTPGKSGPKMQGTPSRAGGDRFIPNRSAMQMDVASFLL

SEQ ID NO: 18 - protein, 26 aa, synthetic construct
AQFAFESDLHSLLQLDAPIPNAPPAR

SEQ ID NO: 19 - protein, 29 aa, synthetic construct
AQFAFESDLHSLLQLDAPIPNAPPARWQR

SEQ ID NO: 20 - protein, 30 aa, synthetic construct
AQFAFESDLHSLLQLDAPIPNAPPARWQRK

SEQ ID NO: 21 - protein, 10 aa, synthetic construct
MEVASFLLSK

SEQ ID NO: 22 - protein, 23 aa, synthetic construct
MEVASFLLSKENQPENSQTPTKK

SEQ ID NO: 23 - protein, 20 aa, synthetic construct
MRAANRSHSAGRTPGRTPGK

SEQ ID NO: 24 - protein, 10 aa, synthetic construct
MEVASFLLSK

SEQ ID NO: 25 - protein, 22 aa, synthetic construct
MEVASFLLSKENQPENSQTPTK

SEQ ID NO: 26 - protein, 23 aa, synthetic construct
MEVASFLLSKENQPENSQTPTKK

SEQ ID NO: 27 - RNA, 10 nt, synthetic construct
  source 1..10: mol_type = other RNA
gccaccatgg

SEQ ID NO: 28 - RNA, 10 nt, synthetic construct
  source 1..10: mol_type = other RNA
gctcttatgc

SEQ ID NO: 29 - RNA, 20 nt, synthetic construct
  source 1..20: mol_type = other RNA
gacgcacccattcctaacgc

SEQ ID NO: 30 - skipped

SEQ ID NO: 31 - RNA, 314 nt, synthetic construct
  source 1..314: mol_type = other RNA
gcgctcccatggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaa

SEQ ID NO: 32 - protein, 78 aa, Homo sapiens
MHPSPMHPLRAGSAKPRKPQARPPHPCGPPTDPTAPAGLRAELLANPVPRFRPLLANLAVTAISPIAVLPRWRWPASS

SEQ ID NO: 33 - protein, 73 aa, Mus musculus
MPRLRAGSAKQRKPQAQPPRPCGPPTDHTAPGGPRAELLANLVPRFRPPLANLEVTALSPNAVLLKWRWPASS

SEQ ID NO: 34 - protein, 78 aa, Sus sp.
MRPSPMHLPRAGSAKRRKPRGRPPRPCGPPTDPTAPAGPPAERRANPTPRCRPLPASLAAIAISPTAVPPRWRWPASS

SEQ ID NO: 35 - protein, 73 aa, Camelus sp.
MHPQRAGSAKPRKPRGPPPRPCGPPTDLIALAGPRAELPGNPAPRFRPLPANLAVTAISPIAVLPRWRWPASS

SEQ ID NO: 36 - protein, 73 aa, Bos taurus
MHPLRAGSAKRRKPRGRPPRLCGQPTDPTAPAGPQAELPANPAPSFRPLPANLAVTAISPIAMLPRWRWLASS

SEQ ID NO: 37 - protein, 73 aa, unidentified
  source 1..73: note = Kangroo Rat
MHLLRAGSARPRKRRGRPPHPCRPPTDPTAPAGPRAALLANLTPRLRPPLANLAVTAISPIVVLPRWRWPASS

SEQ ID NO: 38 - RNA, 141 nt, synthetic construct
  source 1..141: mol_type = other RNA
atggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaac

SEQ ID NO: 39 - RNA, 34 nt, synthetic construct
  source 1..34: mol_type = other RNA
tcccatggcacagttcgcgttcgagagtgacctg

SEQ ID NO: 40 - RNA, 30 nt, synthetic construct
  source 1..30: mol_type = other RNA
tccgcacagttcgcgttcgagagtgacctg

SEQ ID NO: 41 - DNA, 84 bp, synthetic construct
  source 1..84: mol_type = other DNA
atggcacagttcgcgttcgagagtgacctgcactcgctgcttcagctggatgcacccatccccaatgcaccccctgcgcgctgg

SEQ ID NO: 42 - DNA, 137 bp, synthetic construct
  source 1..137: mol_type = other DNA
atggcacagttcgcgttcgagagtgacctgcactcgctgcttcacagggtttctccatgttggtcaggctgatcttgaactcccaacctcaggtgaagctggatgcacccatccccaatgcaccccctgcgcgctgg

SEQ ID NO: 43 - DNA, 189 bp, synthetic construct
  source 1..189: mol_type = other DNA
atggcacagttcgcgttcgagagtgacctgcactcgctgcttcgattgggaagagaatagcaggcatgctggggagcggccgcaggaacccctagtgatggagttggccactccctctctgcgcgctcgctcgctcactgaggccgcccgctggatgcacccatccccaatgcaccccctgcgcgctgg

SEQ ID NO: 44 - RNA, 228 nt, synthetic construct
  source 1..228: mol_type = other RNA
atggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggt

SEQ ID NO: 45 - RNA, 118 nt, synthetic construct
  source 1..118: mol_type = other RNA
atggcacagttcgcgttcgagagtgacctgcactcgctgcttcagctggatgcacccatccccaatgcaccccctgcgcgctggcagcgccgcaggcccggccccctcacccatgcgg

SEQ ID NO: 46 - RNA, 126 nt, synthetic construct
  source 1..126: mol_type = other RNA
atggcacagttcgcgttcgagagtgacctgcatcgctgcttcagctggatgcacccatccccaatgcaccccctgcgcgctggcagcgcaaaggaagccgcaggcccggccccctcacccatgcgg

SEQ ID NO: 47 - RNA, 127 nt, synthetic construct
  source 1..127: mol_type = other RNA
atggcacagttcgcgttcgagagtgacctgcactcgctgcttcagctggatgcacccatccccaatgcaccccctgcgcgctggcagcgcaaaggaagccgcaggcccggccccctcacccatgcgg

SEQ ID NO: 48 - RNA, 133 nt, synthetic construct
  source 1..133: mol_type = other RNA
atggcacagttcgcgttcgagagtgacctgcactcgctgcttcagctggatgcacccatccccaatgcaccccctgcgcgctggcagcgcaaaggccaaggaagccgcaggcccggccccctcacccatgcgg

SEQ ID NO: 49 - RNA, 229 nt, synthetic construct
  source 1..229: mol_type = other RNA
atggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacaagcgccggcaggactccgggccgaactcctggcaaatccagttccaaggttcagaccactcctagcaaacctggcggt

SEQ ID NO: 50 - RNA, 229 nt, synthetic construct; identical to SEQ ID NO: 49
  source 1..229: mol_type = other RNA

SEQ ID NO: 51 - RNA, 229 nt, synthetic construct
  source 1..229: mol_type = other RNA
atggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacaggcgccggcaggactccgggccgaactcctggcaaatccagttccaaggttcagaccactcctagcaaacctggcggt

SEQ ID NO: 52 - protein, 7 aa, synthetic construct
DAPIPNA

SEQ ID NO: 53 - protein, 102 aa, synthetic construct
MAQFAFESDLHSLLQLDAPIPNAPPARWQRKAKEAAGPAPSPMRAANRSHSAGRTPGRTPGKSSSKVQTTPSKPGGDRYIPHRSAAQMEVASFLLSKENQPE

SEQ ID NO: 54 - protein, 47 aa, synthetic construct
MAQFAFESDLHSLLQLDAPIPNAPPARWQRKAKEAAGPAPSPMRAAN

SEQ ID NO: 55 - protein, 10 aa, synthetic construct
MAQFAFESDL

SEQ ID NO: 56 - protein, 28 aa, synthetic construct
MAQFAFESDLHSLLQLDAPIPNAPPARW

SEQ ID NO: 57 - protein, 23 aa, synthetic construct
MAQFAFESDLHSLLHRVSPCWSG

SEQ ID NO: 58 - protein, 19 aa, synthetic construct
MAQFAFESDLHSLLRLGRE

SEQ ID NO: 59 - protein, 76 aa, synthetic construct
MAQFAFESDLHSLLQLDAPIPNAPPARWQRKAKEAAGPAPSPMRAANRSHSAGRTPGRTPGKSSSKVQTTPSKPGG

SEQ ID NO: 60 - protein, 59 aa, synthetic construct
MHPSPMHPLRAGSAKPRKPQARPPHPCGPPTDPTAPAGLRAELLANPVPRFRPLLANLA

SEQ ID NO: 61 - protein, 23 aa, synthetic construct
MHPSPMHPLRAGSAAGPAPSPMR

SEQ ID NO: 62 - protein, 26 aa, synthetic construct
MHPSPMHPLRAGSAKEAAGPAPSPMR

SEQ ID NO: 63 - protein, 28 aa, synthetic construct
MHPSPMHPLRAGSAKAKEAAGPAPSPMR

SEQ ID NO: 64 - protein, 60 aa, synthetic construct
MHPSPMHPLRAGSAKPRKPQARPPHPCGPPTDPTSAGRTPGRTPGKSSSKVQTTPSKPGG

SEQ ID NO: 65 - protein, 60 aa, synthetic construct
MHPSPMHPLRAGSAKPRKPQARPPHPCGPPTDPTGAGRTPGRTPGKSSSKVQTTPSKPGG

SEQ ID NO: 66 - protein, 5 aa, synthetic construct
MAQFA

SEQ ID NO: 67 - protein, 18 aa, synthetic construct
CCCATGGCACAGTTCGCG

SEQ ID NO: 68 - protein, 18 aa, synthetic construct
CCCATGGCATAGTTCGCG